(19) United States
(12) Patent Application Publication
(10) Pub. No.: US 2025/0223612 A1
(43) Pub. Date: Jul. 10, 2025

(54) ARTIFICIAL EXPRESSION CONSTRUCTS FOR SELECTIVELY MODULATING GENE EXPRESSION IN INTERNEURONS

(71) Applicants: Allen Institute, Seattle, WA (US); Seattle Children's Hospital d/b/a Seattle Children's Research Institute, Seattle, WA (US)

(72) Inventors: Jonathan Ting, Lake Forest Park, WA (US); Boaz P. Levi, Seattle, WA (US); John K. Mich, Seattle, WA (US); Edward Sebastian Lein, Mercer Island, WA (US); Franck Kalume, Bothell, WA (US)

(73) Assignees: Allen Institute, Seattle, WA (US); Seattle Children's Hospital d/b/a Seattle Children's Research Institute, Seattle, WA (US)

(21) Appl. No.: 19/007,152

(22) Filed: Dec. 31, 2024

Related U.S. Application Data
(63) Continuation of application No. 17/283,232, filed on Apr. 6, 2021.
(60) Provisional application No. 62/810,281, filed on Feb. 25, 2019.
(60) Provisional application No. 62/749,012, filed on Oct. 22, 2018.
(60) Provisional application No. 62/742,835, filed on Oct. 8, 2018.

(51) Int. Cl. C12N 15/86; A01K 67/0278; A61K 35/30; C12N 5/0793

(52) U.S. Cl. CPC C12N 15/86; A01K 67/0278; A61K 35/30; C12N 5/0619; A01K 2217/052; A01K 2227/105; A01K 2227/106; C12N 2510/00; C12N 2750/14043

(57) ABSTRACT

Artificial expression constructs for selectively modulating gene expression in selected central nervous system cell types are described. The artificial expression constructs can be used to selectively express synthetic genes or modify gene expression in GABAergic interneurons.

### CROSS-REFERENCE TO RELATED APPLICATIONS

This application is a continuation of U.S. patent application Ser. No. 17/283,232 filed on Apr. 6, 2021, which is a U.S. National Phase based on International Patent Application No. PCT/US2019/054539 filed on Oct. 3, 2019, which claims priority to U.S. Provisional Patent Application Nos. 62/742,835 filed Oct. 8, 2018; 62/749,012 filed Oct. 22, 2018; and 62/810,281 filed Feb. 25, 2019, each of which is incorporated herein by reference in its entirety as if fully set forth herein.

### STATEMENT REGARDING FEDERALLY SPONSORED RESEARCH OR DEVELOPMENT

This invention was made with government support under grant RF1MH114126 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the XML file containing the Sequence Listing is 3CR7287.XML. The text file is 210,276 bytes, was created on Dec. 23, 2024, and is being submitted electronically via Patent Center.

### FIELD OF THE DISCLOSURE

The current disclosure provides artificial expression constructs for selectively modulating gene expression in selected central nervous system cell types. The artificial expression constructs can be used to selectively express synthetic genes or modify gene expression in GABAergic forebrain interneurons.

### BACKGROUND OF THE DISCLOSURE

GABAergic interneurons play critical roles in central nervous system processing as well as development. Dysfunction of these cells can also contribute to numerous neuropsychiatric disorders, such as schizophrenia and autism. GABAergic interneurons also play a role in epilepsy.

Cell-type or cell-class specific gene delivery using non-pathogenic recombinant adeno-associated virus (rAAV) is showing increasing promise for the treatment of diverse diseases. Inclusion within rAAVs of one or more cis-acting DNA-control elements, such as specific promoters or enhancers, has been beneficial to provide specificity of expression within particular target cells, including specific cell types or cell classes in the brain.

Dimidschstein and colleagues (Nat Neurosci 19(12):1743-1749, 2016) developed a rAAV that permits largely selective gene expression in GABAergic interneurons within the telencephalon. This rAAV includes a 527 bp enhancer sequence (referred to as mI56i or mDIx) from the intergenic interval between the distal-less homeobox 5 and 6 genes (DIx5/6), which are naturally expressed by forebrain GABAergic interneurons during embryonic development. The construct of Dimidschstein et al. is available on Addgene as ID #83900 (in which the enhancer drives eGFP expression). Additional constructs which employ the murine or human I56i enhancer to drive various transgenes are available through Addgene, such as Plasmid ID #s 83899 (driving GCaMP6f expression), 83898 (driving ChR2 expression), 83895 (driving synthetic eGFP expression), 89897 (driving hM3DREADD expression), 83896 (driving hM4Di expression), and 83894 (driving synthetic tdTomato expression). See also U.S. Patent Publication No. US2018/0078658.

Additionally, the mI56i enhancer has previously been used to reliably target reporter genes in a pattern very similar to the normal patterns of DIx5/6 expression during embryonic development (Zerucha et al., J Neuroscience 20:709-721, 2000; Stühmer et al., Cerebral Cortex 12:75-85, 2002; Stenman et al., J Neuroscience 23:167-174, 2003; Monory et al., Neuron. 51:455-455, 2006; Miyoshi et al., J Neuroscience 30:1532-1594, 2010).

One significant drawback to using rAAVs as a gene-delivery system is the restricted packaging limit of AAVs; this is particularly limiting to the inclusion of lengthy genetic control and expression elements. In addition, many existing interneuron-specific rAAV expression constructs can provide weak gene expression reducing their usefulness in research and therapeutic uses.

### SUMMARY OF THE DISCLOSURE

The current disclosure overcomes drawbacks of the prior art by providing engineered enhancer elements that provide rapid and strong cell-specific expression of heterologous encoding sequences in forebrain GABAergic interneurons.

In particular embodiments, the artificial enhancer elements include a concatemerized core of a I56i enhancer. These artificial enhancer elements provide more rapid onset of transgene expression compared to a single full length original (native) enhancer.

In particular embodiments, the I56i enhancer core can be derived from, for example the human, murine, or zebrafish I56i enhancer (SEQ ID NOs. 1, 4, and 5 respectively). The selected cores of the I56i enhancer can include SEQ ID NO: 2 (core shared by human and mouse) or SEQ ID NO: 6 (zebrafish core). In particular embodiments, the cores are concatemerized. For example, SEQ ID NO: 3 provides a three-copy concatemer of the selected human/murine I56i core while SEQ ID NO: 7 provides a three-copy concatemer of the selected zebrafish I56i core.

Of particular interest, the synthetic 3× human/murine core (referred to herein as the 3×hI56iCore; SEQ ID NO: 3) is shorter than the original full length enhancer sequence reported in Dimidschstein et al. (Nat Neurosci 19(12):1743-1749, 2016), despite being a 3× concatemer. Thus, this concatemerized core provides more room for cargo genes linked to the enhancer, which is highly desirable. Moreover, the peak level of transgene expression driven by the 3×hI56iCore enhancer is much greater than simply three times the level of the original single full-length original enhancer.

The engineered concatemerized I56i cores disclosed herein enable new and improved gene delivery vectors that are particularly useful for achieving selective transgene expression in forebrain GABAergic interneurons in diverse animal species, including humans.

### BRIEF DESCRIPTION OF THE FIGURES

Many of the drawings submitted herein are better understood in color. Applicants consider the color versions of the drawings as part of the original submission and reserve the right to present color images of the drawings in later proceedings.

FIG. 1: Virus CN1244/PHP.eB. 10¹¹ genome copies delivered intravenously (IV) in adult mouse. PHP.eB encodes for a capsid originating from AAV9 that allows efficient AAV transit across the mouse blood brain barrier, which enables delivery of AAV vectors in a brain-wide fashion. This capsid differs from AAV9 such that amino acids starting at residue 586: SAQA (SEQ ID NO: 98) are changed to SDGTLAVPFKA (SEQ ID NO: 33). The Gad2-T2A-nls-mCherry reporter marks nearly all inhibitory neurons in the mouse brain (here shown V1 visual cortex), and the delivered CN1244/PHP.eB virus drives specific SYFP2 reporter activity in forebrain GABAergic neurons.

FIGS. 2A,2B: Comparison of CN1244 vs CN1389 vs CN1390. (FIG. 2A) Schematic representations of three vector constructs, CN1390, CN1389, and CN1244 (CN1203 scAAV). Key: hI56i—full length human enhancer (black box; SEQ ID NO: 1); selected hI56i core (grey box; SEQ ID NO: 2) and 3× concatemer of core (grey boxes; SEQ ID NO: 3); minBG—minimal beta globin promoter; SYFP2—super yellow fluorescent protein 2; WPRE3—woodchuck hepatitis virus post-transcriptional regulatory element 3; BGHpA—bovine growth hormone polyA sequence; L-ITR and R-ITR-—Adeno-associated virus-2 (AAV2) inverted terminal repeats (ITRs). (FIG. 2B) Fluorograph images showing relative expression of SYFP2 from AAV vector constructs CN1244, CN1389, and CN1390. Adult wild type mice were retro-orbitally injected with 1E+11 genome copies of the indicated viruses. Animals were maintained for 3-4 weeks, then euthanized, with brains extracted and sliced, followed by live tissue epifluorescence imaging of native fluorescence. Exposure times were matched to allow direct comparison of transgene expression levels. The first three panels are a 500 msec exposure for each of the indicated constructs; the fourth panel is a shorter (50 msec) exposure image of CN1390. CN1390 with the engineered concatemerized core demonstrated strong and more rapid transgene expression.

FIG. 3: CN1390 retains cell type specificity for the pan-GABAergic neuron population. Cortical/hippocampal brain slice cultures were prepared from P5-10 Gad2-IRES-Cre het/Ai75 het animals. One hour after culturing, CN1390 viral suspension was pipetted onto the slice surface to transduce brain cell types. At 10 DIV/10 DPI, native fluorescence was imaged in green and red channels on a Nikon inverted microscope. 10 DIV/10 DPI. DIV: days in vitro, DPI: days post infection.

FIGS. 4A, 4B: Comparison of CN1244 vs CN1390 in non-human primate ex vivo brain slice culture. (FIG. 4A) Fluorograph images showing relative expression of SYFP2 from AAV vector constructs CN1244 and CN1390. Neocortical slices were cultured from adult macaque brain and infected with nominally-matched titers of the indicated viruses. Brain slice cultures were maintained in the incubator for 4 days in vitro, 4 days post infection (4 DIV/4 DPI), then used for live tissue epifluorescence imaging of native fluorescence. Exposure times were matched to allow direct comparison of transgene expression levels. CN1390 with the engineered concatemerized core demonstrated strong and more rapid transgene expression. (FIG. 4B) Fluorograph images showing relative expression of SYFP2 from AAV vector constructs CN1244 and CN1390. Hippocampal slices were cultured from adult macaque brain and infected with nominally-matched titers of the indicated viruses. Brain slice cultures were maintained in the incubator for 6 days in vitro, 6 days post infection (6 DIV/6 DPI), then used for live tissue epifluorescence imaging of native fluorescence. Exposure times were matched to allow direct comparison of transgene expression levels. CN1390 with the engineered concatemerized core demonstrated strong and more rapid transgene expression.

FIGS. 5A-5E: CN1390 exhibits rapid onset of transgene expression in human ex vivo brain slices. Human ex vivo neocortical brain slice cultures were prepared from live neurosurgical specimens as described in Ting et al., Scientific Reports 8(1):8407, 2018. One hour after culturing, CN1390 viral suspension was pipetted onto the slice surface to transduce brain cell types. At 1, 3, and 6 DIV/DPI, native SYFP2 fluorescence was imaged using matched exposure times on a Nikon microscope. FIGS. 5A-5D illustrate rapid viral-genetic labeling of human neocortical interneurons for targeted patch clamp recording and analysis. (FIG. 5A) Time course of virus-mediated YFP expression following human brain slice transduction with CN1390 eB. (FIG. 5B) Expanded view of the boxed region in (FIG. 5A). (FIG. 5C) High magnification view of a virus labeled interneuron with bipolar morphology. (FIG. 5D) Example whole cell recordings from four different virus-labeled YFP+ human interneurons demonstrating diverse firing patterns to supra-threshold current injection. (FIG. 5E) At various times in culture, slices were taken for terminal patch clamp recording analysis to establish the firing properties of labeled neurons. Functional analysis of human neocortical interneuron firing patterns and electrical properties by patch clamp recording was feasible as early as 40 hours post-infection with CN1390 AAV-PHP.eB virus.

FIG. 6: CN1390 maintains GABAergic cell class selectivity. At 7 to 34 DIV/DPI, virally transduced human organotypic slices from 4 unique human donors were transduced, dissociated, and 234 single SYFP2+ cells were FACS sorted from glia and debris-depleted cell suspensions, and profiled by single cell RNA-seq (SMARTer V.4). These cells were mapped to the existing MTG cell type taxonomy. Bars at the bottom of the taxonomy indicate the number of SYFP+ cells that mapped to the final leaf. Circles farther up the taxonomy indicate the number of cells that could only be mapped to that branch point. Note that cells from all major GABAergic classes were labeled, and no glutamatergic or glial cells were recovered. The listed cell types from top to bottom are: GABAergic types; 3 Inh L1-2 PAX-6 CDH12, 4 Inh L1-2 PAX6 TNF AIP8L3, 5 Inh L1 SST NMBR (ADARB2+), 6 Inh L1-4 LAMP5 LCP2 (rosehip), 7 Inh L1-2 LAMP5 DBP, 8 Inh L2-6 LAMP5 CA1 (lgtp), Inh L1 SST CHRNA4 (ADARB2+), 14 Inh L1-2 GAD1 MC4R (ADARB2+), 15 Inh L1-2 SST BAGE2 (ADARB2+), 17 Inh L1-3 PAX6 SYT6 (Sncg), 19 Inh L1-2 VIP TSPAN12, 20 Inh L1-4 VIP CHRNA6, 21 Inh L1-3 VIP ADAMTSL1, 22 Inh L1-4 VIP PENK, 27 Inh L2-6 VIP QPCT, 28 Inh L3-6 VIP HS3ST3A1, 29 Inh L1-2 VIP PCDH20, 31 Inh L2-5 VIP SERPINF1, 32Inh L2-5 VIP TYR, 37 Inh L1-3 VIP CHRM2, 38 Inh L2-4 VIP CBLN1, 39 Inh L1-3 VIP CCDC184, 40 Inh L1-3 VIP GGH, 42 Inh L1-2 VIP LBH, 43 Inh L2-3 VIP CASC6, 45 Inh L2-4 VIP SPAG17, 46 Inh L1-4 VIP OPRM1, Inh L3-6 SST NPY (Chodl), 52 Inh L3-6 SST HPGD, 55 Inh L4-6 SST B3GAT2, 56 Inh L5-6 SST KLHDC8A, 57 Inh L5-6 SST NPM1P10, 58 Inh L4-6 SST GXYLT2, 59 Inh L4-5 SST STK32A, 62 Inh L1-3 SST CALB1, 63 Inh L3-5 SST ADGRG6, 64 Inh L2-4 SST FRZB, 65 Inh L5-6 SST TH, 66 Inh L5-6 GAD1 GLP1R (LHX6+), 68 Inh L5-6 PVALB LGR5, 71 Inh L4-5 PVALB MEPE, 73 Inh L2-4 PVALB WFDC2, 74 Inh L4-6 PVALB SULF1, 75 Inh L5-6SST MIR548F2, 76 Inh L2-5 PVALB SCUBE3 (chandelier), Excitatory types; 82 Exc L2-5 LAMP5LTK, 83 Exc L2-4 LINC00507 GLP2R, 84 Exc L2-3 LINC00507 FREM3, 85 Exc L5-6 THEMIS C1QL3, 87 Exc L3-4 RORB CARM1P1, 89 Exc L3-5 RORB ESR1, 90 Exc L3-5 RORB COL22A1, 92 Exc L3-5 RORB FILIP1L, 93 Exc L3-5 RORB TWIST2, 96 Exc L4-5 RORB FOLH1B, 98 Exc L4-6 RORB SEMA3E, 99 Exc L4-5 RORB DAPK2, 100 Exc L5-6 RORB TTC12, 101 Exc L4-6 RORB C1R, Exc L4-5 FEZF2 SCN4B (PT), 102 Exc L5-6 THEMIS DCSTAMP, 103 Exc L5-6 THEMIS CRABP1, 104 Exc L5-6 THEMIS FGF10, 105 Exc L4-6 FEZF2 IL26 (NP), 106 Exc L5-6 FEZF2 ABO, 107 Exc L6 FEZF2 SCUBE1, 108 Exc L5-6 SLC17A7 IL15, 109 Exc L6 FEZF2 OR2T8, 110 Exc L5-6 FEZF2 EFTUD1P1, Glial types; OPC L1-6 PDGFRA, Astro L1-6 FGFR3 SLC14A1, Astro L1-2 FGFR3 GFAP, Oligo L1-6 OPALIN, Endo L2-6 NOSTRIN, AND Micro L1-3 TYROBP.

FIG. 7: Fast expression from CN1390 allows assessment of human circuit connectivity. Human neocortical organotypic slice was transduced with CN1390 and AAV-hSyn1-dTomato for 2.5 days. After only two and half days in culture, GABAergic and all neuronal cells can be labeled in culture using CN1390 and AAV-hSyn1-dTomato, respectively. Human synapsin 1 (hSyn1) is a well-known pan-neuronal promoter. This allows the assessment of connectivity between prospectively virally marked patched cells (labeled by cascade blue). The fluorescent dyes listed in the bottom left corner of the fluorescent image are (from top to bottom): panGABA-SYFP, hSyn1-tdTomato, and Fill-Blue.

FIGS. 8A, 8B: All major classes of human neocortical GABAergic neurons are marked by CN1390. (FIG. 8A) Multiplexed FISH using HCR v3.0 reveals major classes of GABAergic neurons labeled by somatostatin (SST), parvalbumin (PVALB), or vasoactive intestinal peptide (VIP) genes. Labeling by CN1390 in 350 um thick neocortical brain slice culture is shown. Text on the left image of FIG. 8A are as follows: (top left) Pial surface; (top right) Lipofuscin, PVALB, SST, VIP, and SYFP; and (bottom left) Hu, 350 μm Slice, Virus CN1390eB, 7 DIV/DPI. (FIG. 8B) Prospective cell class marking for physiology, connectivity and morphology. Multiplexed FISH reveals molecular identities of the CN1390-labeled cell classes and some of the patched cells that were back-filled with neurobiotin and visualized by Streptavidin-BV421. The left image of FIG. 8B is labeled with SYFP, SST, VIP, PVALB, and Lipofuscin. The right image of FIG. 8B is labeled Biocytin-BV421. All these cells show GABAergic cell morphology and most were marked by expression of SYFP2 from CN1390.

FIGS. 9A-9F. AAV vector reagents to reverse Dravet Syndrome (DS) symptoms in Scn1a^{+/-} mice. (9A) Vectors to deliver epitope-tagged Nav genes of bacterial origin (NavBacs). The Nav genes shown here are NavMs (from Magnetococcus marinus), NavBp (from Bacillus pseudofirmus), and NavSheP-D60N (from Shewanella putrifaciens with an engineered D60N mutation). These examples all have N-terminal epitope tags (hexahistidine in the case of CN1367, or 3×HA for CN1498, CN1499, and CN1500). hI56i refers to the full-length I56i enhancer of SEQ ID NO: 1; 3×hI56iCore refers to the concatemerized core of the I56i enhancer (SEQ ID NO: 3); (9B) Graded expression levels from NavBac vectors. (9C) Weak but detectable expression in Pvalb interneurons from vector CN1367. (9D) Trend towards seizure protection with vector CN1367. (9E) Vector 1500 drives high-level expression in Pvalb⁺ and Pvalb⁻ interneurons throughout cortex. (9F) Abundant production of HA-tagged NavBacs in cell bodies and proximal processes with vectors 1498 and 1500, but not 1499.

FIG. 10. CN1500 rAAV vector substantially reverses febrile seizures in Scn1a^{+/-} mice. Febrile seizure assay shown as internal temperature where a seizure is first detected. (Top) Circles show Scn1a^{+/-} mice untransduced with AAVs, while the diamonds represent animal that were transduced with CN1500. The large dot and error bars represent the average +/− SEM for each group of animals. (Bottom) Trends of the same data are shown as the percentage of mice in each group that remain seizure free at different temperatures using a Kaplan-Meier curve.

FIGS. 11A, 11B: Conservation of I56i enhancer sequences. (FIG. 11A) alignment of the human (SEQ ID NO: 1) I56i, murine (SEQ ID NO: 4) I56i, and zebrafish (SEQ ID NO: 5) I46i enhancer sequences. Residues shared by all three sequences are highlighted in light gray; those shared by the murine and human sequences are highlighted in dark gray. The core sequence (SEQ ID NO: 2) corresponds to positions 268-398 of the illustrated human sequence. The mouse and human I56i enhancer core sequences are exactly identical (100% sequence identity), as this is an ultraconserved enhancer sequence. It is also highly similar with the zebrafish genomic sequence, and the orthologous zebrafish enhancer (called I46i) has been used in many contexts over the years to drive transgene expression in neocortical interneurons, including for mouse neocortical interneurons. (FIG. 11B) graph illustrating the similarity between the human, murine, and zebrafish enhancer sequences. Graph shows (from right to left, and as labeled) Similarity, Absolute Complexity, and Absolute Complexity (human I56i).

FIG. 12: the sequence and indicated features of construct CN1389 pAAV-hI56i(core)-minBG-SYFP2-WPRE3-BGHpA (SEQ ID NO: 41). Select restriction endonuclease sites are indicated, as are the regions corresponding to different parts of the construct.

FIG. 13: the sequence and indicated features of construct CN1390 pAAV-3×hI56i(core)-minBG-SYFP2-WPRE3-BGHpA (SEQ ID NO: 42). Select restriction endonuclease sites are indicated, as are the regions corresponding to different parts of the construct.

FIG. 14: the sequence and indicated features of construct CN1203 scAAV-hI56i-minbGlobin-SYFP2-WPRE3-BGHpA (SEQ ID NO: 43). Select restriction endonuclease sites are indicated, as are the regions corresponding to different parts of the construct.

FIG. 15. Features of exemplary vectors disclosed herein.

FIG. 16. Artificial expression constructs within the teaching of the current disclosure. Each construct begins with a concatemerized core of the hI56i core (e.g., SEQ ID NO: 3 or 7) designated as *. The following abbreviations are also used: Beta-Globin minimal promoter (minB, referred to as minBglobin elsewhere herein), Minimal cytomegalovirus promoter (minC, referred to as minCMV elsewhere herein), Mutated minimal cytomegalovirus promoter (mut), Minimal rhodopsin promoter (minR, referred to as minRho elsewhere herein), Cytomegalovirus promoter (CMV), Simian vacuolating virus 40 promoter (SV40), Hsp68 minimal promoter (H68, referred to as proHSP68 elsewhere herein), Rous Sarcoma Virus long-terminal repeat promoter (RSV), Fluorescent protein (FP), Blue fluorescent protein (BFP), Cyan fluorescent protein (CFP), Green fluorescent protein (GFP), Orange fluorescent protein (OFP), Red fluorescent protein (RFP), Far red fluorescent protein (fRFP), Yellow fluorescent protein (YFP), Luciferase (Luc), Enzyme (enz), Transcription factor (TF), Receptor (rec), Cellular trafficking protein (CTP), Signaling molecule (SM), Neurotransmitter (NT), Calcium reporter (CR), hannel rhodopsin (ChR), Guide RNA (gRNA), Nuclease (Nuc), Woodchuck hepatitis virus post-transcriptional response element (W, referred to as WPRE3 elsewhere herein), Bovine growth hormone polyadenylation signal (bG, referred to as bGHpA elsewhere herein), Simian vacuolating virus 40 polyadenylation signal (S, referred to as SV40pA elsewhere herein), Internal ribosome entry site 2 (I2, referred to as IRES2 elsewhere herein), and 2A skipping elements (T2A, P2A, E2A, and F2A).

FIG. 17. Additional sequences supporting the disclosure: hI56i enhancer: (SEQ ID NO: 1); Core of the hI56i enhancer: (SEQ ID NO: 2); 3×hI56iCore, Triply Concatamerized Core of the hI56i enhancer: (SEQ ID NO: 3); Murine I56i Enhancer (core is the same as human): (SEQ ID NO: 4); Zebrafish I46i Enhancer: (SEQ ID NO: 5); Core of the Zebrafish I46i Enhancer: (SEQ ID NO: 6); 3× Concatamerized Core of the Zebrafish I46i Enhancer: (SEQ ID NO: 7); Beta-Globin Minimal Promoter pBGmin/minBGlobin/minBGprom): (SEQ ID NO: 8); minCMV Promoter: (SEQ ID NO: 9); Mutated minCMV Promoter (Sacl RE site removed): (SEQ ID NO: 10); minRho Promoter: (SEQ ID NO: 11); Hsp68 minimal Promoter (proHsp68): (SEQ ID NO: 12); SYFP2: (SEQ ID NO: 13); EGFP: (SEQ ID NO: 14); Optimized Flp recombinase (FIpO): (SEQ ID NO: 15); Improved Cre recombinase (iCre): (SEQ ID NO: 16); NavMs, endogenous sequence: (SEQ ID NO: 17); NavMs, codon optimized, with N-terminal 3× HA tag and linker: (SEQ ID NO: 18); NavMs, codon optimized, with N-terminal His tag and linker: (SEQ ID NO: 19); NavBp, endogenous sequence: (SEQ ID NO: 20); NavBp, codon optimized, with N-terminal 3× HA tag: (SEQ ID NO: 21); NavSheP-D60N, codon optimized, with N-terminal 3× HA tag: (SEQ ID NO: 22); NavSheP endogenous sequence: (SEQ ID NO: 23); WPRE3: (SEQ ID NO: 24); BGHpA: (SEQ ID NO: 25); P2A Encoding Sequence: (SEQ ID NO: 26); P2A: (SEQ ID NO: 27); T2A: (SEQ ID NO: 28); E2A: (SEQ ID NO: 29); F2A: (SEQ ID NO: 30); N-terminal 3×HA tag: (SEQ ID NO: 31); N-terminal 3× HA tag: (SEQ ID NO: 32); PHP.eB capsid: (SEQ ID NO: 90); AAV9 VP1 capsid protein: (SEQ ID NO: 34); tet-Transactivator version 2 (tTA2): (SEQ ID NO: 35); CN1367—The portion between L-ITR and R-ITR: positions 142-2984: (SEQ ID NO: 36); CN1500—The portion between L-ITR and R-ITR: positions 142-2976: (SEQ ID NO: 37); CN1498—The portion between L-ITR and R-ITR: positions 142-2943: (SEQ ID NO: 38); CN1499—The portion between L-ITR and R-ITR: positions 142-2946: (SEQ ID NO: 39); CN1244—The portion between L-ITR and R-ITR: positions 142-2042: (SEQ ID NO: 40); CN1389—The portion between L-ITR and R-ITR corresponds to positions 142-1660: (SEQ ID NO: 41); CN1390—The portion between L-ITR and R-ITR corresponds to positions 142-1897: (SEQ ID NO: 42); CN1203—The portion between L-ITR and R-ITR corresponds to positions 183-2052: (SEQ ID NO: 43); Lactase (SEQ ID NO: 44); Lipase (SEQ ID NO:45); Helicase (SEQ ID NO: 46); Amylase (SEQ ID NO: 47); α-glucosidase (SEQ ID NO: 48); Transcription factor SP1 (SEQ ID NO: 49); Transcription factor AP-1 (SEQ ID NO: 50); Heat shock factor protein 1 (SEQ ID NO: 51); CCAAT/enhancer-binding protein (C/EBP) β isoform a (SEQ ID NO: 52); Octamer-binding protein 1 (SEQ ID NO: 53); Transforming growth factor receptor β1 (SEQ ID NO: 54); Platelet-derived growth factor receptor (SEQ ID NO: 55); Epidermal growth factor receptor (SEQ ID NO: 56); Vascular endothelial growth factor receptor (SEQ ID NO: 57); Interleukin 8 receptor α (SEQ ID NO: 58); Caveolin (SEQ ID NO: 59); Dynamin (SEQ ID NO: 60); Clathrin heavy chain 1 isoform 1 (SEQ ID NO: 61); Clathrin heavy chain 2 isoform 1 (SEQ ID NO: 62); Clathrin light chain A isoform a (SEQ ID NO: 63); Clathrin light chain B isoform a (SEQ ID NO: 64); Ras-related protein Rab-4A isoform 1 (SEQ ID NO: 65); Ras-related protein Rab-11A (SEQ ID NO: 66); Platelet-derived growth factor (SEQ ID NO: 67); Transforming growth factor-β3 (SEQ ID NO: 68); Nerve growth factor (SEQ ID NO: 69); Epidermal growth factor (SEQ ID NO: 70); GTPase HRas (SEQ ID NO: 71); Cocaine And Amphetamine Regulated Transcript (Chain A) (SEQ ID NO: 72); Protachykinin-1 (SEQ ID NO: 73); Substance P (SEQ ID NO: 74); Oxytocin-neurophysin 1 (SEQ ID NO: 75); Oxytocin (SEQ ID NO: 76); Somatostatin (SEQ ID NO: 77); Myosin light chain kinase, Green fluorescent protein, Calmodulin chimera (Chain A) (SEQ ID NO: 78); Genetically-encoded green calcium indicator NTnC (chain A) (SEQ ID NO: 79); Calcium indicator TN-XXL (SEQ ID NO: 80); BRET-based auto-luminescent calcium indicator (SEQ ID NO: 81); Calcium indicator protein OeNL (Ca2+)-18u (SEQ ID NO: 82); GCaMP6m (SEQ ID NO: 99); GCaMP6s (SEQ ID NO: 100); GCaMP6f (SEQ ID NO: 101); Channelopsin 1 (SEQ ID NOs: 83 and 102); Channelrhodopsin-2 (SEQ ID NOs: 84 and 103); CRISPR-associated protein (Cas) (SEQ ID NO: 85); Cas9 (SEQ ID NO: 86); CRISPR-associated endonuclease Cpf1 (SEQ ID NO: 87); Ribonuclease 4 or Ribonuclease L (SEQ ID NO: 88); Deoxyribonuclease II β (SEQ ID NO: 89); Sodium channel protein type 1 subunit alpha (SEQ ID NO: 104); Potassium voltage-gated channel subfamily KQT member 2 (SEQ ID NO: 105); and Voltage-dependent L-type calcium channel subunit alpha-1C (SEQ ID NO: 106).

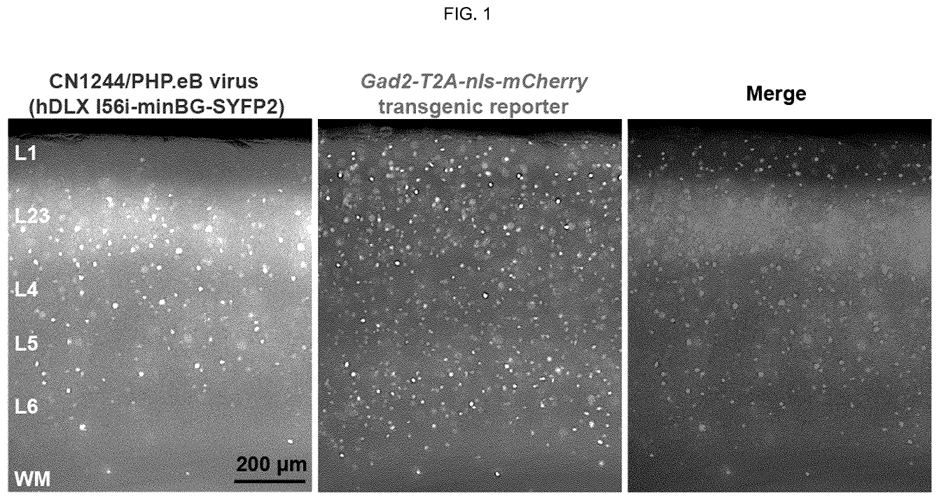

### DETAILED DESCRIPTION

To fully understand the biology of the brain, different cell types need to be distinguished and defined. To identify and/or study these different cell types, vectors that can selectively label and perturb them need to be identified. In mouse, recombinase driver lines have been used to great effect to label cell populations that share marker gene expression. However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans, and recent advances in single-cell profiling, such as single-cell RNA-seq (Tasic et al., Nature 563, 72-78 (2018); Tasic 2016, Nat Neurosci 19, 335-346) and surveys of neural electrophysiology and morphology (Gouwens 2019, Nat Neurosci 22, 1182-1195), have revealed that many recombinant driver lines label heterogeneous mixtures of cell types, and often include cells from multiple subclasses. For example, the Rbp4-Cre mouse driver line, which is commonly used to label layer 5 (L5) neurons, also labels cells with drastically different connectivity patterns: L5 intratelencephalic (IT, also called cortico-cortical) and pyramidal tract (PT, also called cortico-subcortical) neurons.

Dimidschstein and colleagues (Nat Neurosci 19(12):1743-1749, 2016) developed a rAAV that permits largely selective gene expression in GABAergic interneurons within the telencephalon. This rAAV includes a 527 bp enhancer sequence (referred to as mI56i or mDIx) from the intergenic interval between the distal-less homeobox 5 and 6 genes (DIx5/6), which are naturally expressed by forebrain GABAergic interneurons during embryonic development. The construct of Dimidschstein et al. is available on Addgene as ID #83900 (in which the enhancer drives eGFP expression). Additional constructs which employ the murine or human I56i enhancer to drive various transgenes are available through Addgene, such as Plasmid ID #s 83899 (driving GCaMP6f expression), 83898 (driving ChR2 expression), 83895 (driving synthetic eGFP expression), 89897 (driving hM3DREADD expression), 83896 (driving hM4Di expression), and 83894 (driving synthetic tdTomato expression). See also U.S. Patent Publication No. US2018/0078658.

Additionally, the mI56i enhancer has previously been used to reliably target reporter genes in a pattern very similar to the normal patterns of DIx5/6 expression during embryonic development (Zerucha et al., J Neuroscience 20:709-721, 2000; Stühmer et al., Cerebral Cortex 12:75-85, 2002; Stenman et al., J Neuroscience 23:167-174, 2003; Monory et al., Neuron. 51:455-455, 2006; Miyoshi et al., J Neuroscience 30:1532-1594, 2010).

One significant drawback to using rAAVs as a gene-delivery system is the restricted packaging limit of AAVs; this is particularly limiting to the inclusion of lengthy genetic control and expression elements. In addition, many existing interneuron-specific rAAV expression constructs can provide weak gene expression reducing their usefulness in research and therapeutic uses.

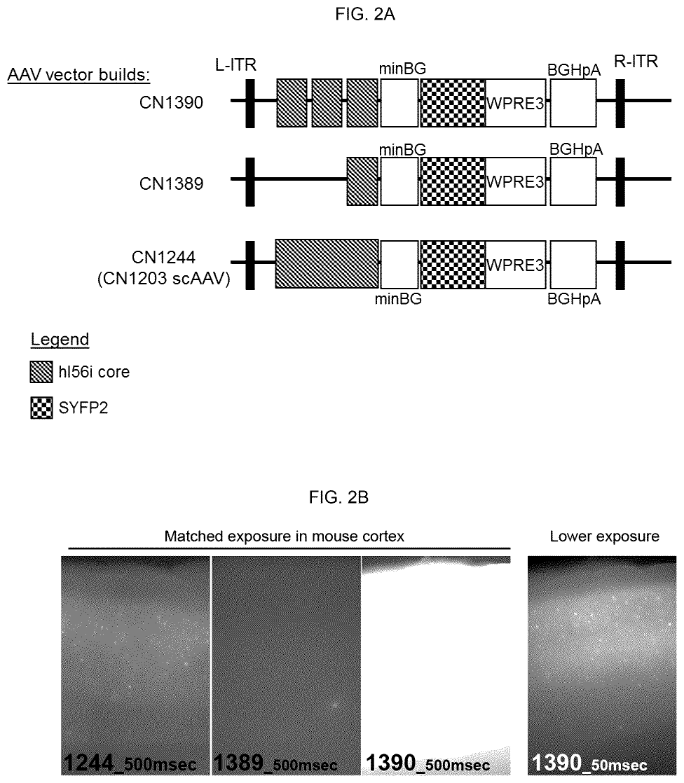

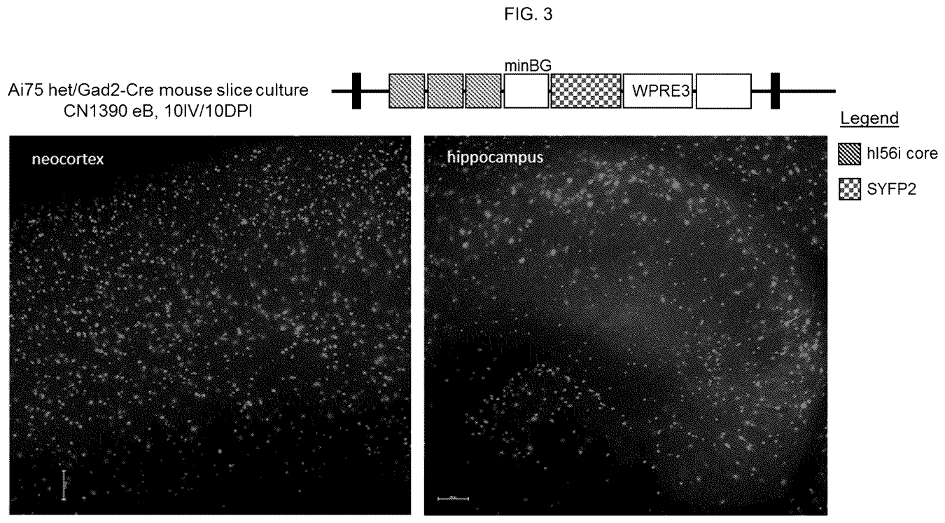

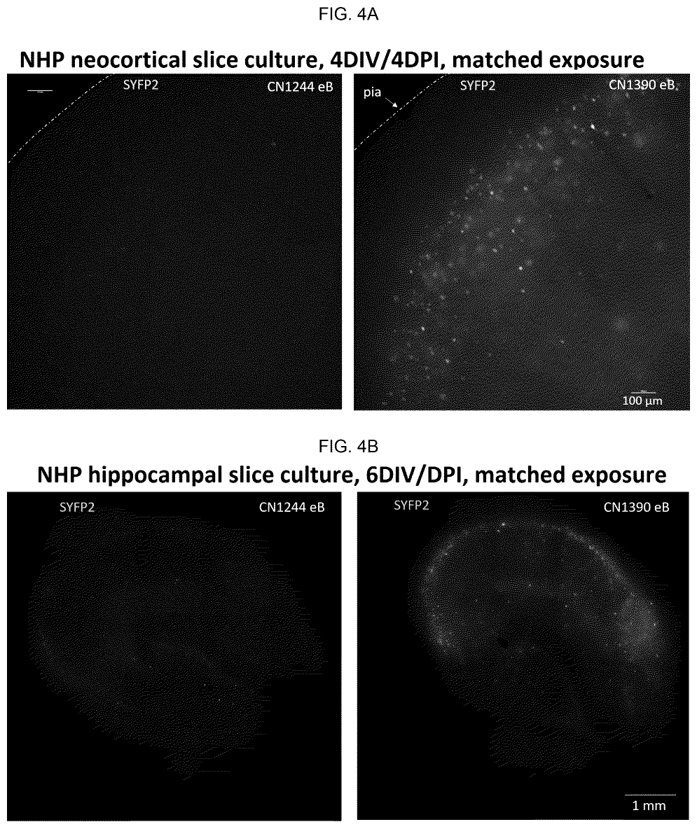

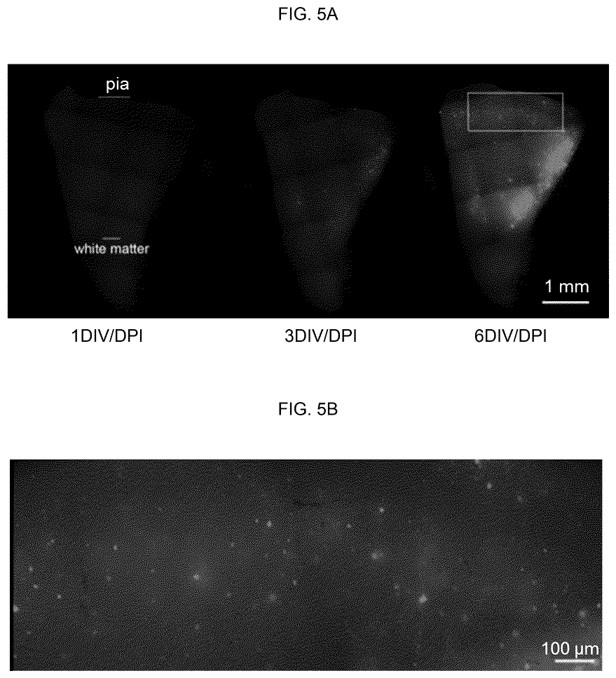

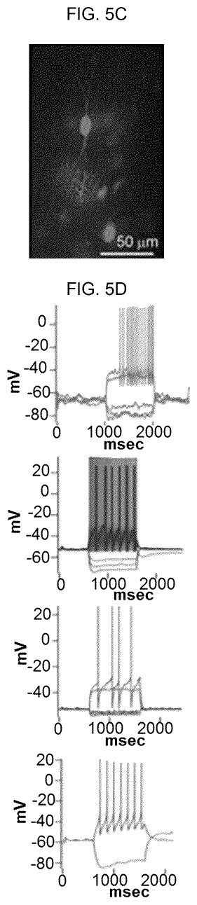

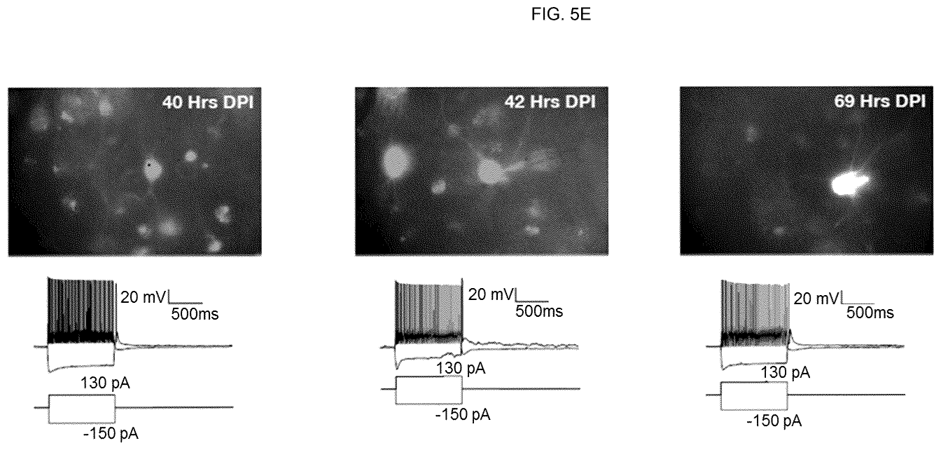

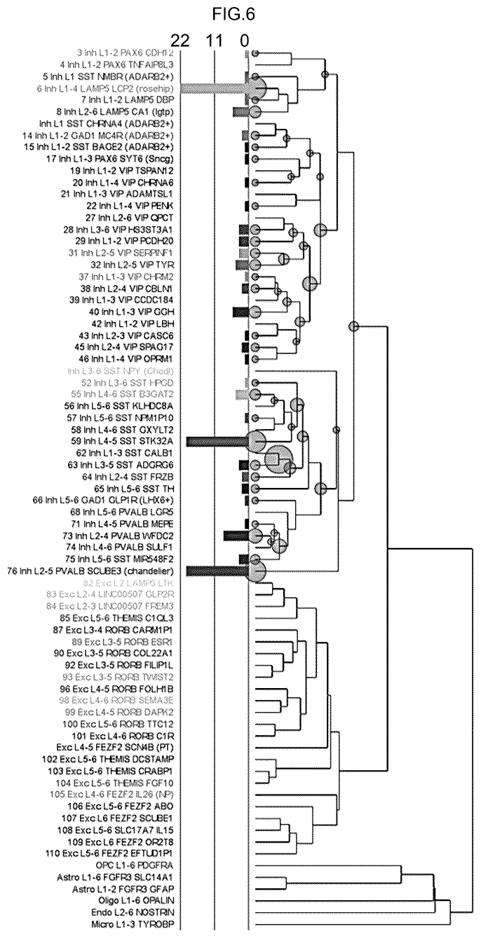

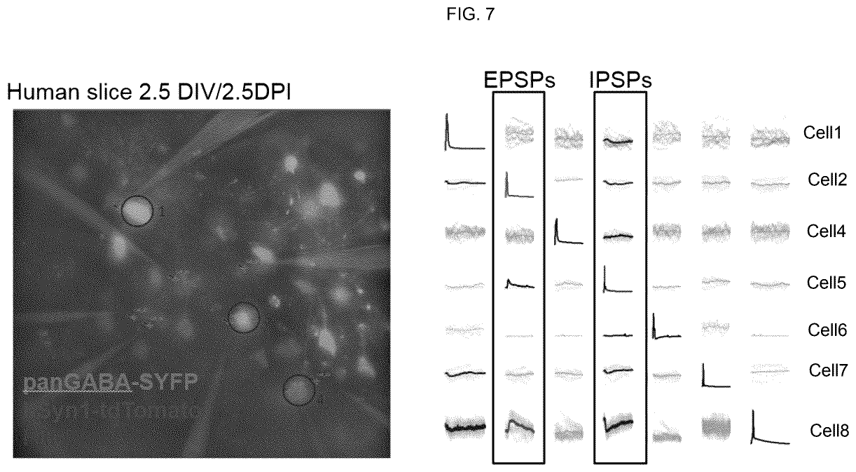

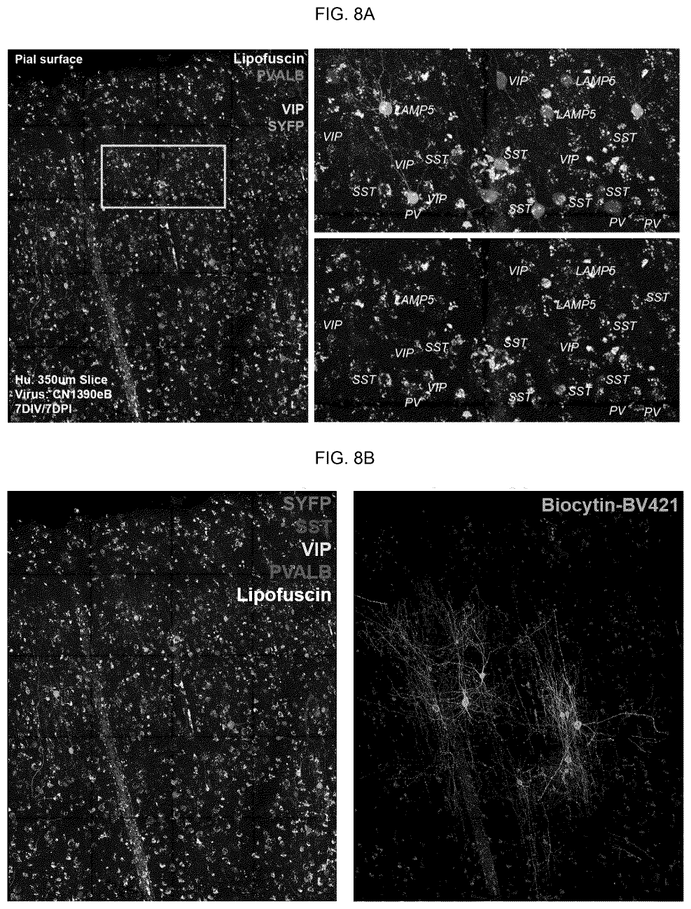

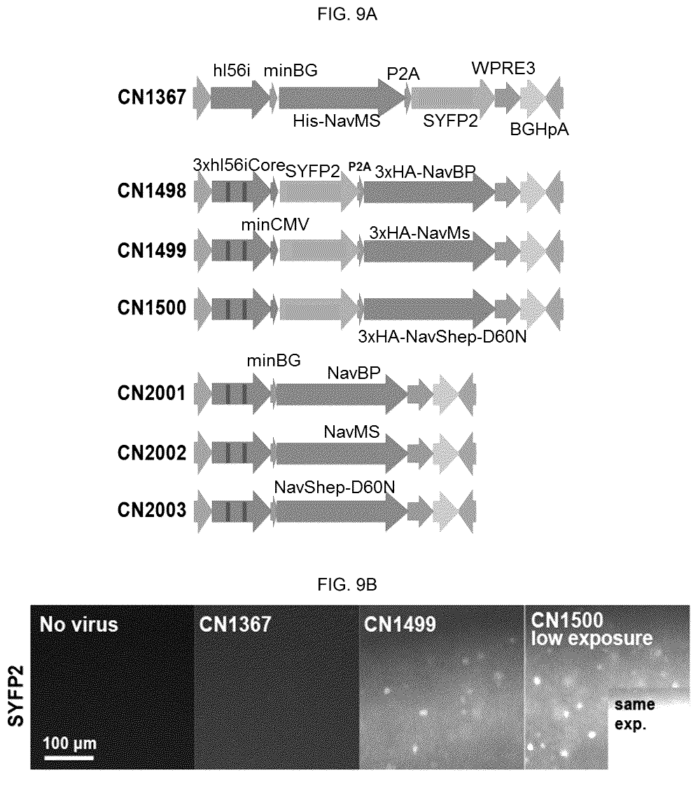

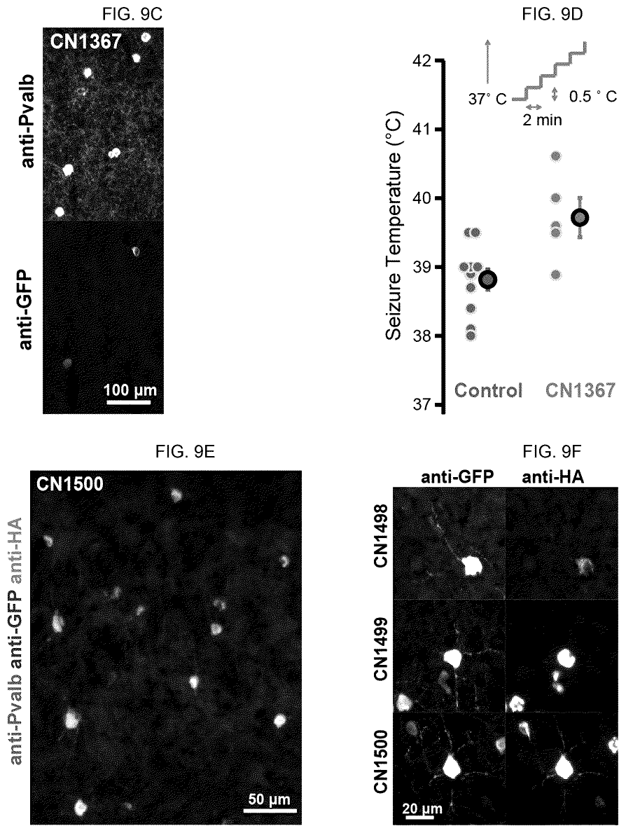

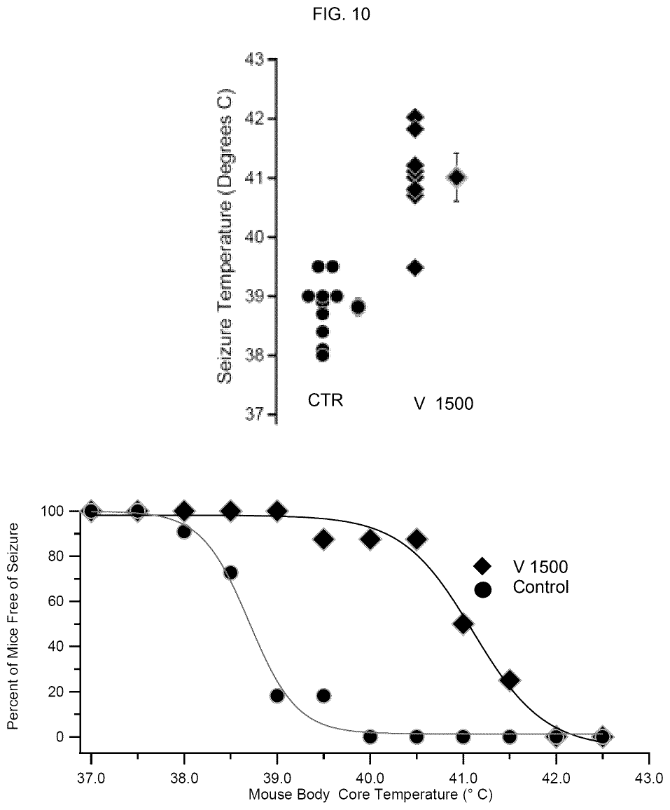

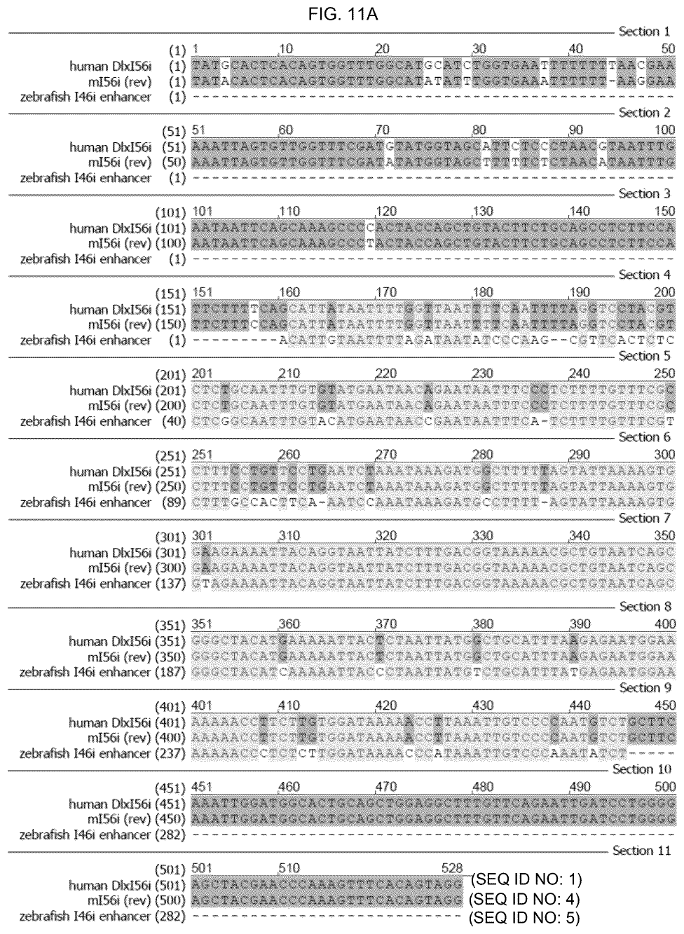

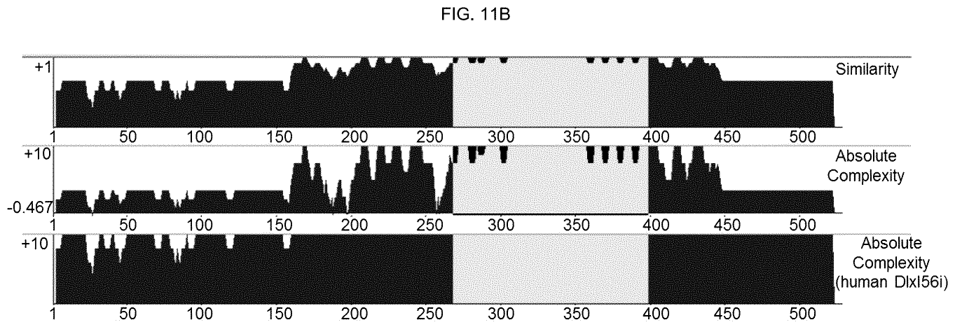

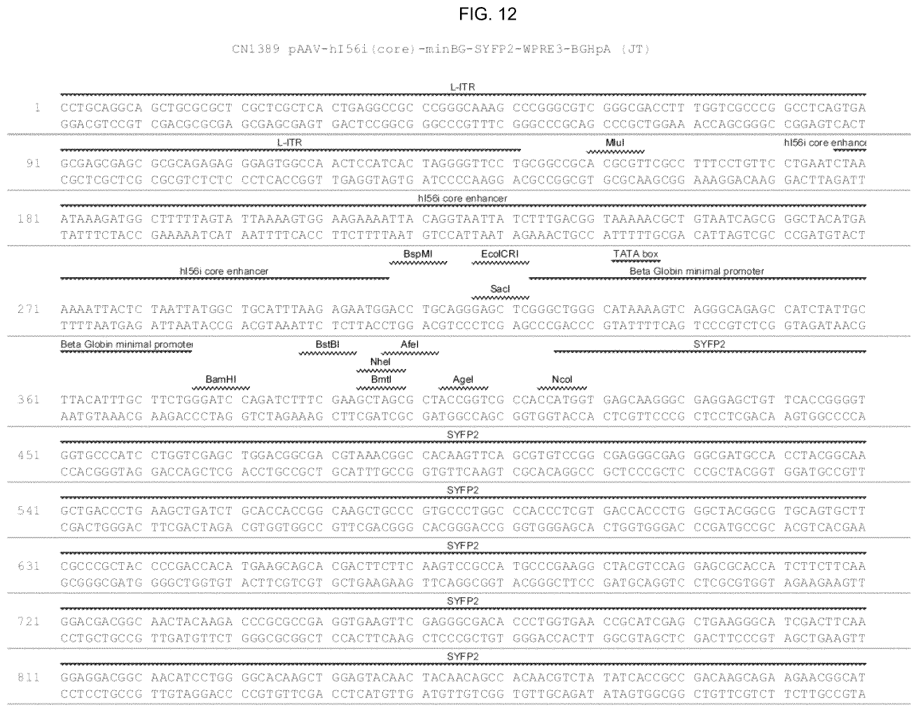

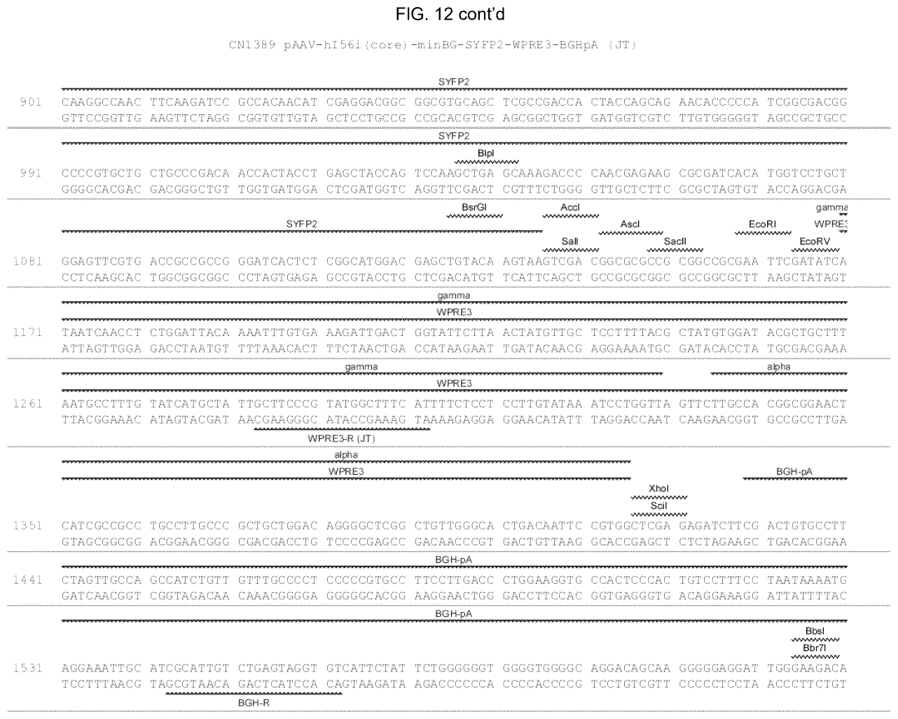

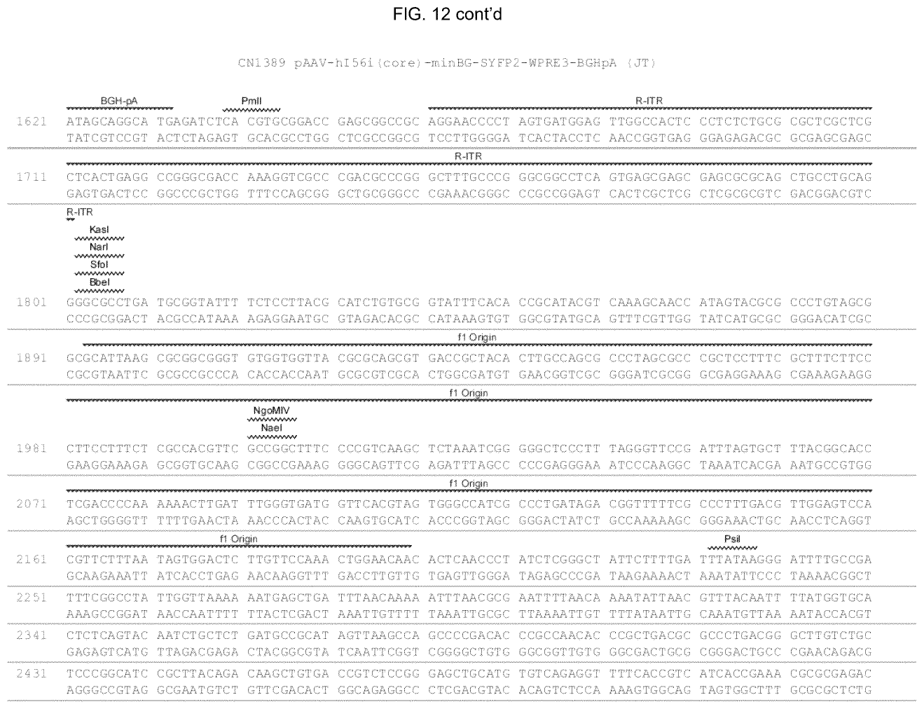

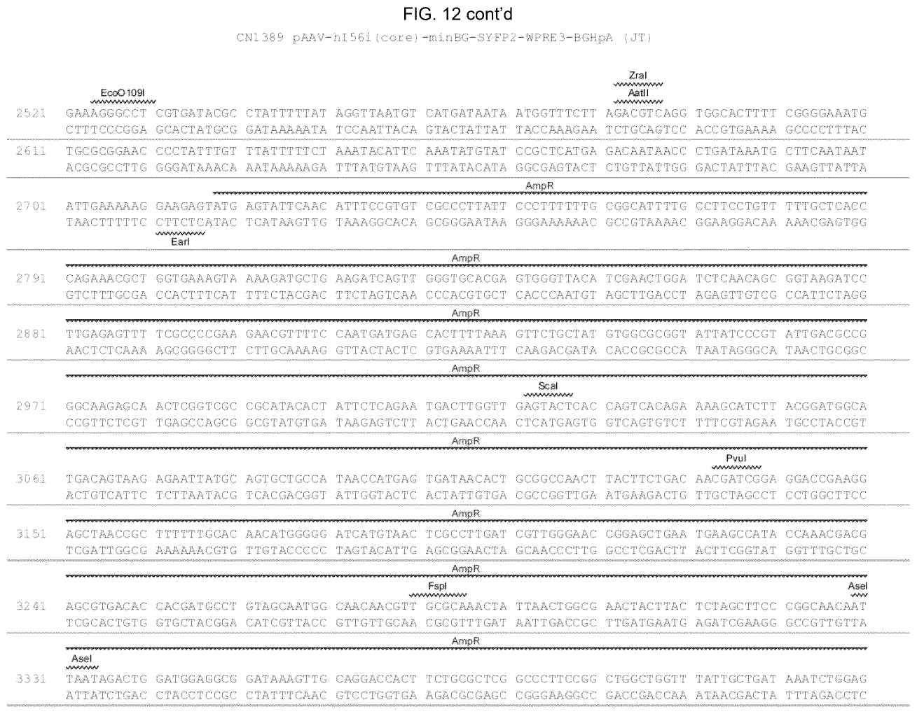

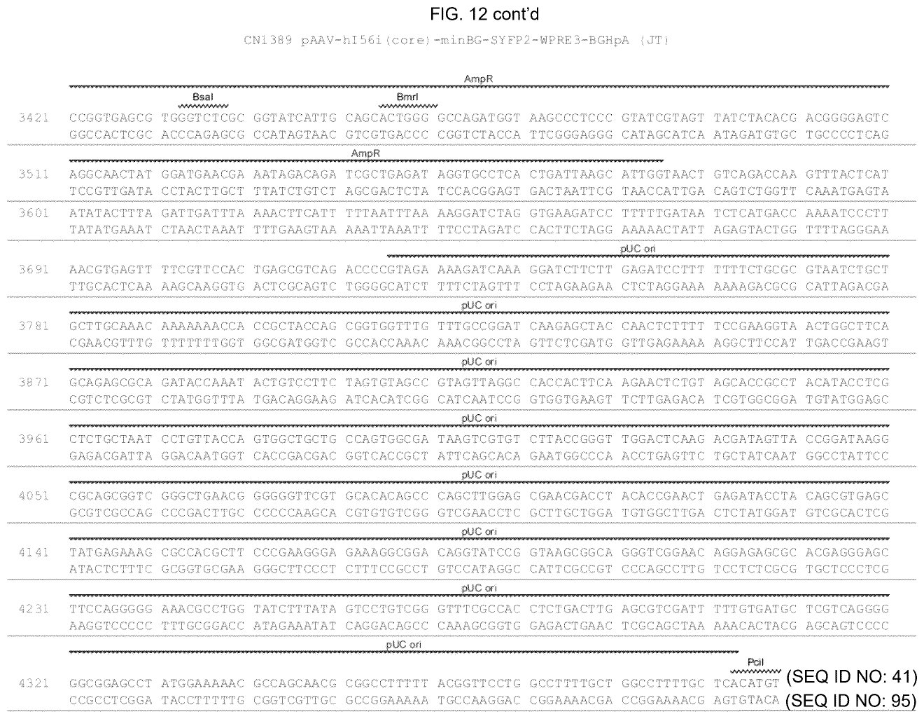

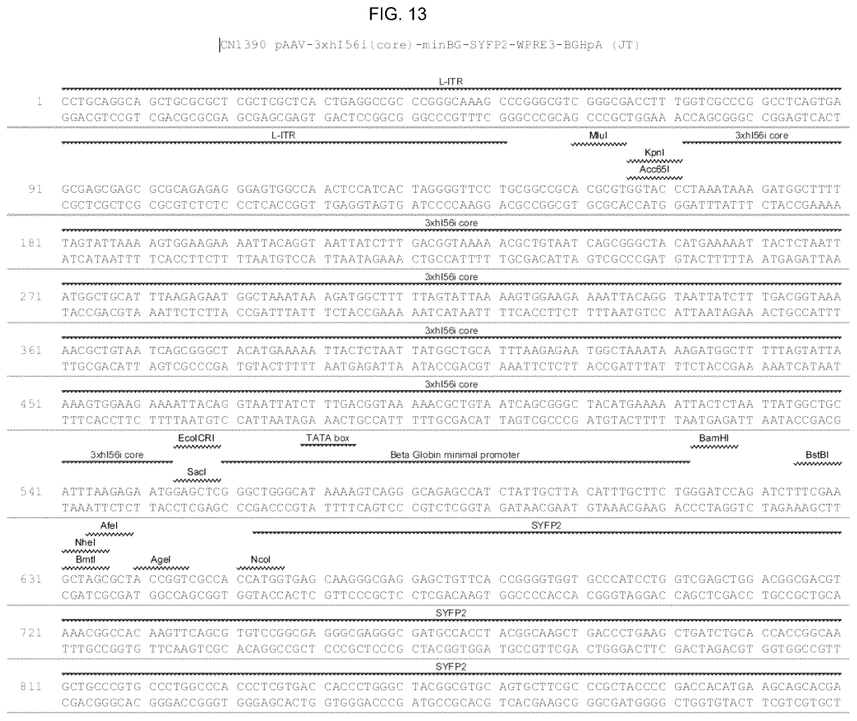

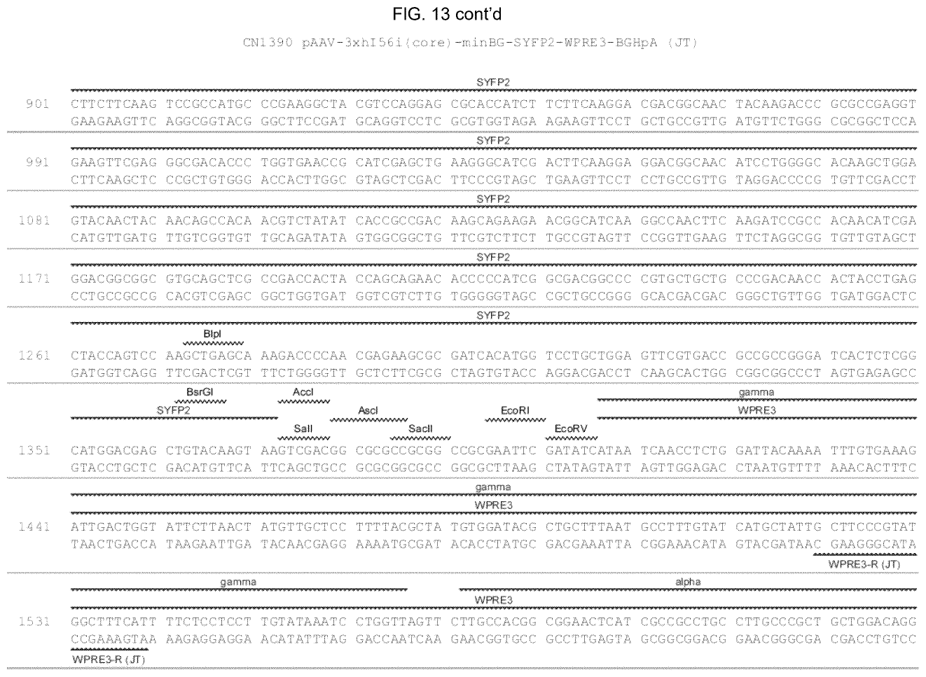

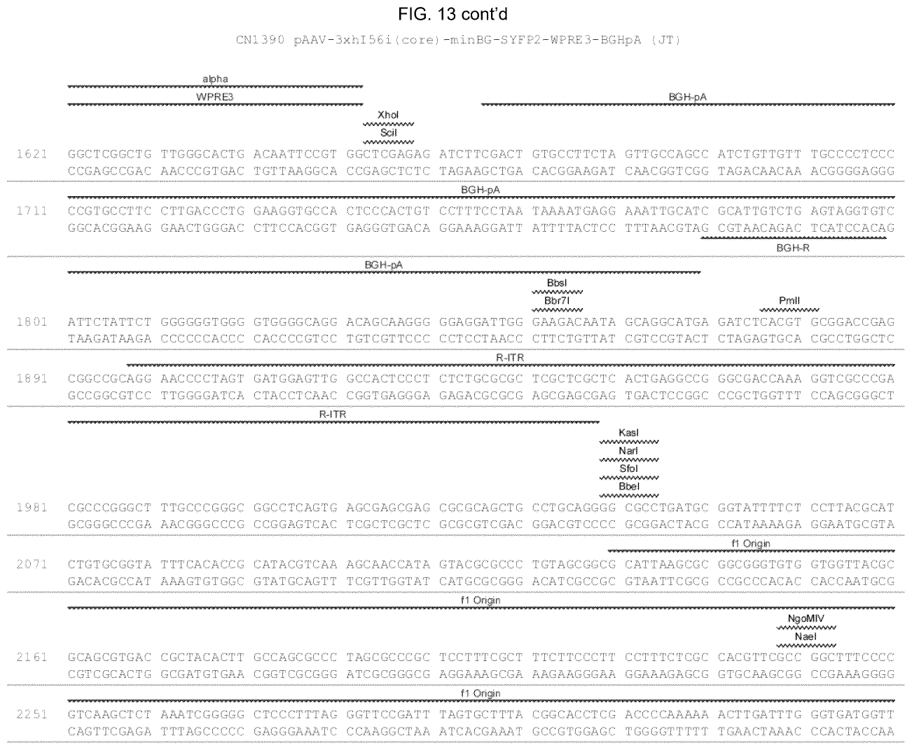

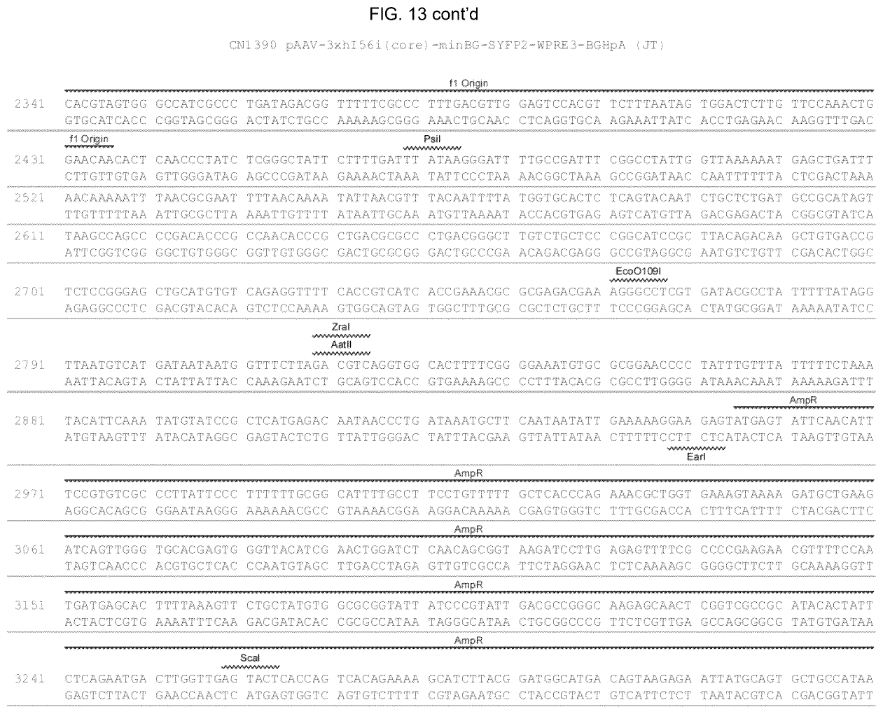

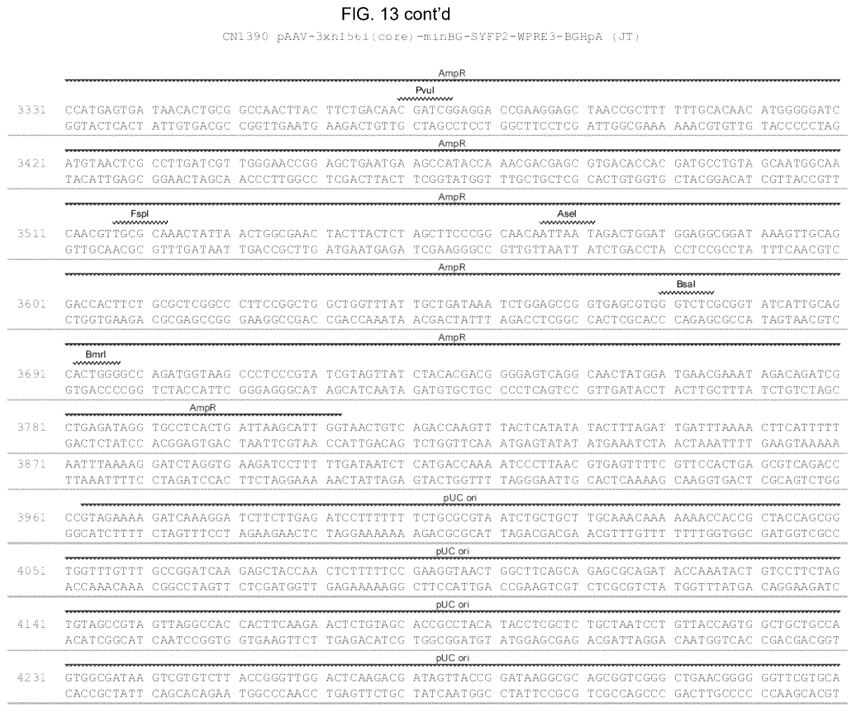

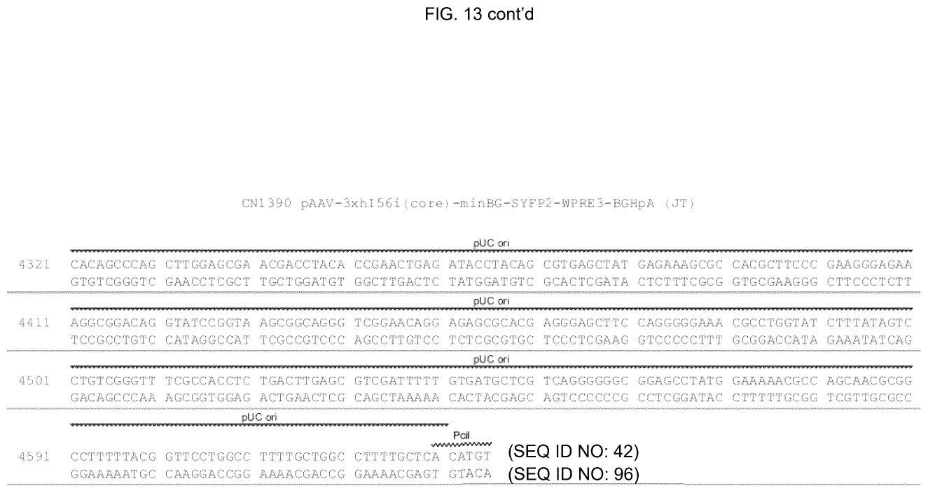

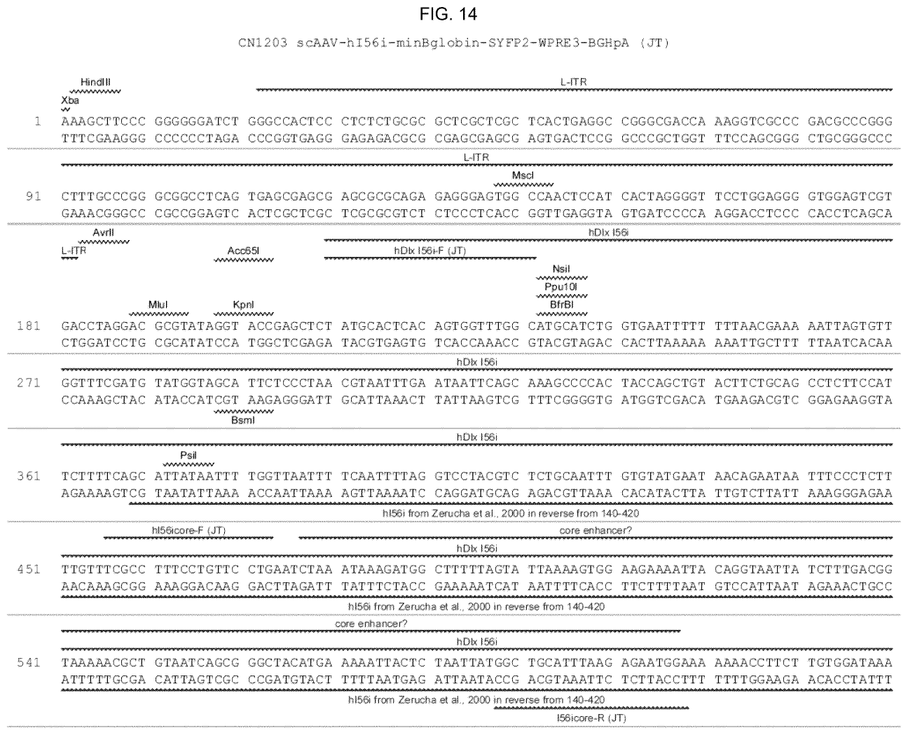

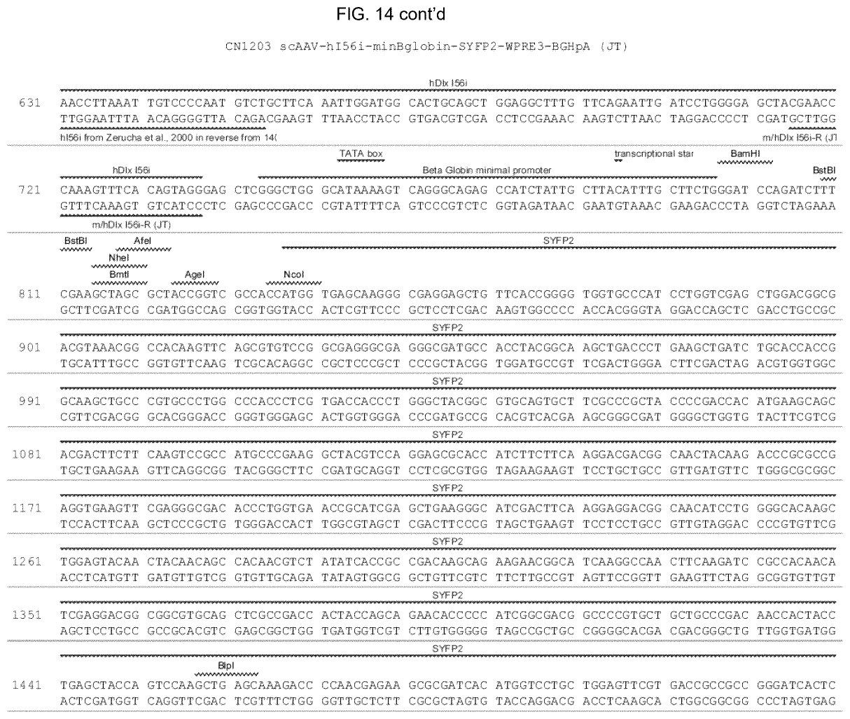

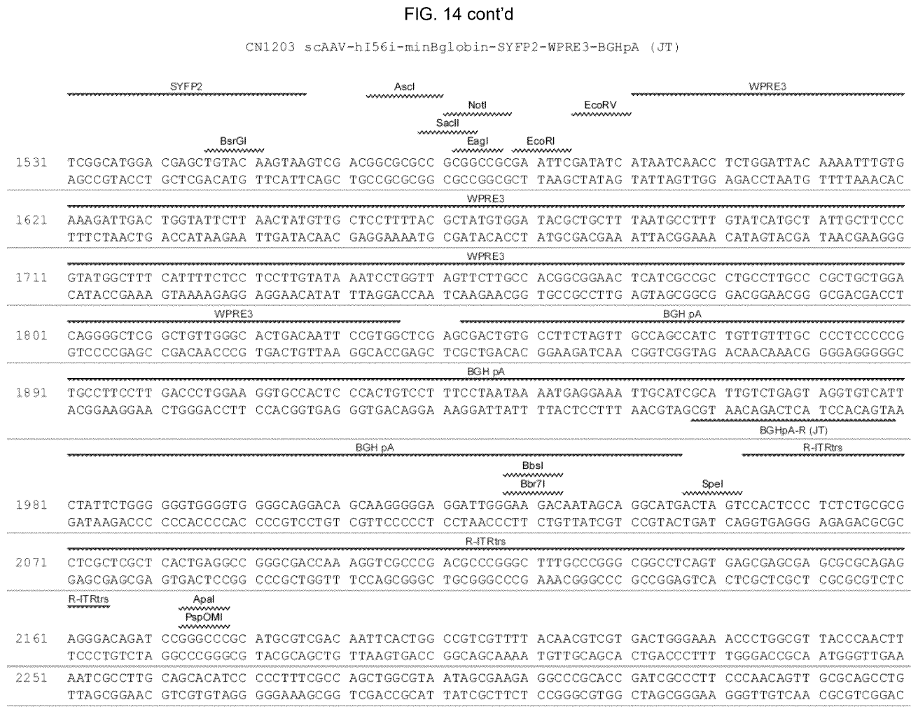

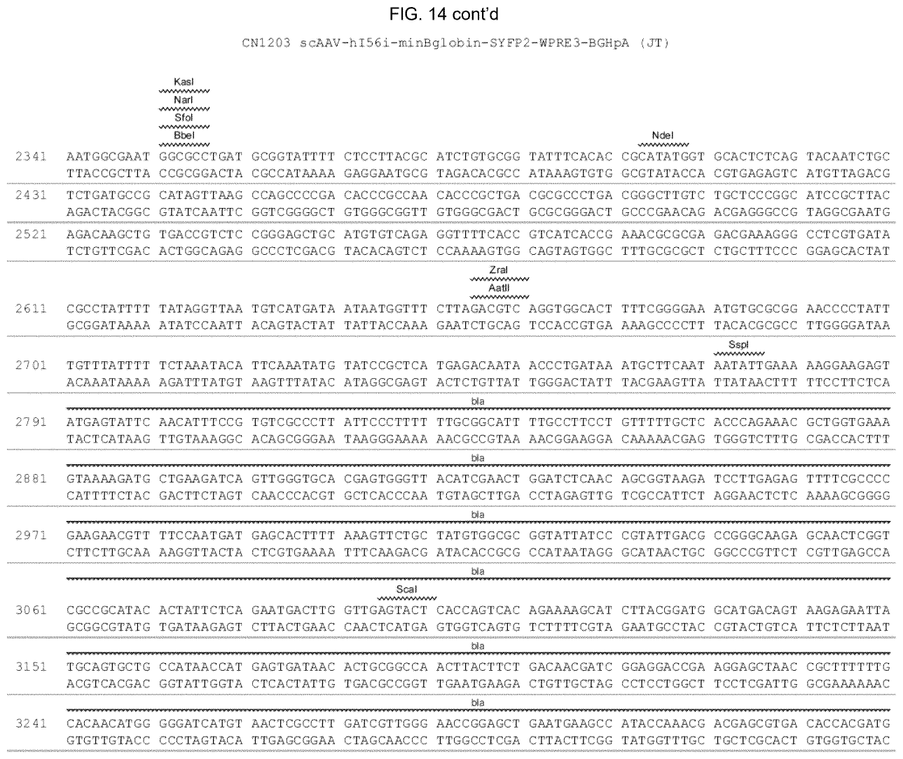

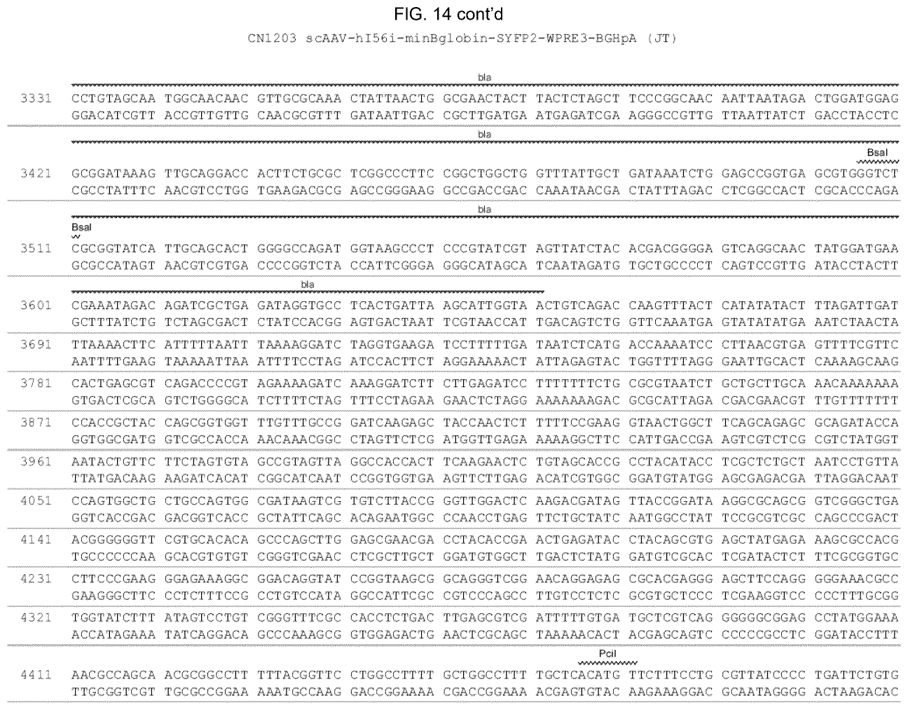

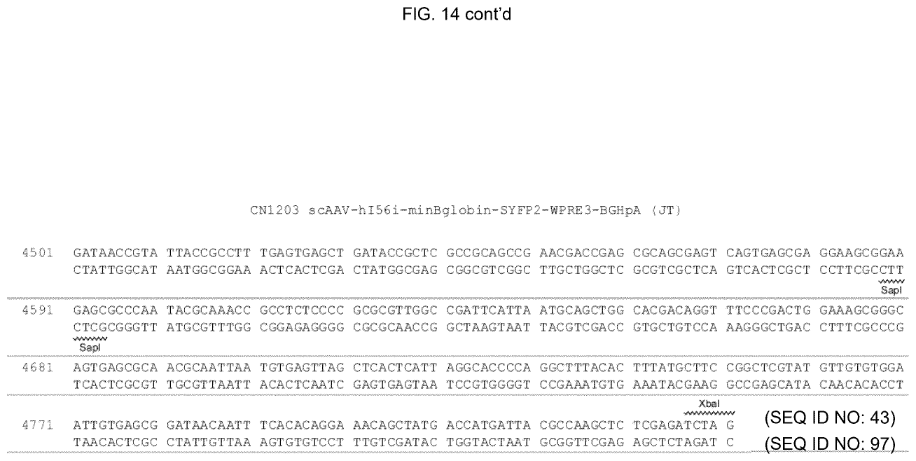

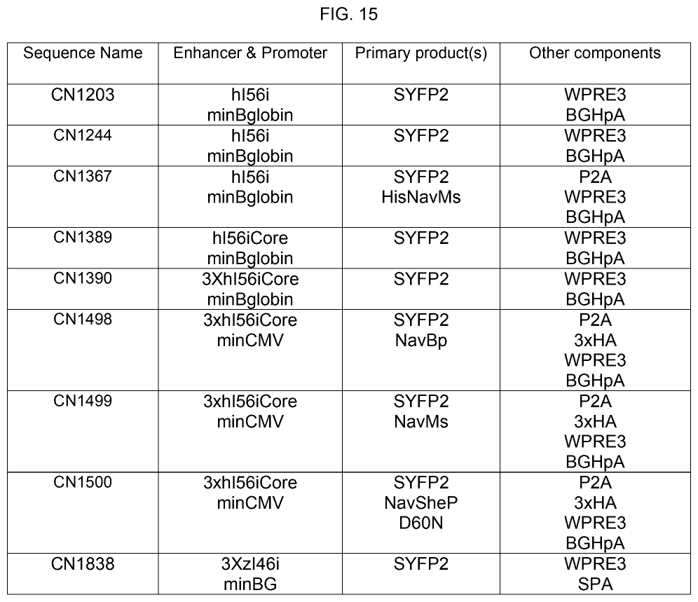

The current disclosure overcomes drawbacks of the prior art by providing artificial enhancer elements that include a concatemerized core of a I56i enhancer. These artificial enhancer elements provide unexpectedly strong peak transgene expression in forebrain GABAergic interneurons following viral transduction of mouse, monkey, and human brain tissue (see FIGS. 2A, 2B, 3, 4, 5A, 5E, 7, 8A, and 8B). The onset is also surprisingly rapid (see FIGS. 5A-5E), leading to faster and higher expression in direct comparison to virus packaged with, for instance, Addgene plasmid #83900. The increase in expression appears to be synergistically supra-linear and not simply three times the level driven by the original enhancer (FIG. 2B).

In particular embodiments, the I56i enhancer core can be derived from, for example the human and murine I56i enhancer, or zebrafish I46i enhancer (SEQ ID NOs. 1, 4, and 5 respectively). The selected cores of the I56i enhancer can include SEQ ID NO: 2 (core shared by human and mouse) or SEQ ID NO: 6 (zebrafish I46icore). In particular embodiments, the cores are concatemerized. For example, SEQ ID NO: 3 provides a three-copy concatemer of the selected human/murine I56i core while SEQ ID NO: 7 provides a three-copy concatemer of the selected zebrafish I46i core.

Of particular interest, the synthetic 3× human/murine core (referred to herein as the 3×hI56iCore; SEQ ID NO: 3) is shorter than the original full-length enhancer sequence reported in Dimidschstein et al. (Nat Neurosci 19(12):1743-1749, 2016), despite being a 3× concatemer. When used to construct a heterologous expression cassette, such as a recombinant adeno-associated virus (rAAV), this artificial enhancer element provides more room for cargo genes (heterologous encoding sequences) linked to the enhancer. This is highly desirable in many gene expression vectors. For instance, many functioning protein cargo genes (more generally, effector elements) are too long to fit in an AAV vector design, so space (length of sequence) is at a premium in the overall vector.

The engineered concatemerized I56i cores disclosed herein enable new and improved gene delivery vectors that are particularly useful for achieving selective transgene expression in neocortical GABAergic interneurons in diverse animal species, including humans and non-human primates. Importantly, GABAergic interneurons are highly involved in central processing and development and their dysfunction is implicated in a variety of brain disorders. As such, the herein-described enhancers and expression constructs have many immediate applications in research and clinical treatment development. The artificial enhancers can be used in experimental contexts where the original enhancer hI56i proved insufficient (e.g. retroorbital delivery of virus encoding transgenes for functional perturbation experiments).

Aspects of the disclosure are now described with the following additional options and detail: (i) Artificial Expression Constructs & Vectors for Selective Expression of Genes in Selected Cell Types; (ii) Compositions for Administration (iii) Cell Lines Including Artificial Expression Constructs; (iv) Transgenic Animals; (v) Methods of Use; (vi) Kits and Commercial Packages; (vii) Exemplary Embodiments; (viii) Experimental Examples; and (ix) Closing Paragraphs.

### (i) ARTIFICIAL EXPRESSION CONSTRUCTS & VECTORS FOR SELECTIVE EXPRESSION OF GENES IN SELECTED CELL TYPES

Artificial expression constructs disclosed herein include (i) an enhancer sequence that leads to selective expression of a coding sequence within a targeted central nervous system cell type, (ii) a coding sequence that is expressed, and (iii) a promoter. The expression construct can also include other regulatory elements if necessary or beneficial.

In particular embodiments, an “enhancer” or an “enhancer element” is a cis-acting sequence that increases the level of transcription associated with a promoter and can function in either orientation relative to the promoter and the coding sequence that is to be transcribed and can be located upstream or downstream relative to the promoter or the coding sequence to be transcribed. There are art-recognized methods and techniques for measuring function(s) of enhancer element sequences. Particular examples of enhancer sequences utilized within artificial expression constructs disclosed herein include concatemerized cores of I56i enhancers, such as concatamerizations of SEQ ID NO: 2 and/or 6 including, as examples, SEQ ID NO: 3 and 7. Additional particular examples of concatemerized cores of I56i enhancers can include SEQ ID NO: 2 and SEQ ID NO: 6 within one sequence such as SEQ ID NO: 2-SEQ ID NO: 2-SEQ ID NO: 6; SEQ ID NO: 2-SEQ ID NO: 6-SEQ ID NO: 6; SEQ ID NO: 2-SEQ ID NO: 6-SEQ ID NO: 2; SEQ ID NO: 6-SEQ ID NO: 6-SEQ ID NO: 2; SEQ ID NO: 6-SEQ ID NO: 2-SEQ ID NO: 2; and SEQ ID NO: 6-SEQ ID NO: 2-SEQ ID NO: 6.

In particular embodiments, a targeted central nervous system cell type enhancer is an enhancer that is uniquely or predominantly utilized by the targeted central nervous system cell type. A targeted central nervous system cell type enhancer enhances expression of a gene in the targeted central nervous system cell type but does not substantially direct expression of genes in other non-targeted cell types, thus having neural specific transcriptional activity.

When a coding sequence is selectively expressed in selected neural cells and is not substantially expressed in other neural cell types, the product of the coding sequence is preferentially expressed in the selected cell type. In particular embodiments, preferential expression is greater than 50% expression as compared to a reference cell type; greater than 60% expression as compared to a reference cell type; greater than 70% expression as compared to a reference cell type; greater than 80% expression as compared to a reference cell type; or greater than 90% expression as compared to a reference cell type. In particular embodiments, a reference cell type refers to non-targeted neural cells. The non-targeted neural cells can be within the same anatomical structure as the targeted cells and/or can project to a common anatomical area. In particular embodiments, a reference cell type is within an anatomical structure that is adjacent to an anatomical structure that includes the targeted cell type. In particular embodiments, a reference cell type is a non-targeted neural cell with a different gene expression profile than the targeted cells.

In particular embodiments, the product of the coding sequence may be expressed at low levels in non-selected cell types, for example at less than 1% or 1%, 2%, 3%, 5%, 10%, 15% or 20% of the levels at which the product is expressed in selected neural cells. In particular embodiments, the targeted central nervous system cell type is the only cell type that expresses the right combination of transcription factors that bind an enhancer disclosed herein to drive gene expression. Thus, in particular embodiments, expression occurs exclusively within the targeted cell type.

In particular embodiments, targeted cell types (e.g. neural, neuronal, and/or non-neuronal) can be identified based on transcriptional profiles, such as those described in Tasic et al., 2018 Nature. For reference, the following description of neural cell types and distinguishing features is also provided:

GABAergic Interneurons: express GABA synthesis genes Gad1/GAD1 and/or Gad2/GAD2.

### GABAergic Subclasses

Lamp5: Found in many cortical layers, especially upper (L1-L2/3), and have mainly neurogliaform and single bouquet morphology.
Sncg: Found in many cortical layers, and have molecular overlaps with Lamp5 and Vip cells, but inconsistent expression of Lamp5 or Vip, with more consistent expression of Sncg. These neurons express the neurotransmitter Cck and have primarily multipolar or basket cell morphology.
Serpinf1: Found in many cortical layers, and have molecular overlaps with Sncg and Vip cells, but inconsistent expression of Sncg or Vip, with more consistent expression of Serpinf1.
Vip: Found in many cortical layers, but especially frequent in upper layers (L1-L4), and highly express the neurotransmitter vasoactive intestinal peptide (Vip).
Sst: Found in many cortical layers, but especially frequent in lower layers (L5-L6). They highly express the neurotransmitter somatostatin (Sst), and frequently block dendritic inputs to postsynaptic neurons. Included in this subclass are sleep-active horizontal-projecting Sst Chodl (or Sst Nos1) neurons that are highly distinct from other Sst neurons, but express shared marker genes including Sst.
Pvalb: Found in many cortical layers, but especially frequent in lower layers (L5-L6). They highly express the neurotransmitter parvalbumin (Pvalb), express Tac1, and frequently dampen the output of postsynaptic neurons. Included in this subclass are chandelier cells, which have distinct, chandelier-like morphology and express the markers Cpne5 and Vipr2 in mouse, and NOG and UNC5B in human.
Meis2: A distinct subclass defined by a single type, found in L6b and subcortical white matter. Lamp5, Sncg, Serpinf1, and VIP: Developmentally derived from precursor neurons in the caudal ganglionic eminence (CGE).
Sst and Pvalb: Developmentally derived from precursor neurons in the medial ganglionic eminence (MGE).

### Glutamatergic Subclasses

All: Express glutamate transmitters Slc17a6 and/or Slc17a7.
L2/3 IT: Primarily reside in Layer 2/3 and have mainly intratelencephalic (cortico-cortical) projections.
L4 IT: Primarily reside in Layer 4 and have mainly intratelencephalic (cortico-cortical) projections.
L5 IT: Primarily reside in Layer 5 and have mainly intratelencephalic (cortico-cortical) projections. Also called L5a.
L5 PT: Primarily reside in Layer 5 and have mainly cortico-subcortical (pyramidal tract or corticofugal) projections. Also called L5b or L5 CF. These cells are located in primary motor cortex and neighboring areas and are corticospinal projection neurons. They are associated with motor neuron/movement disorders, such as ALS.
Neocortical L5 extratelencephalic (ET)-projecting pyramidal neurons (L5 ET): thick-tufted pyramidal neurons, including distinctive subtypes found only in specialized regions, e.g. Betz cells, Meynert cells, and von Economo cells.
L5 NP: Primarily reside in Layer 5 and have mainly nearby projections.
L6 CT: Primarily reside in Layer 6 and have mainly cortico-thalamic projections.
L6 IT: Primarily reside in Layer 6 and have mainly intratelencephalic (cortico-cortical) projections. Included in this subclass are L6 IT Car3 cells, which are highly similar to intracortical-projecting cells in the claustrum.
L6b: Primarily reside in the cortical subplate (L6b), with observed projections to local regions (near the cell body), cortico-cortical projections from VISp to anterior cingulate, and cortico-subcortical projections to the thalamus.
CR: A distinct subclass defined by a single type in L1, Cajal-Retzius cells express distinct molecular markers Lhx5 and Trp73.

### Non-Neuronal Subclasses

Astrocytes: Neuroectoderm-derived glial cells which express the marker Aqp4. They have a distinct star-shaped morphology and are involved in metabolic support of other cells in the brain.
Oligodendrocytes: Neuroectoderm-derived glial cells, which express the marker Sox10. This category includes oligodendrocyte precursor cells (OPCs). Oligodendrocytes are the subclass that is primarily responsible for myelination of neurons.
VLMCs: Vascular leptomeningeal cells (VLMCs) are part of the meninges that surround the outer layer of the cortex and express the marker genes Lum and Col1a1.
Pericytes: Blood vessel-associated cells, also called mural cells, that express the marker genes Konj8 and Abcc9. Pericytes wrap around endothelial cells and are important for regulation of capillary blood flow and are involved in blood-brain barrier permeability.
SMCs: Blood vessel-associated cells, also called mural cells, that express the marker gene Acta2. SMCs cover arterioles in the brain and are involved in blood-brain barrier permeability.
Endothelial: Cells that line blood vessels of the brain. Endothelial cells express the markers Tek and PDGF-β.
Macrophages: Immune cells, including macrophages, which are brain-resident macrophages, and perivascular macrophages (PVMs) that may be transitionally associated with brain tissue, or included as a biproduct of brain dissection methods.

In particular embodiments, a coding sequence is a heterologous coding sequence that encodes an effector element. An effector element is a sequence that is expressed to achieve, and that in fact achieves, an intended effect. Examples of effector elements include reporter genes/proteins and functional genes/proteins.

Exemplary reporter genes/proteins include those expressed by Addgene ID #s 83894 (pAAV-hDIx-Flex-dTomato-Fishell_7), 83895 (pAAV-hDIx-Flex-GFP-Fishell_6), 83896 (pAAV-hDIx-GiDREADD-dTomato-Fishell-5), 83898 (pAAV-mDIx-ChR2-mCherry-Fishell-3), 83899(pAAV-mDIx-GCaMP6f-Fishell-2), 83900 (pAAV-mDIx-GFP-Fishell-1), and 89897 (pcDNA3-FLAG-mTET2 (N500)). Exemplary reporter genes particularly can include those which encode an expressible fluorescent protein, or expressible biotin; blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato, dTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

GFP is composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostability and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ε), also known as its optical cross section of 9.13×10−21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

Exemplary functional molecules include functioning ion transporters, cellular trafficking proteins, enzymes, transcription factors, neurotransmitters, calcium reporters, channel rhodopsins, guide RNA, nucleases, or designer receptors exclusively activated by designer drugs (DREADDs).

Ion transporters are transmembrane proteins that mediate transport of ions across cell membranes. These transporters are pervasive throughout most cell types and important for regulating cellular excitability and homeostasis. Ion transporters participate in numerous cellular processes such as action potentials, synaptic transmission, hormone secretion, and muscle contraction. Many important biological processes in living cells involve the translocation of cations, such as calcium (Ca2+), potassium (K+), and sodium (Na+) ions, through such ion channels. In particular embodmients, ion transporters include voltage gated sodium channels (e.g., SCN1A), potassium channels (e.g., KCNQ2), and calcium channels (e.g. CACNA1C)).

Exemplary enzymes, transcription factors, receptors, membrane proteins, cellular trafficking proteins, signaling molecules, and neurotransmitters include enzymes such as lactase, lipase, helicase, alpha-glucosidase, amylase; transcription factors such as SP1, AP-1, Heat shock factor protein 1, C/EBP (CCAA-T/enhancer binding protein), and Oct-1; receptors such as transforming growth factor receptor beta 1, platelet-derived growth factor receptor, epidermal growth factor receptor, vascular endothelial growth factor receptor, and interleukin 8 receptor alpha; membrane proteins, cellular trafficking proteins such as clathrin, dynamin, caveolin, Rab-4A, and Rab-11A; signaling molecules such as nerve growth factor (NGF), platelet-derived growth factor (PDGF), transforming growth factor β (TGFβ), epidermal growth factor (EGF), GTPase and HRas; and neurotransmitters such as cocaine and amphetamine regulated transcript, substance P, oxytocin, and somatostatin.

In particular embodmients, functional molecules include reporters of neural function and states such as calcium reporters. Intracellular calcium concentration is an important predictor of numerous cellular activities, which include neuronal activation, muscle cell contraction and second messenger signaling. A sensitive and convenient technique to monitor the intracellular calcium levels is through the genetically encoded calcium indicator (GECI). Among the GECIs, green fluorescent protein (GFP) based calcium sensors named GCaMPs are efficient and widely used tools. The GCaMPs are formed by fusion of M13 and calmodulin protein to N- and C-termini of circularly permutated GFP. Some GCaMPs yield distinct fluorescence emission spectra (Zhao et al.,Science, 2011, 333 (6051): 1888-1891). Exemplary GECIs with green fluorescence include GCaMP3, GCaMP5G, GCaMP6s, GCaMP6m, GCaMP6f, jGCaMP7s, jGCaMP7c, jGCaMP7b, andjGCaMP7f. Furthermore, GECIs with red fluorescence include jRGECO1a and jRGECO1b. AAV products containing GECIs are commercially available. For example, Vigene Biosciences provides AAV products including AAV8-CAG-GCaMP3 (Cat. No: BS4-CX3AAV8), AAV8-Syn-FLEX-GCaMP6s-WPRE (Cat. No: BS1-NXSAAV8), AAV8-Syn-FLEX-GCaMP6s-WPRE (Cat. No: BS1-NXSAAV8), AAV9-CAG-FLEX-GCaMP6m-WPRE (Cat. No: BS2-CXMAAV9), AAV9-Syn-FLEX-jGCaMP7s-WPRE (Cat. No: BS12-NXSAAV9), AAV9-CAG-FLEX-jGCaMP7f-WPRE (Cat. No: BS12-CXFAAV9), AAV9-Syn-FLEX-jGCaMP7b-WPRE (Cat. No: BS12-NXBAAV9), AAV9-Syn-FLEX-jGCaMP7c-WPRE (Cat. No: BS12-NXCAAV9), AAV9-Syn-FLEX-NES-jRGECO1a-WPRE (Cat. No: BS8-NXAAAV9), and AAV8-Syn-FLEX-NES-jRCaMP1b-WPRE (Cat. No: BS7-NXBAAV8).

In particular embodiments calcium reporters include the genetically encoded calcium indicators GECI, NTnC; Myosin light chain kinase, GFP, Calmodulin chimera; Calcium indicator TN-XXL; BRET-based auto-luminescent calcium indicator; and/or Calcium indicator protein OeNL (Ca2+)-18u).

In particular embodmients, functional molecules include modulators of neuronal activity like channel rhodopsins (e.g., channelopsin-1, channelrhodopsin-2, and variants thereof). Channelrhodopsins are a subfamily of retinylidene proteins (rhodopsins) that function as light-gated ion channels. In addition to channelrhodopsin 1 (ChR1) and channelrhodopsin 2 (ChR2), several variants of channelrhodopsins have been developed. For example, Lin et al. (Biophys J, 2009, 96(5):1803-14) describe making chimeras of the transmembrane domains of ChR1 and ChR2, combined with site-directed mutagenesis. Zhang et al. (Nat Neurosci, 2008, 11 (6): 631-3) describe VChR1, which is a red-shifted channelrhodopsin variant. VChR1 has lower light sensitivity and poor membrane trafficking and expression. Other known channelrhodopsin variants include the ChR2 variant described in Nagel, et al., Proc Natl Acad Sci USA, 2003, 100 (24): 13940-5), ChR2/H134R (Nagel, G., et al., Curr Biol, 2005, 15 (24): 2279-84), and ChD/ChEF/ChIEF (Lin, J. Y., et al., Biophys J, 2009, 96 (5): 1803-14), which are activated by blue light (470 nm) but show no sensitivity to orange/red light. Additional variants are described in Lin, Experimental Physiology, 2010, 96.1:19-25 and Knopfel et al., The Journal of Neuroscience, 2010, 30(45):14998-15004).

In particular embodmients, functional molecules include DNA and RNA editing tools such CRISPR/CAS (e.g., guide RNA and a nuclease, such as Cas, Cas9 or cpf1). Functional molecules can also include engineered Cpf1s such as those described in US 2018/0030425, US 2016/0208243, WO/2017/184768 and Zetsche et al. (2015) Cell 163:759-771; single gRNA (see e.g., Jinek et al. (2012) Science 337:816-821; Jinek et al. (2013) eLife 2: e00471; Segal (2013) eLife 2: e00563) or editase, guide RNA molecules or homologous recombination donor cassettes.

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

In particular embodmients, functional molecules include designer receptor exclusively activated by designer drug (DREADD). Designer receptors exclusively activated by designer drugs (DREADDs) can be used to modulate cellular functions (Rogan and Roth, Pharmacol. Rev. 2011, 63(2):291-315). This family of evolved muscarinic receptors has been shown to increase (Gs-DREADD; Gq-DREADD) or decrease (Gi/o-DREADD) cellular activity following administration of an otherwise inert synthetic ligand, clozapine-n-oxide (Armbruster et al., PNAS, 2007, 104(12): 5163-5168). When packaged into viral vectors or expressed in transgenic mouse models, these tools allow cellular activity to be controlled in a defined spatial and temporal manner. For example, activation of hippocampal neurons by Gq-DREADD receptors amplifies γ-rhythms and increases locomotor activity and stereotypy in mice (Alexander et al., Neuron, 2009, 63(1):27-39). DREADDs are formed by point mutations in the third and fifth transmembrane regions of muscarinic receptors (Y149C and A239G in hM3). In addition, the Gs-coupled DREADD contains the second and third intracellular loops of the β1-AR in place of those of the M3 muscarinic receptor. Some exemplary DREADDs include hM3DREADD (hM3D) and hM4DREADD (hM4D). Various plasmids containing DREADDs are commercially available. For example, on addgene, AAV plasmids containing DREADDs include: pAAV-hSyn-DIO-hM3D (Gq)-mCherry (Plasmid #44361), pAAV-hSyn-DIO-hM4d (Gi)-mCherry) (Plasmid #44362), pAAV-EF1a-DIO-hM4d (Gi)-mCherry) (Plasmid #50461), pAAV-GFAP-HA-hM3D (Gq)-IRES)-mCitrine (Plasmid #50470), and pAAV-CaMKIIa-hM4D (Gi)-mCherry (Plasmid #50477).

Additional effector elements include Cre, iCre, dgCre, FlpO, and tTA2. iCre refers to a codon-improved Cre. dgCre refers to an enhanced GFP/Cre recombinase fusion gene with an N terminal fusion of the first 159 amino acids of the Escherichia coli K-12 strain chromosomal dihydrofolate reductase gene (DHFR or folA) harboring a G67S mutation and modified to also include the R12Y/Y1001 destabilizing domain mutation. FlpO refers to a codon-optimized form of FLPe that greatly increases protein expression and FRT recombination efficiency in mouse cells. Like the Cre/LoxP system, the FLP/FRT system has been widely used for gene expression (and generating conditional knockout mice, mediated by the FLP/FRT system). tTA2 refers to tetracycline transactivator.

Exemplary expressible elements are expression products that do not include effector elements, for example, a non-functioning or defective protein. In particular embodiments, expressible elements can provide methods to study the effects of their functioning counterparts. In particular embodiments, expressible elements are non-functioning or defective based on an engineered mutation that renders them non-functioning. In these aspects, non-expressible elements are as similar in structure as possible to their functioning counterparts.

Exemplary self-cleaving peptides include the 2A peptides which lead to the production of two proteins from one mRNA. The 2A sequences are short (e.g., 20 amino acids), allowing more use in size-limited constructs. Particular examples include P2A, T2A, E2A, and F2A. In particular embodiments, the expression constructs include an internal ribosome entry site (IRES) sequence. IRES allow ribosomes to initiate translation at a second internal site on a mRNA molecule, leading to production of two proteins from one mRNA.

Coding sequences encoding molecules (e.g., RNA, proteins) described herein can be readily obtained from publicly available databases and publications. Coding sequences can further include various sequence polymorphisms, mutations, and/or sequence variants wherein such alterations do not affect the function of the encoded molecule. The term “encode” or “encoding” refers to a property of sequences of nucleic acids, such as a vector, a plasmid, a gene, cDNA, mRNA, to serve as templates for synthesis of other molecules such as proteins.

The term “gene” may include not only coding sequences but also regulatory regions such as promoters, enhancers, and termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include minBglobin, CMV, minCMV, a mutated minCMV, SV40 immediately early promoter, the Hsp68 minimal promoter (proHSP68), and the Rous Sarcoma Virus (RSV) long-terminal repeat (LTR) promoter. Minimal promoters have no activity to drive gene expression on their own, but can be activated to drive gene expression when linked to a proximal enhancer element.

In particular embodiments, expression constructs are provided within vectors. The term vector refers to a nucleic acid molecule capable of transferring or transporting another nucleic acid molecule, such as an expression construct. The transferred nucleic acid is generally linked to, e.g., inserted into, the vector nucleic acid molecule. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors.

Viral vector is widely used to refer to a nucleic acid molecule that includes virus-derived nucleic acid elements that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adenovirus. “Adenovirus vectors” refer to those constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in selected cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retrovirus. Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In particular embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In particular embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly (A) tract. The R region is also defined as being flanked by the U3 and U5 regions. The R region plays a role during reverse transcription in permitting the transfer of nascent DNA from one end of the genome to the other.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al., 1999, J. Virol., 73:2886); the posttranscriptional regulatory element present in hepatitis B virus (HPRE) (Smith et al., Nucleic Acids Res. 26(21):4818-4827, 1998); and the like (Liu et al., 1995, Genes Dev., 9:1766). In particular embodiments, vectors include a posttranscriptional regulatory element such as a WPRE or HPRE. In particular embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation sequence 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40pA. In particular embodiments, a preferred embodiment of an expression construct includes a terminator element. These elements can serve to enhance transcript levels and to minimize read through from the construct into other plasmid sequences.

In particular embodiments, a viral vector further includes one or more insulator elements. Insulators elements may contribute to protecting viral vector-expressed sequences, e.g., effector elements or expressible elements, from integration site effects, which may be mediated by cis-acting elements present in genomic DNA and lead to deregulated expression of transferred sequences (i.e., position effect; see, e.g., Burgess-Beusse et al., PNAS., USA, 99:16433, 2002; and Zhan et al., Hum. Genet., 109:471, 2001). In particular embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken β-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33:198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Particular embodiments of vectors disclosed herein include:

| Expression | |
|---|---|
| Construct | |
| Name | Features |
| CN1390 | rAAV: 3xhl56Core-minBglobin-SYFP2-WPRE3-BGHpA |
| CN1244 | rAAV: hl56i-minBglobin-SYFP2-WPRE3-BGHpA |
| CN1389 | rAAV: hl56Core-minBglobin-SYFP2-WPRE3-BGHpA |
| CN1203 | scAAV: hl56i-minBglobin-SYFP2-WPRE3-BGHpA |
| CN1367 | rAAV: hl56i-minBglobin-His-NavMs-P2A-SYFP2-WPRE3-BGHpA |
| CN1498 | rAAV: 3xhl56iCore-minCMV-SYFP2-P2A-3xHA-NavBp-WPRE3-BGHpA |
| CN1499 | rAAV: 3xhl56iCore-minCMV-SYFP2-P2A-3xHA-NavMs-WPRE3-BGHpA |
| CN1500 | rAAV: 3xhl56iCore-minCMV-SYFP2-P2A-3xHA-NavSheP-D60N-WPRE3- |
| | BGHpA |
| CN1838 | scAAV-3Xzl46i-minBG-SYFP2-WPRE3-SPA |

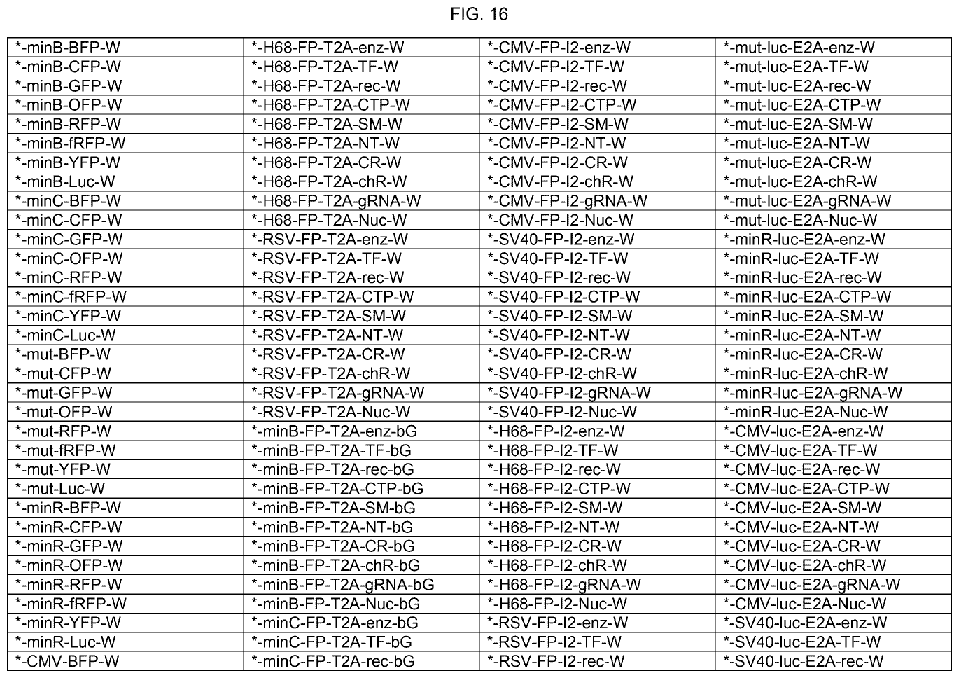

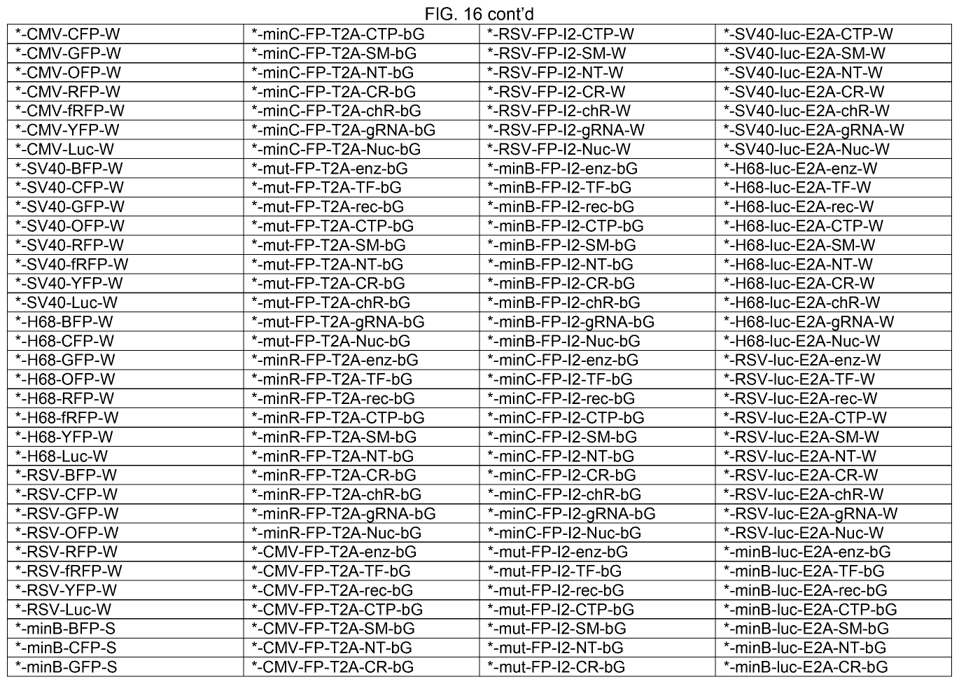

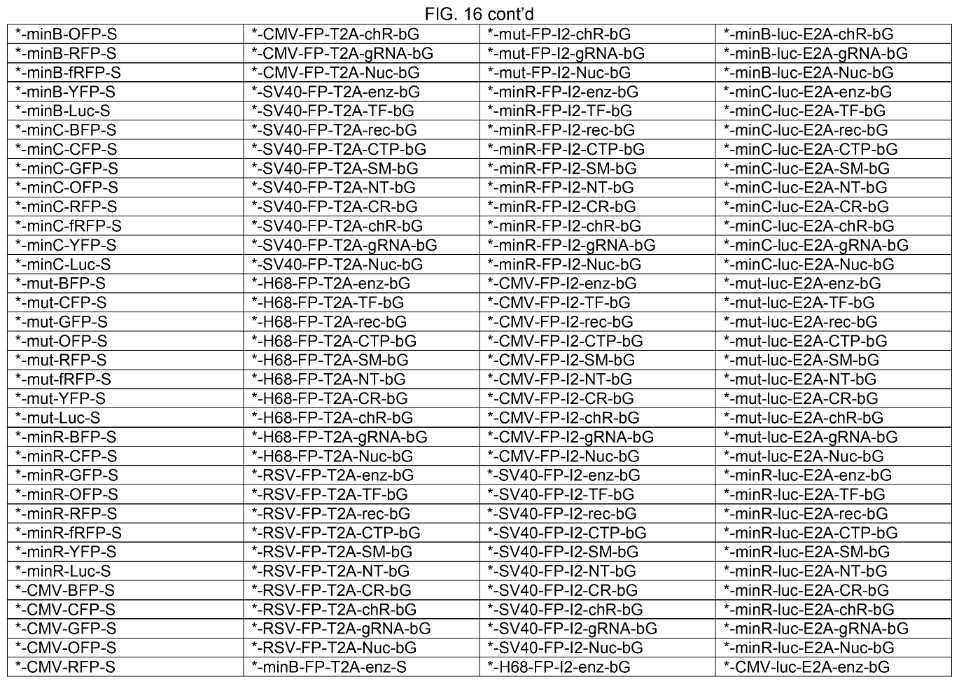

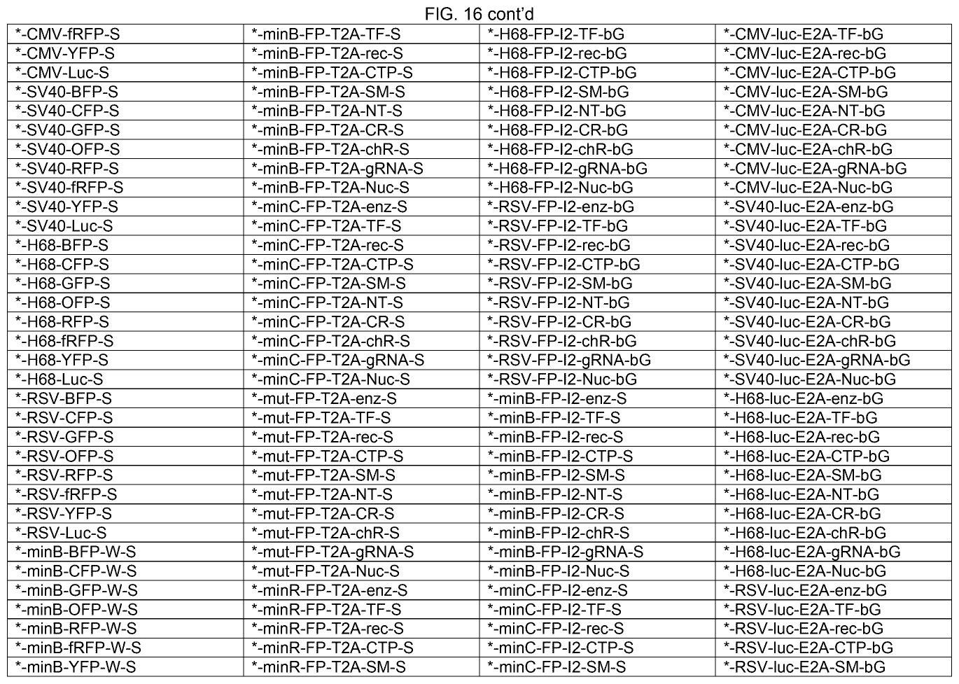

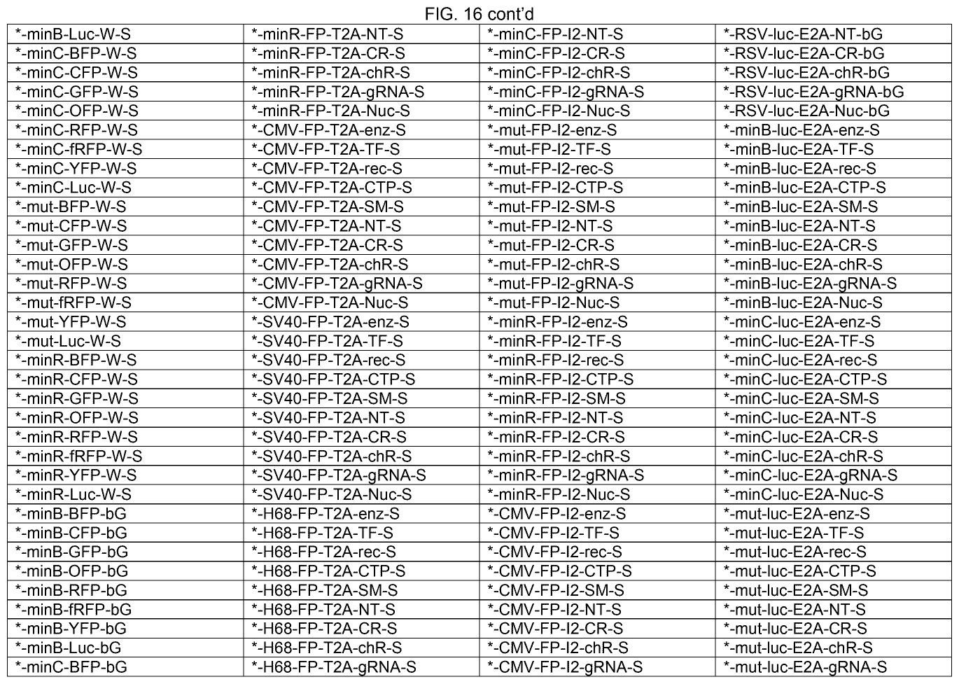

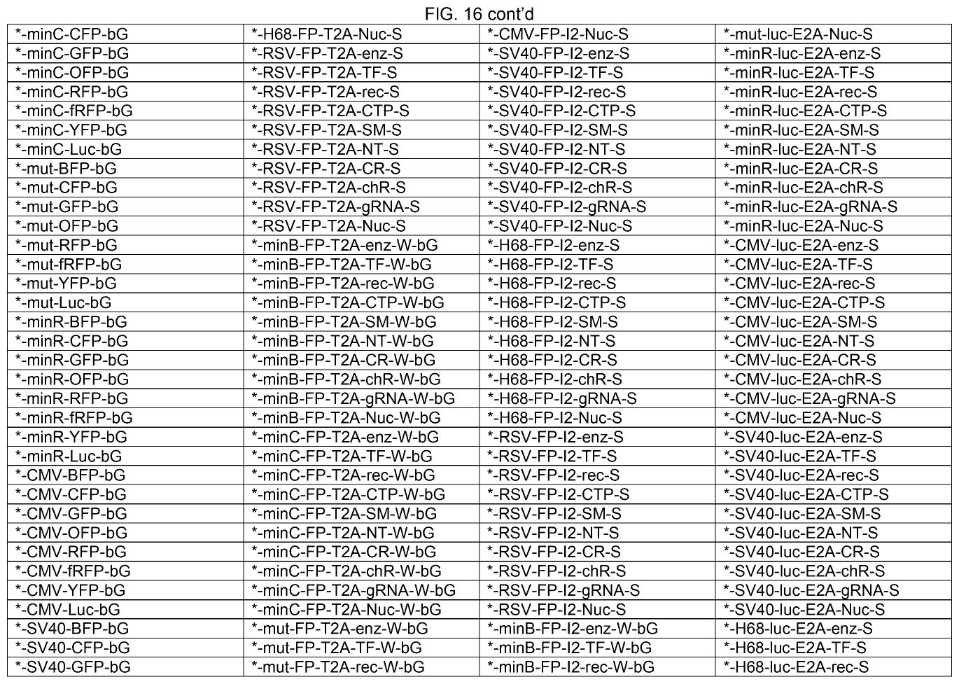

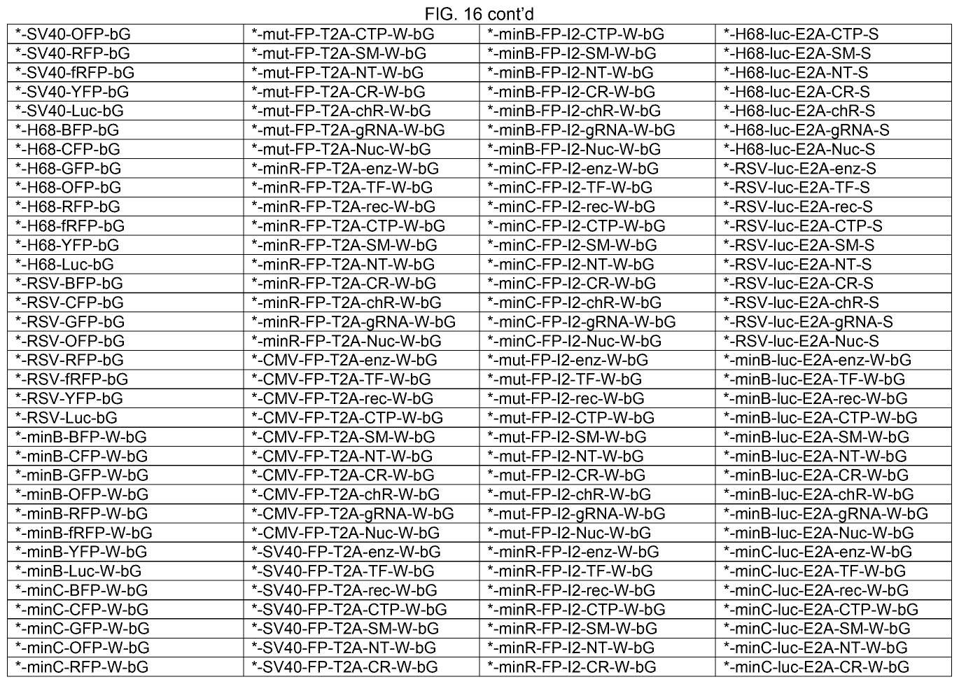

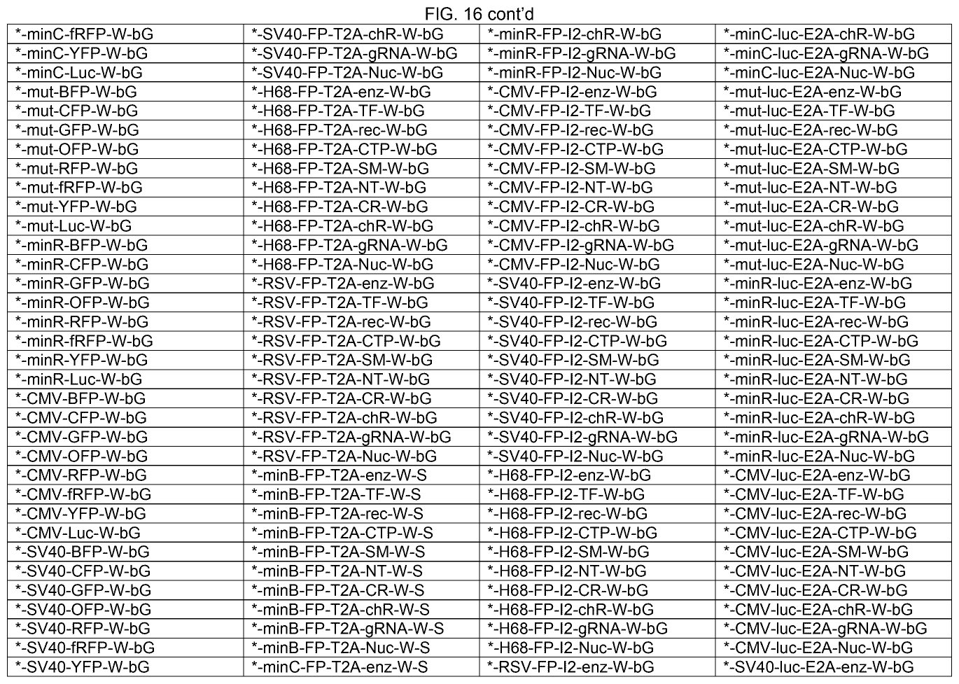

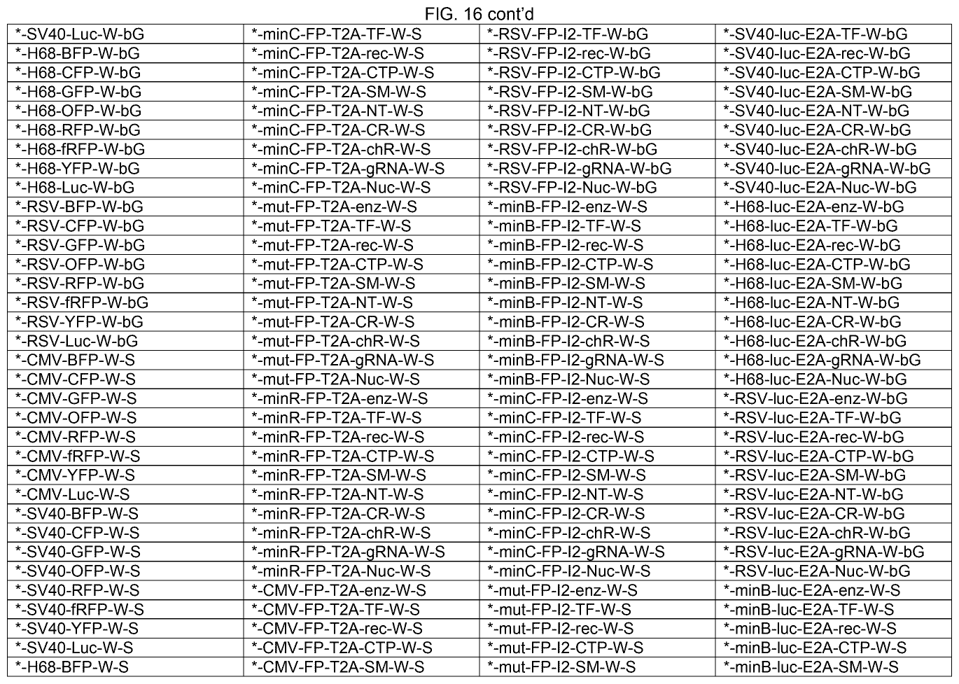

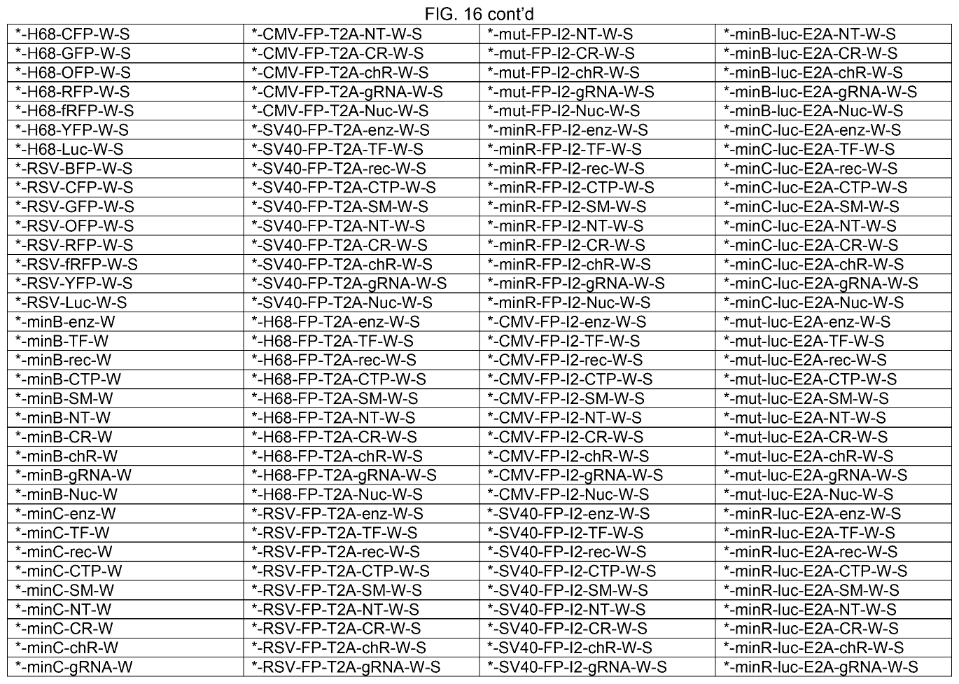

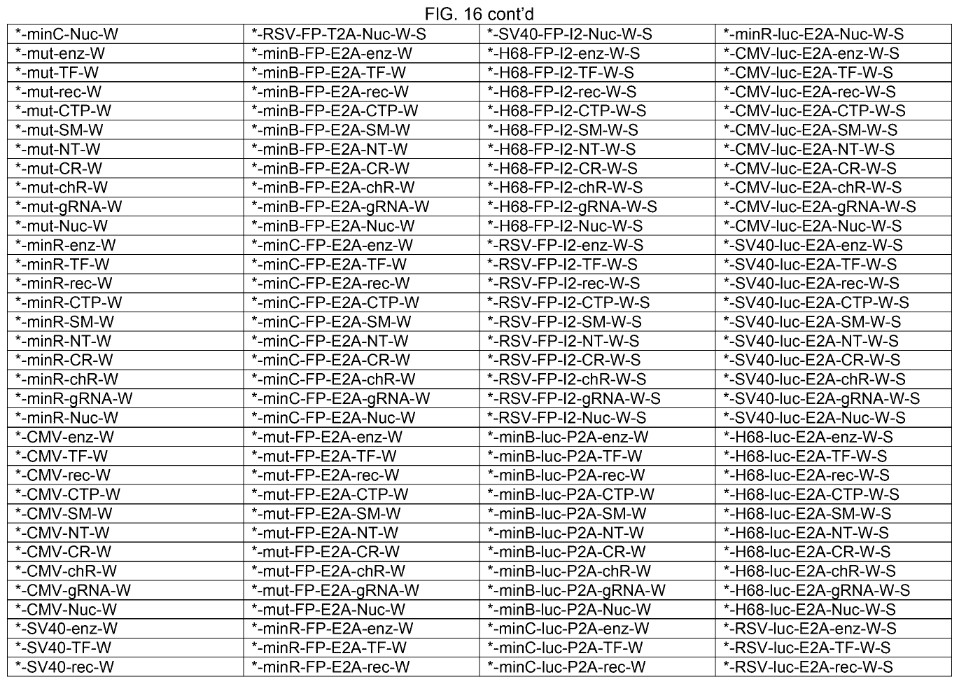

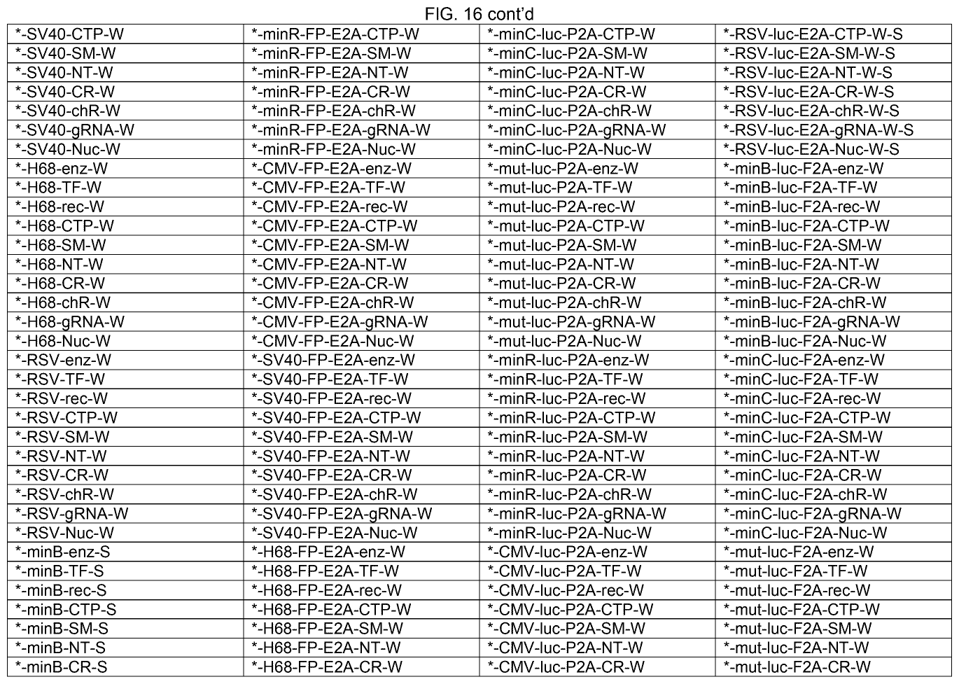

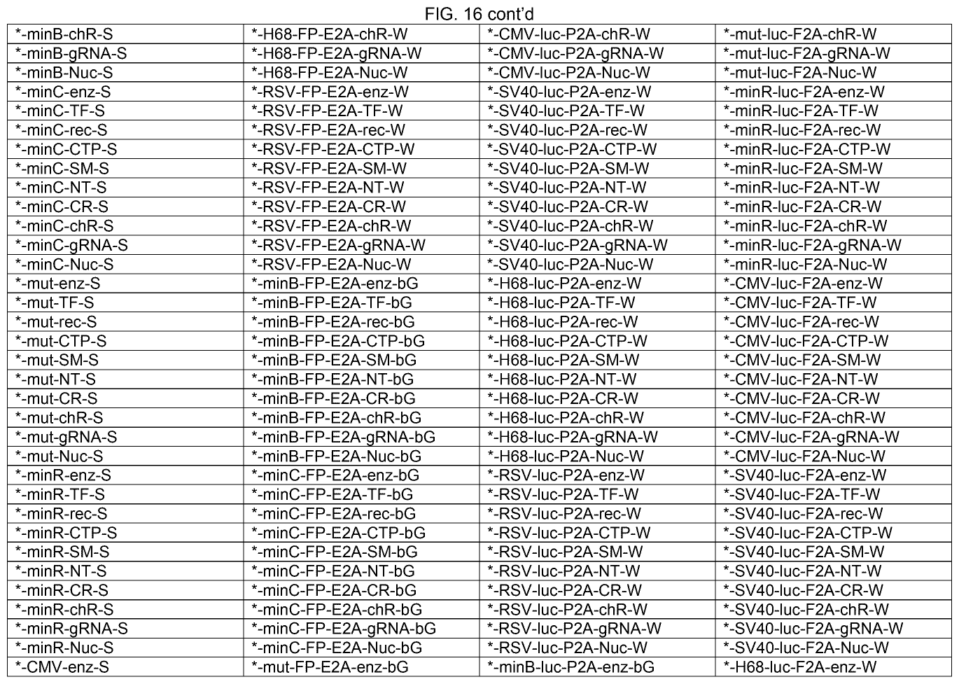

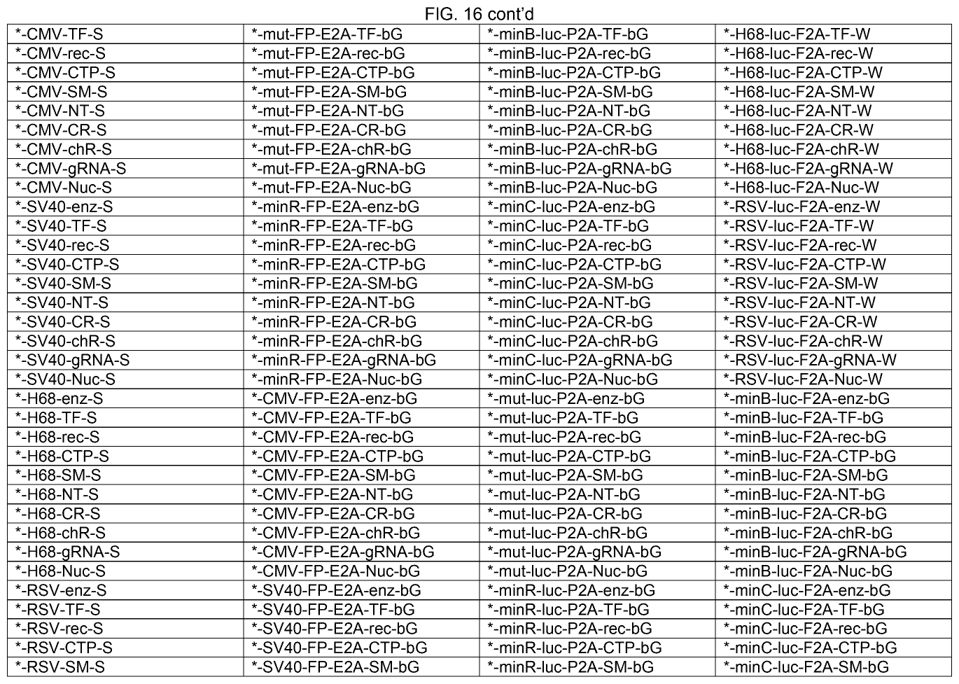

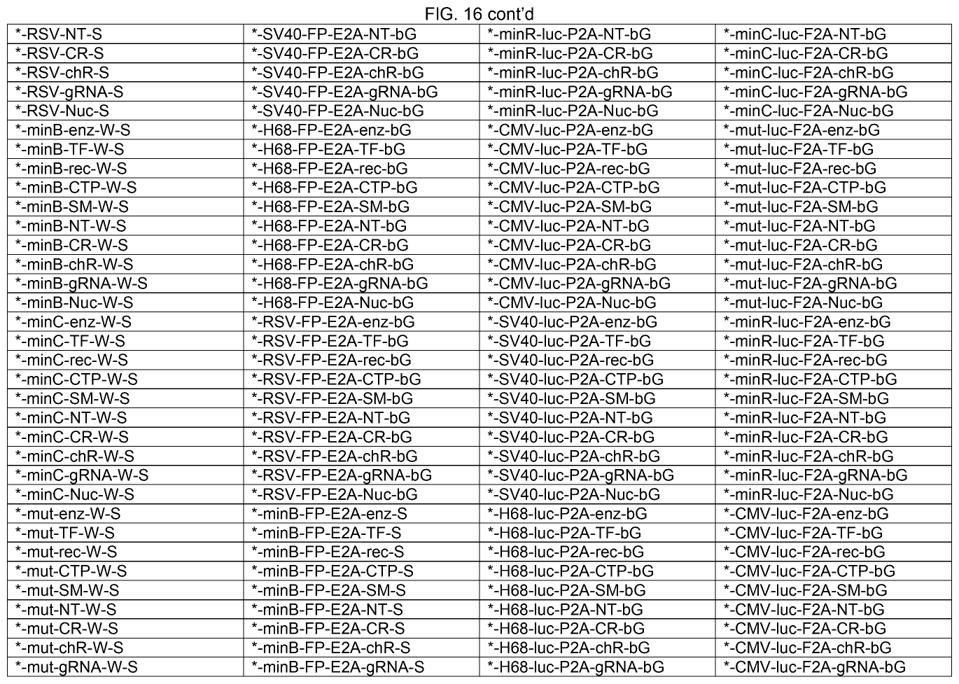

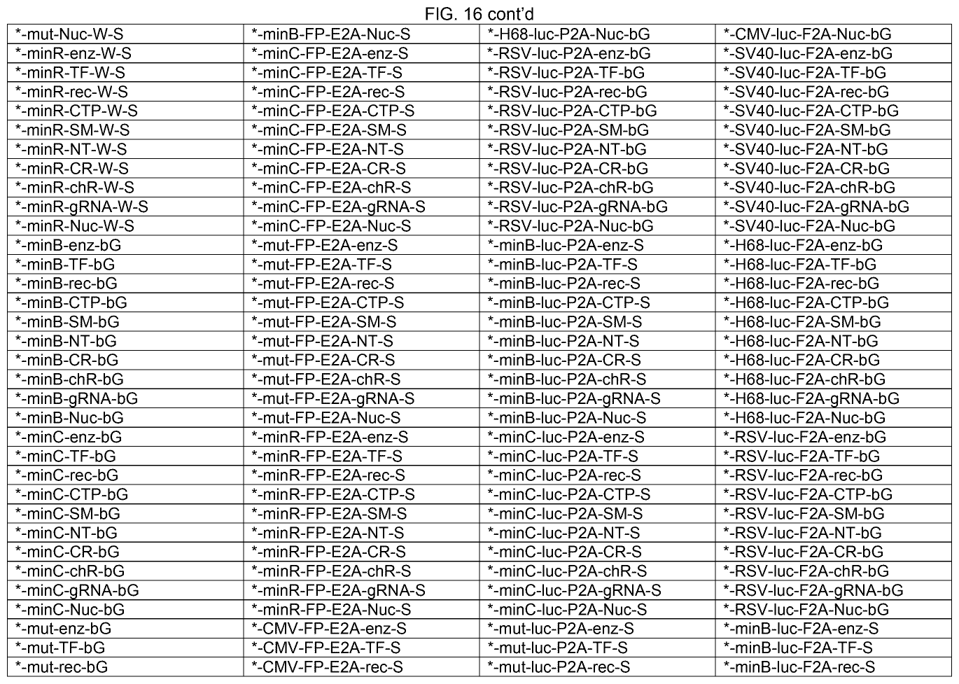

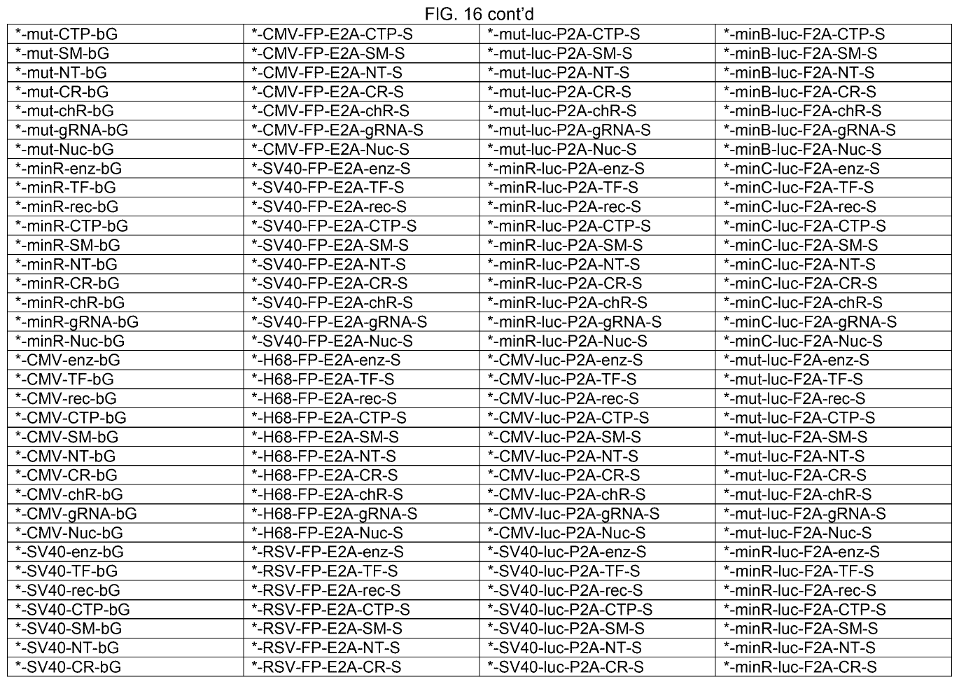

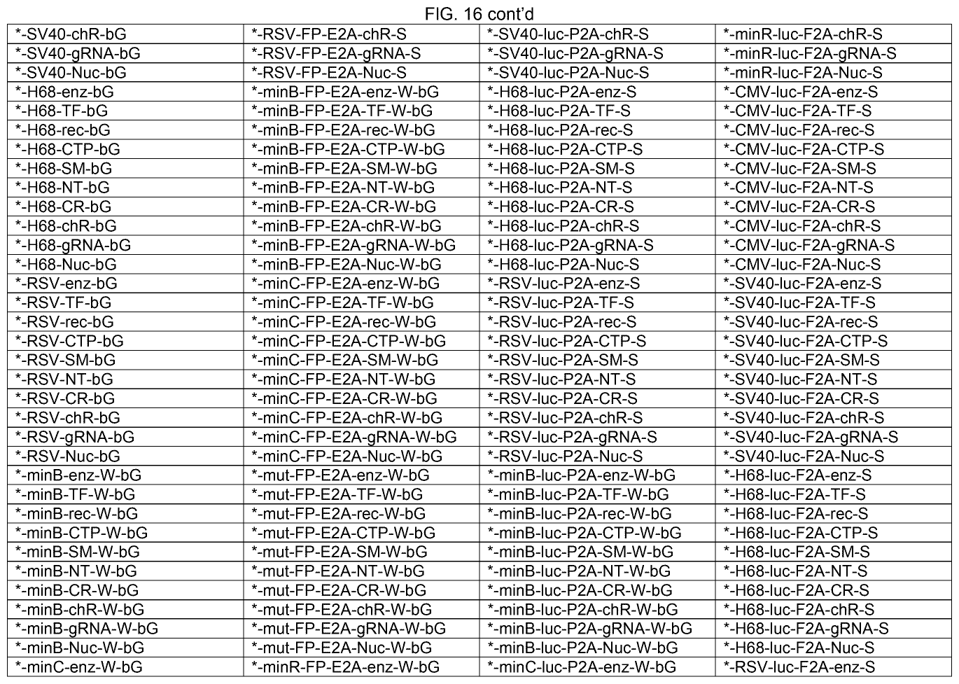

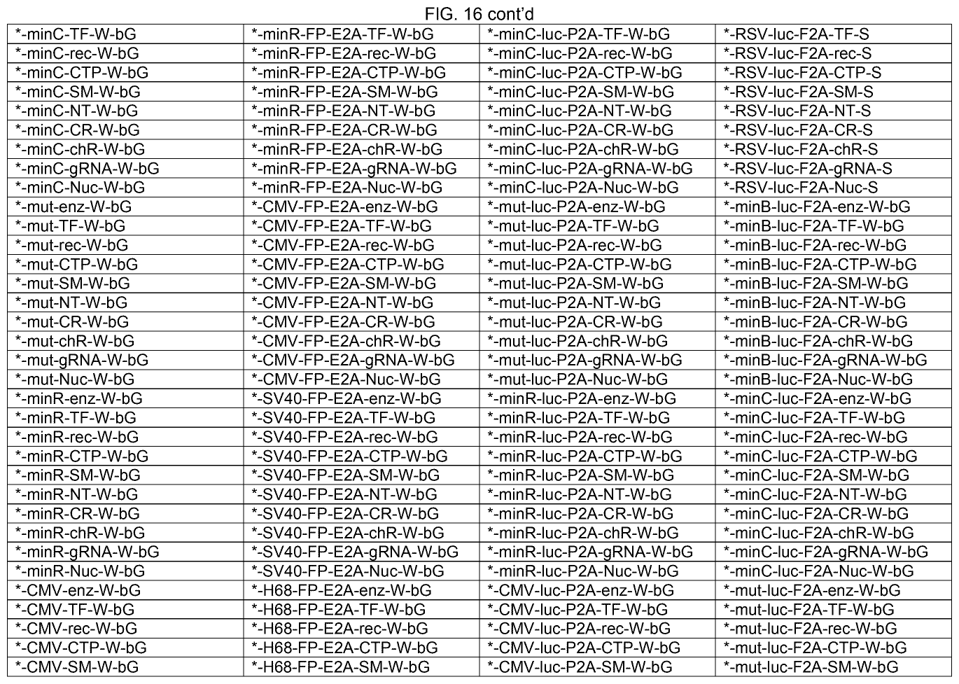

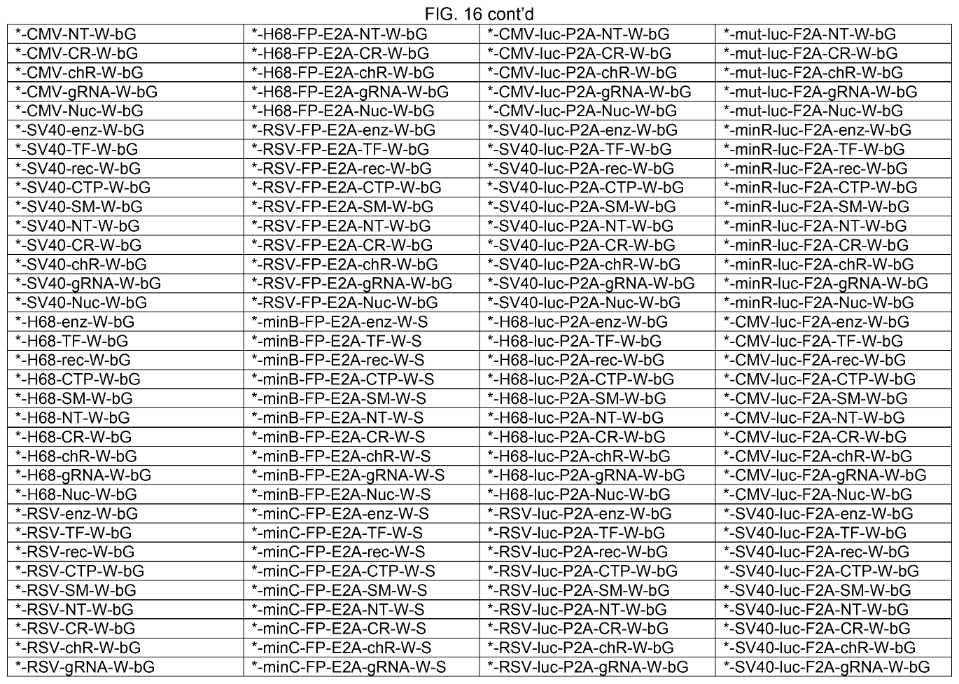

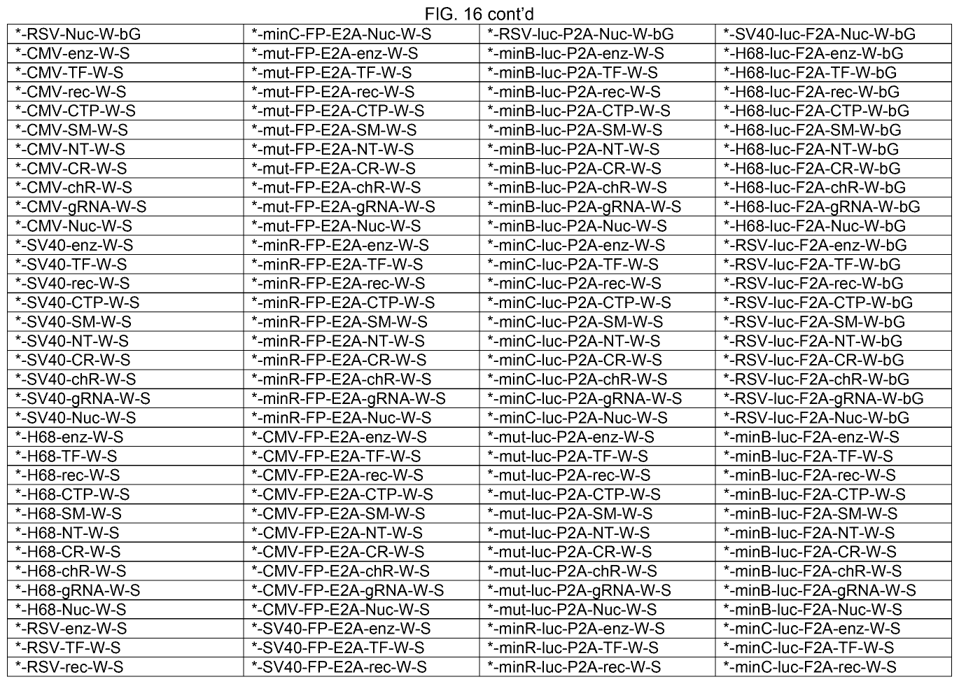

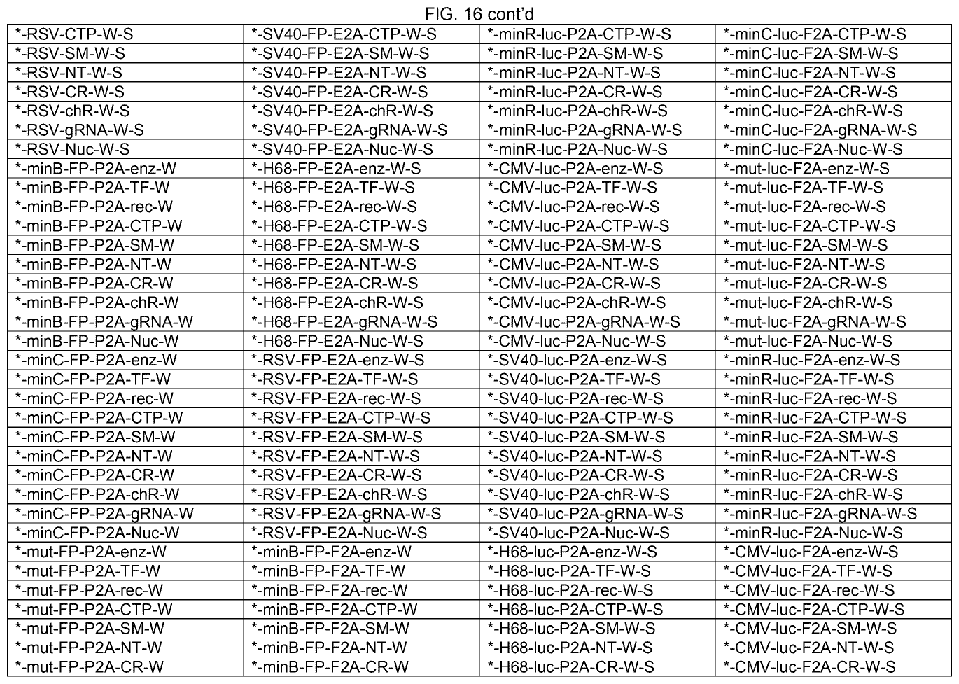

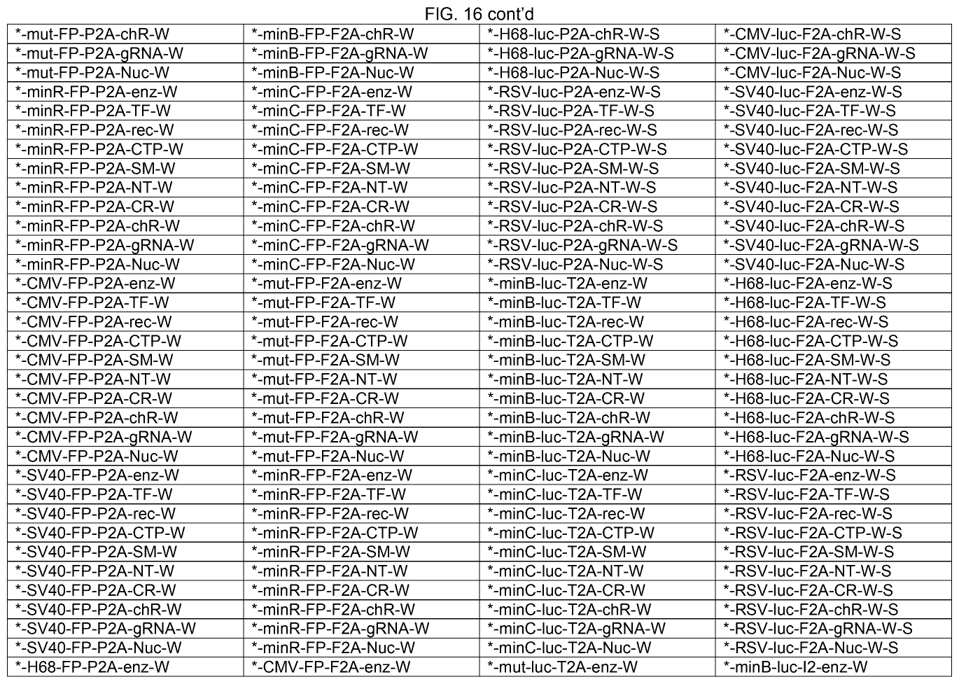

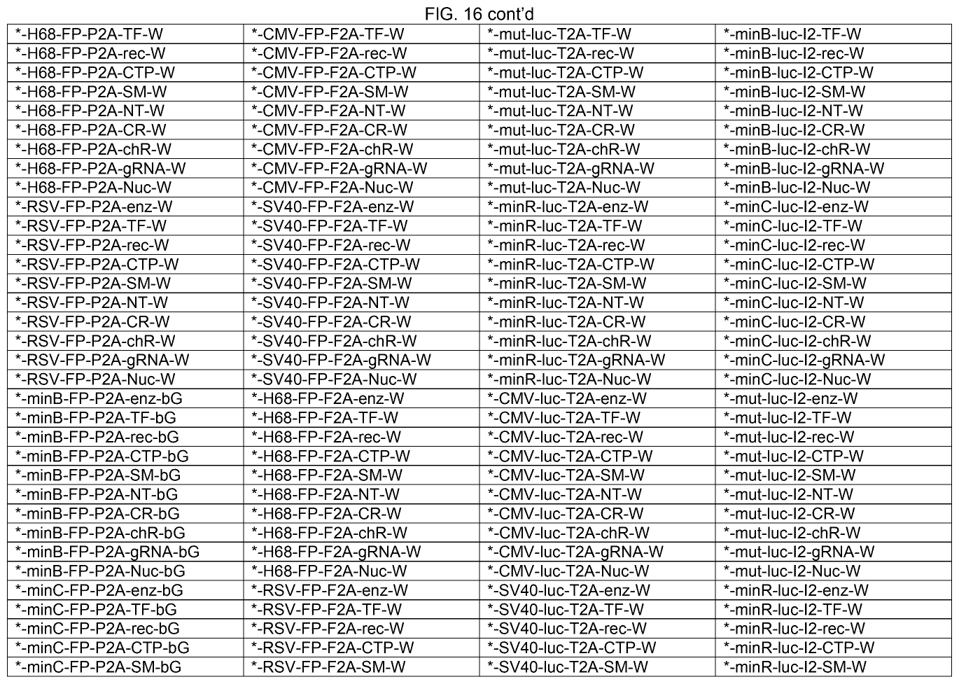

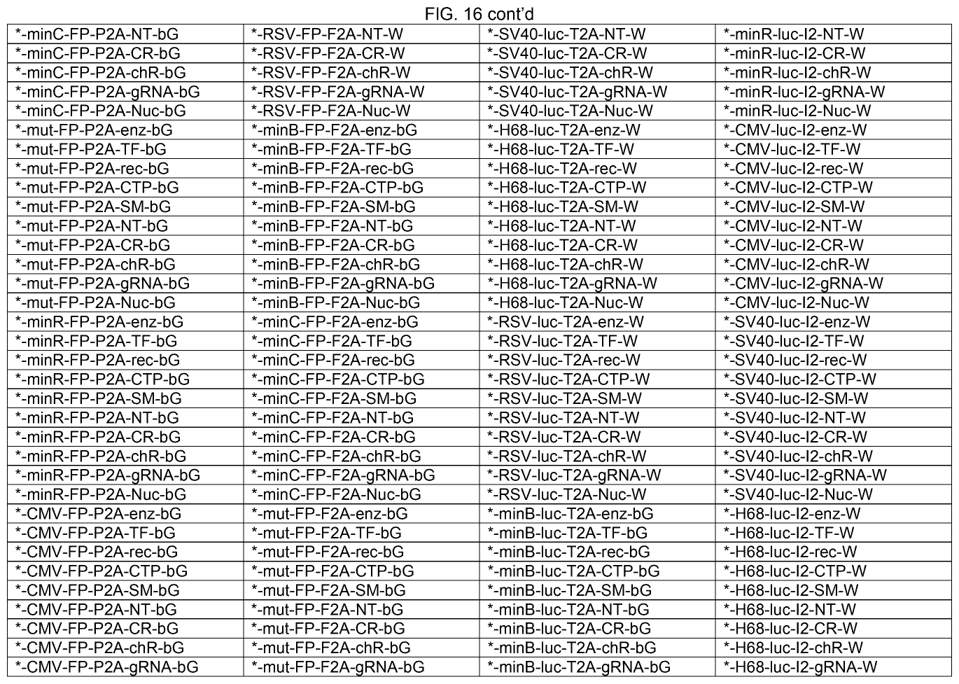

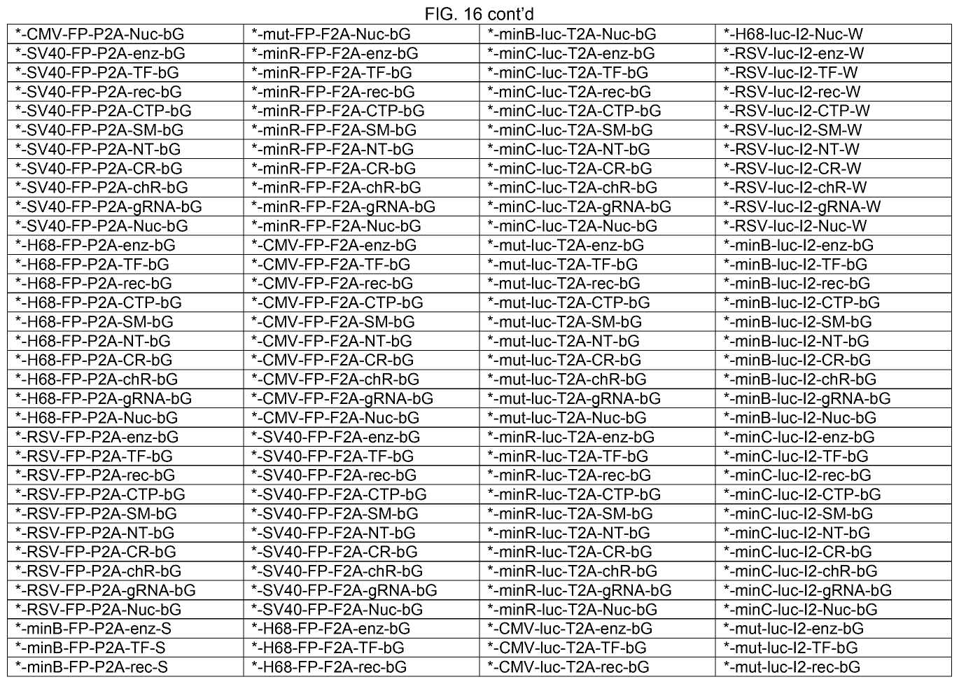

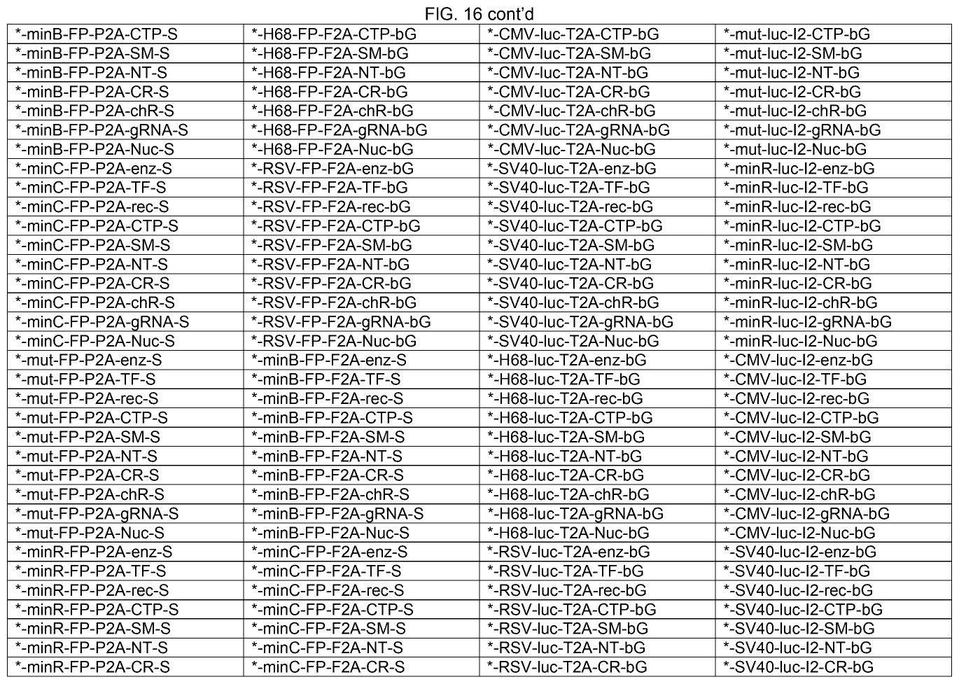

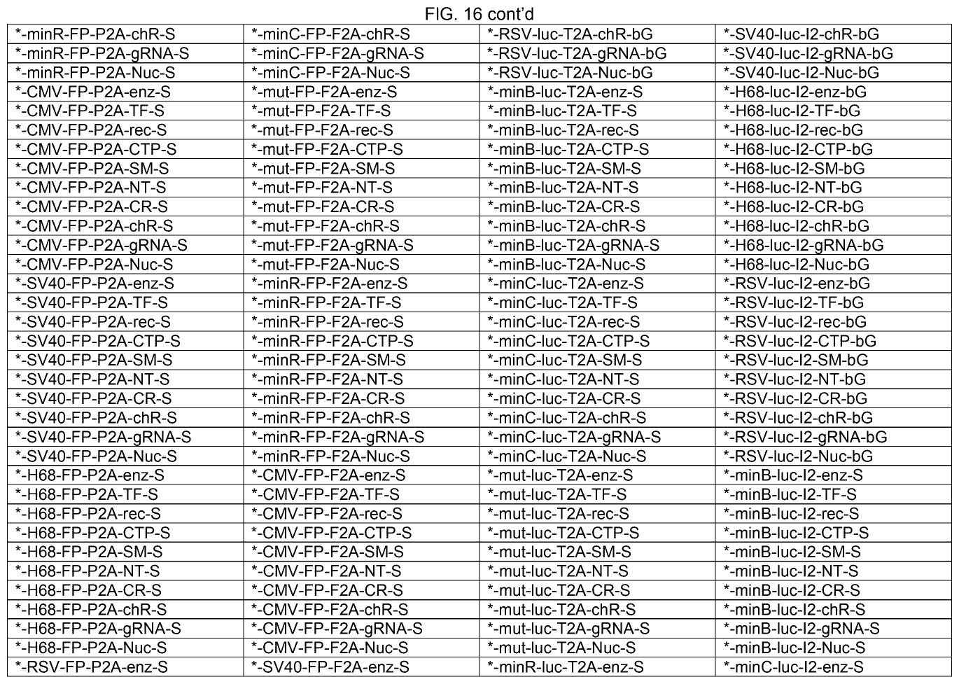

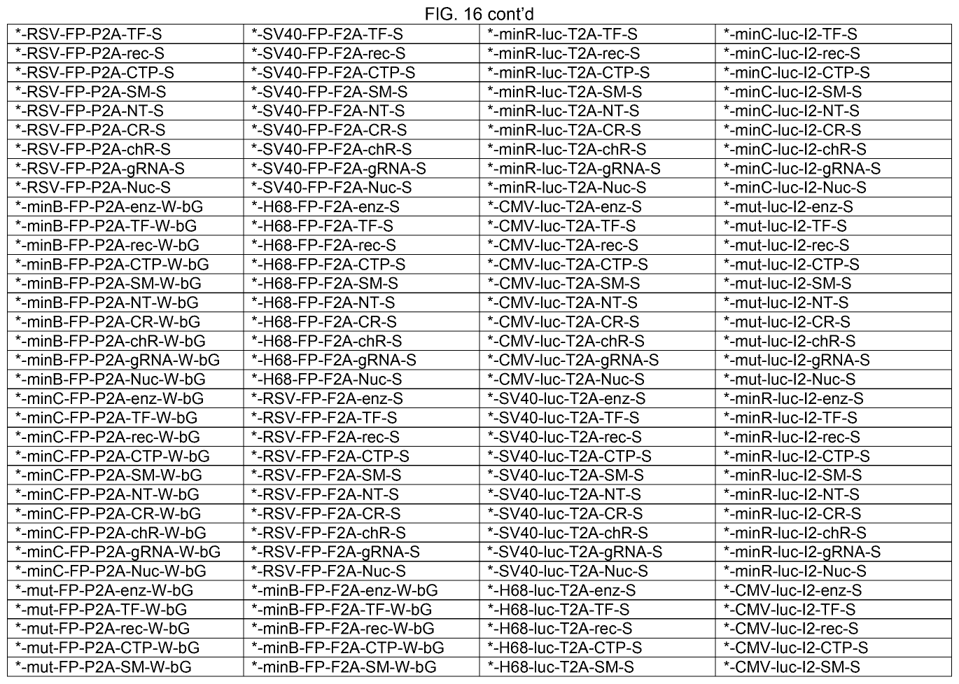

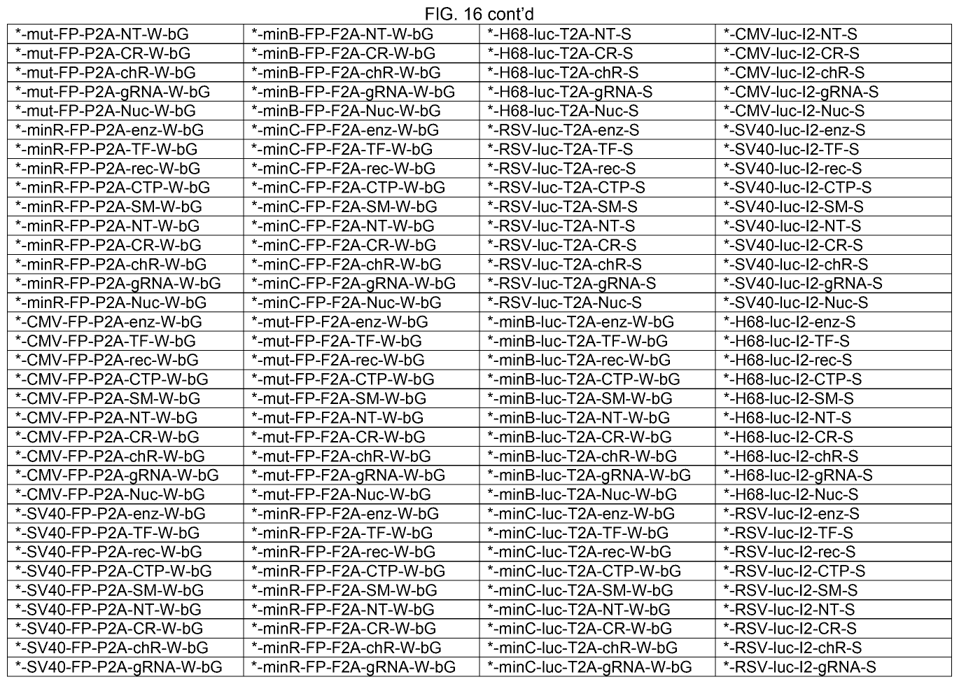

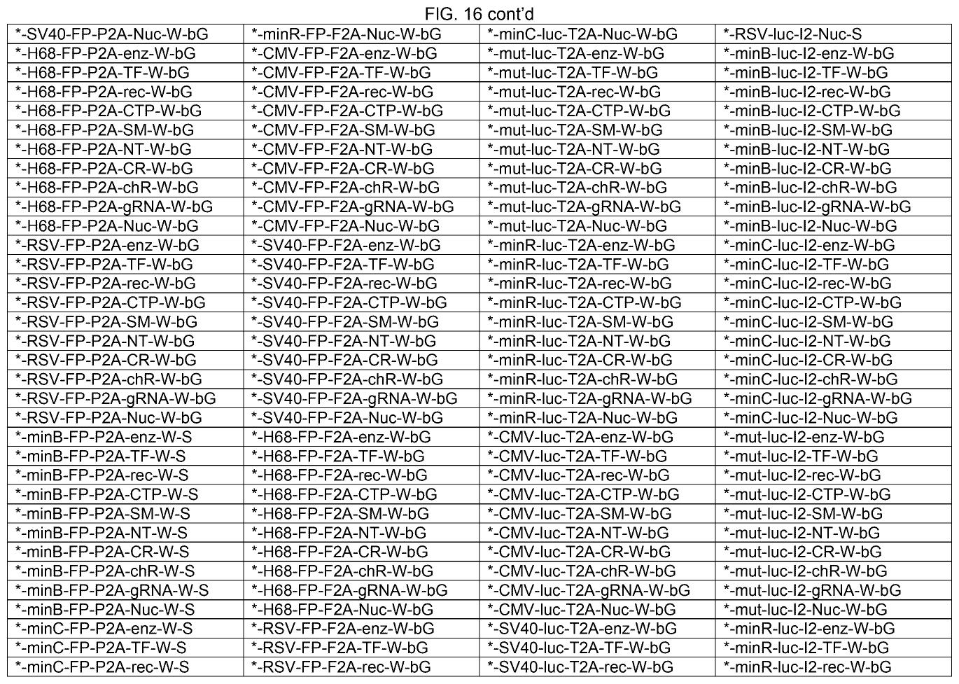

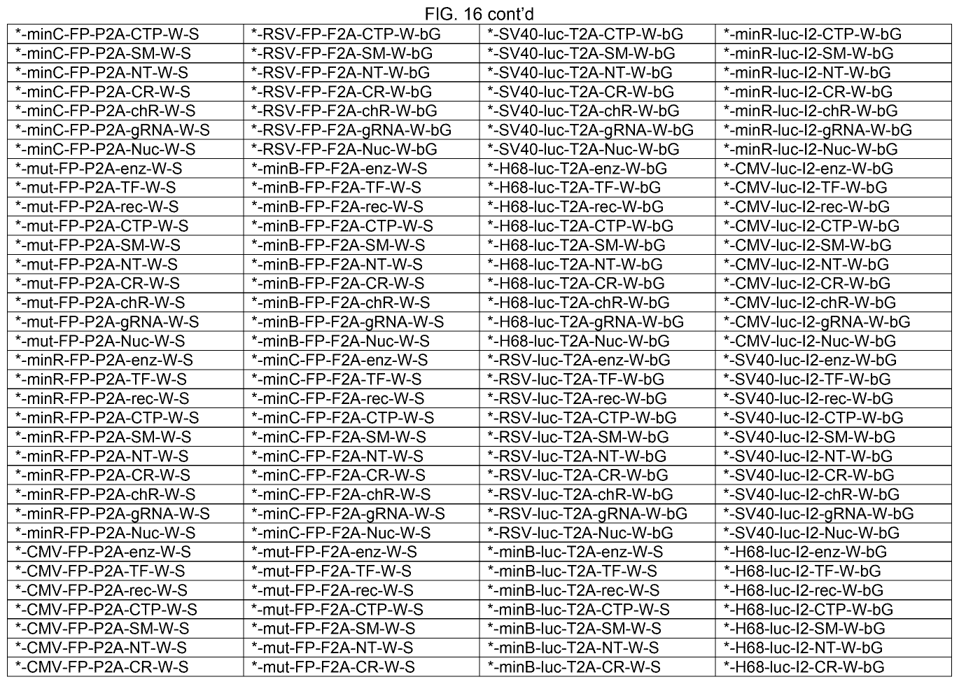

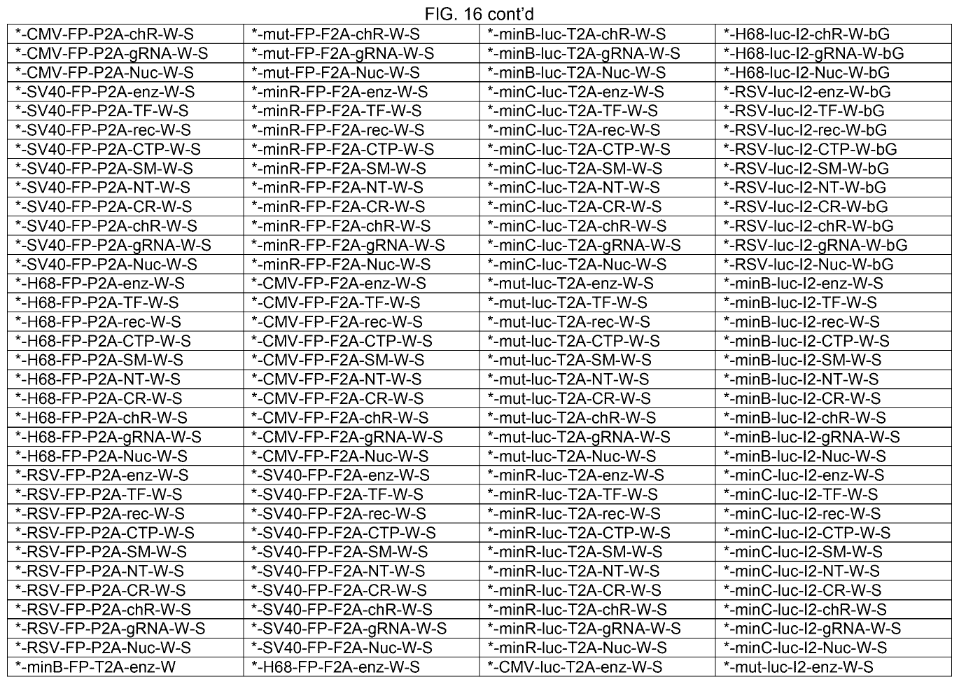

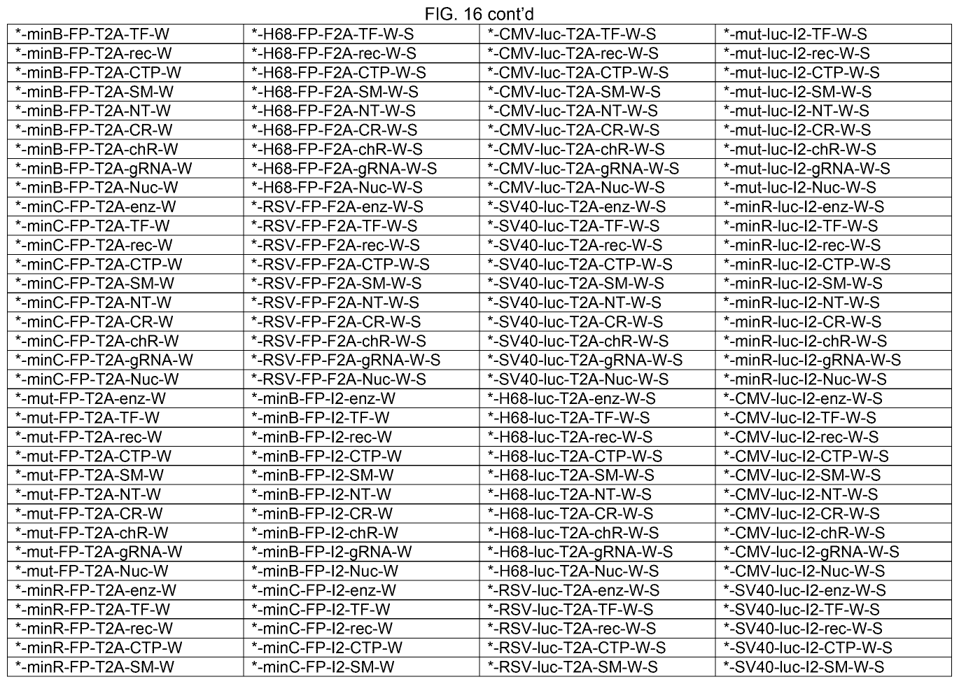

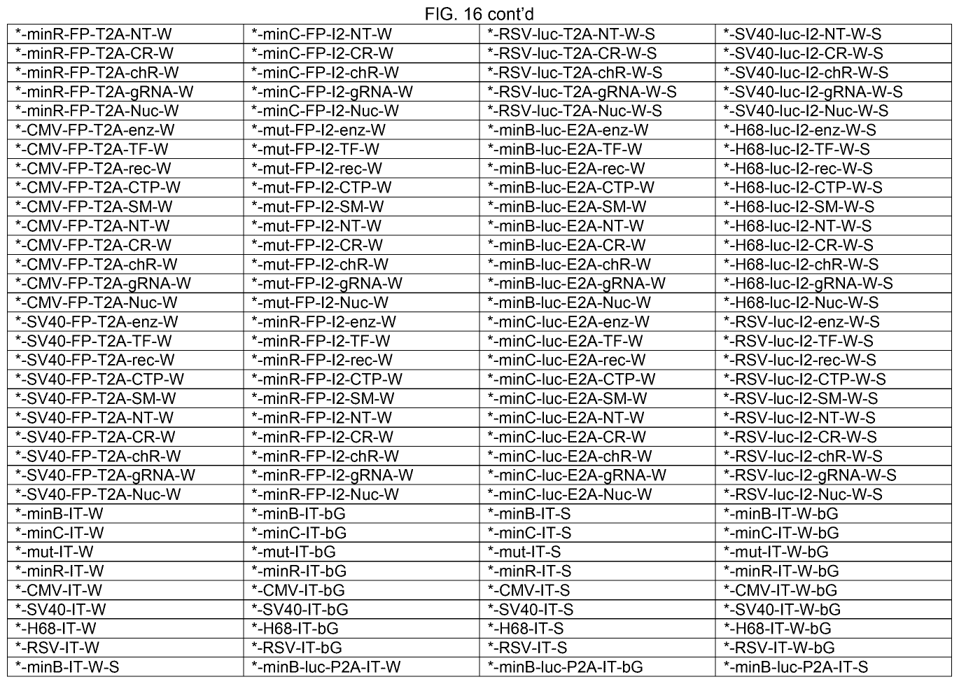

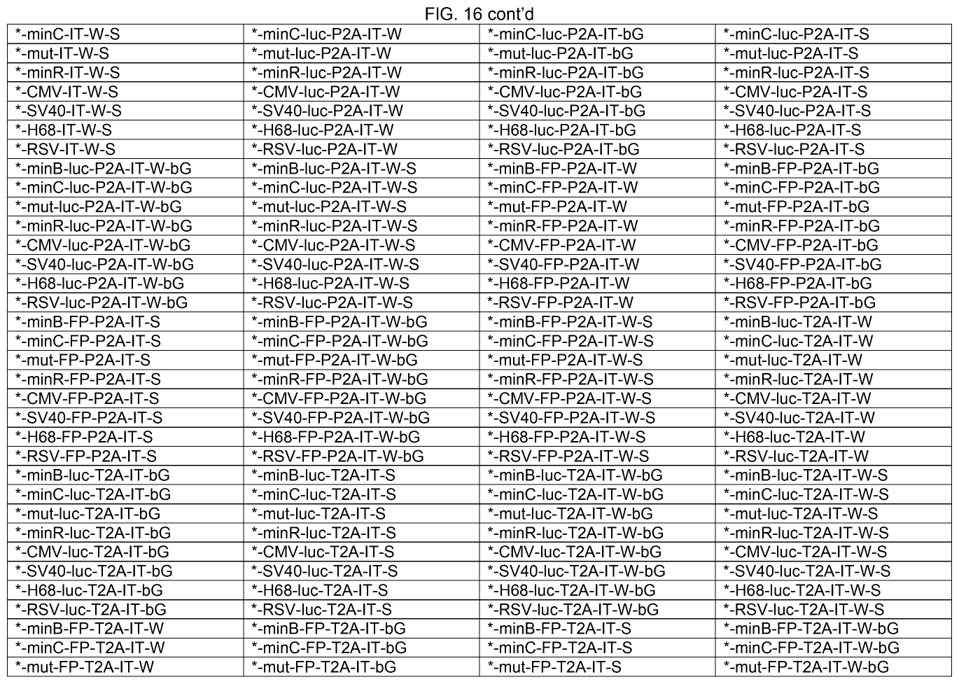

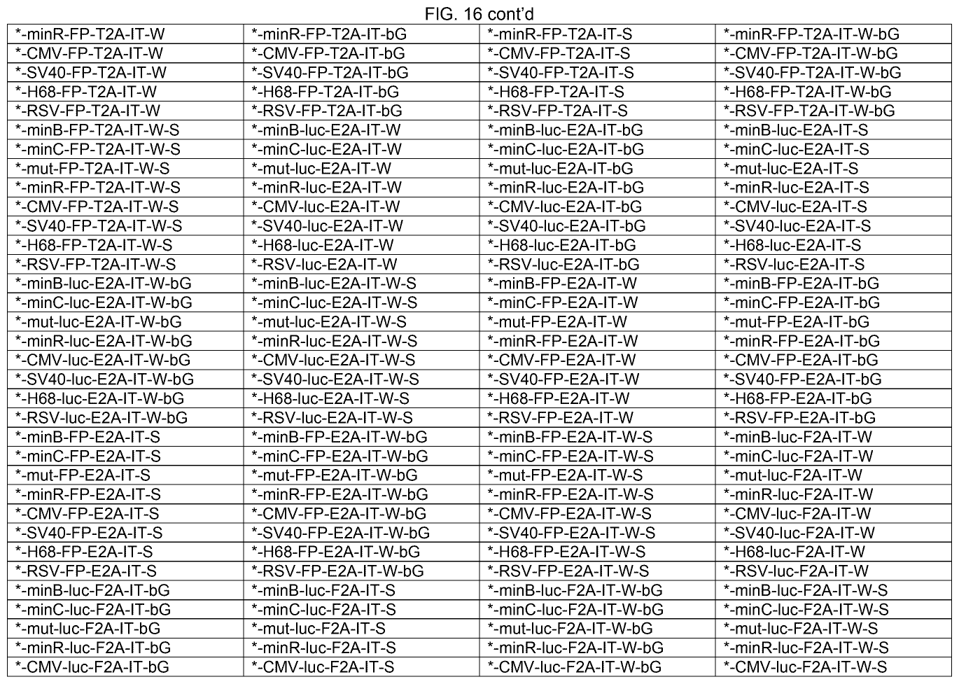

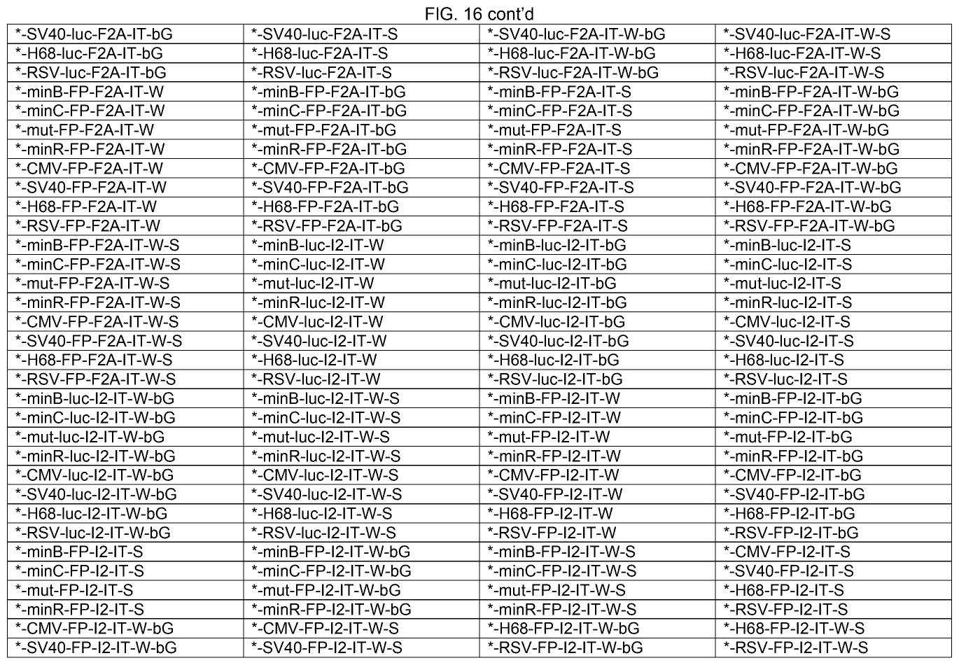

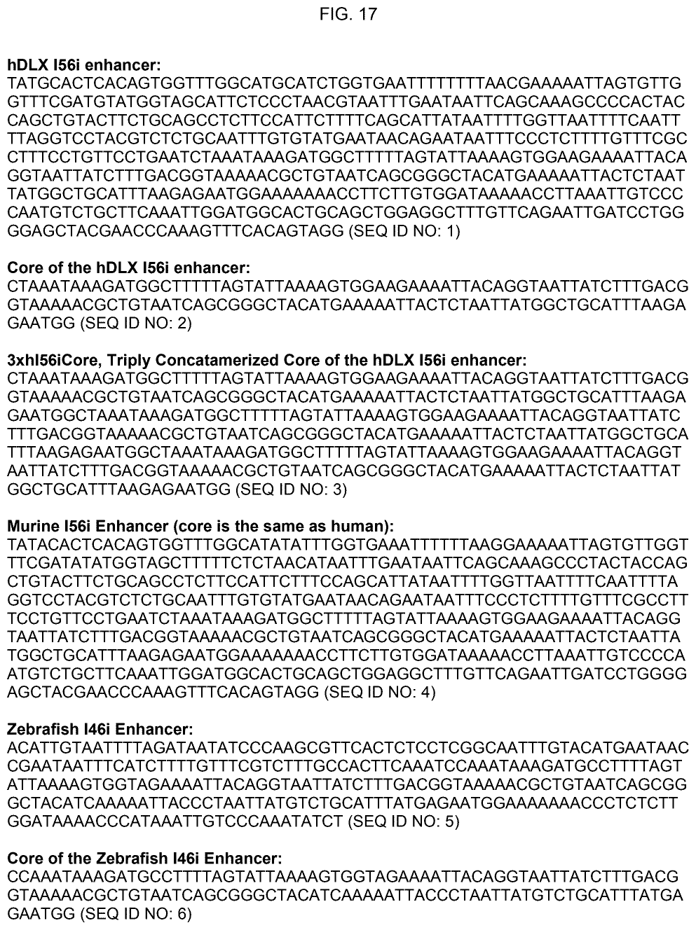

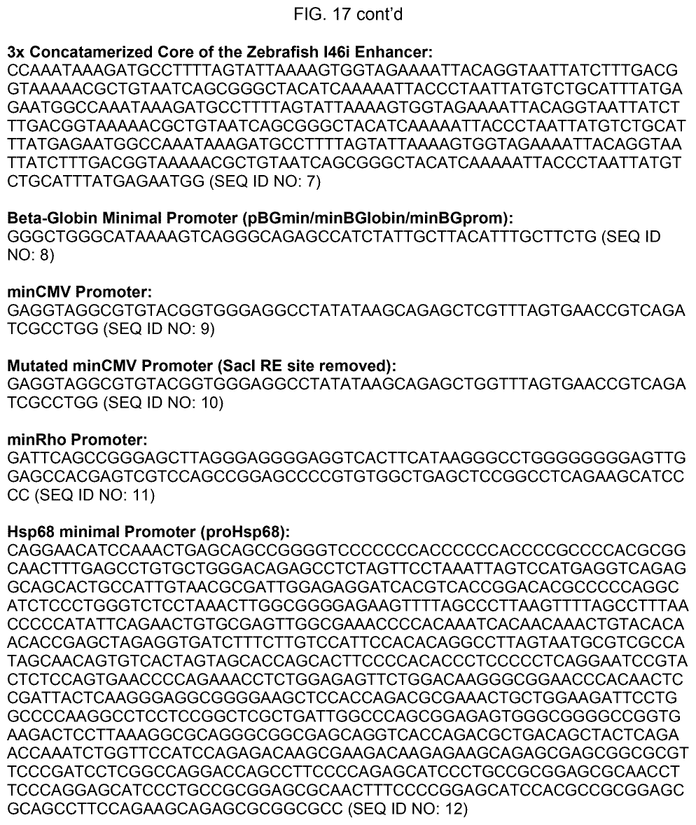

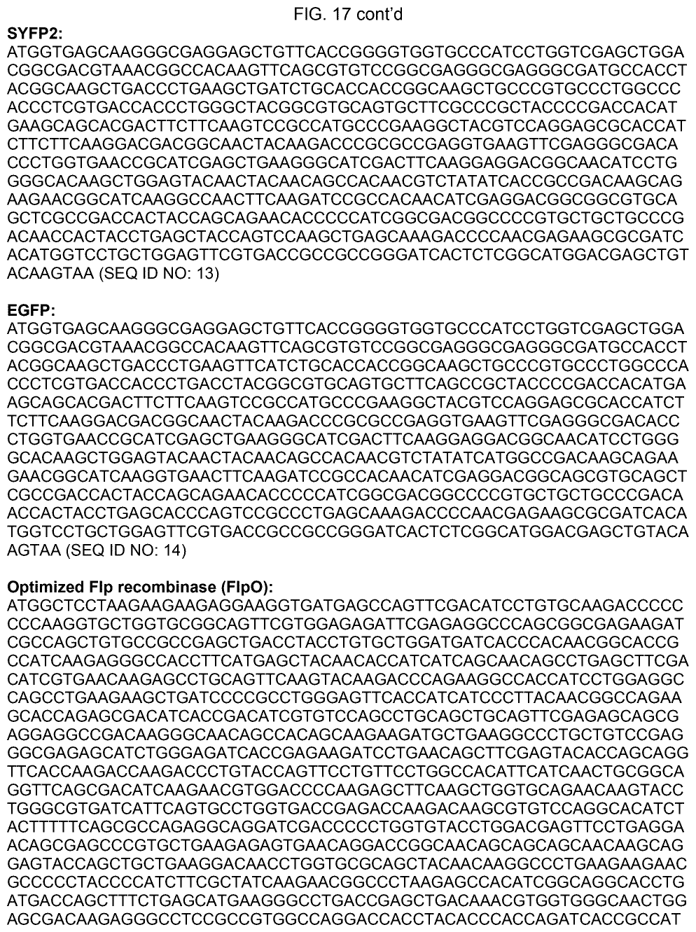

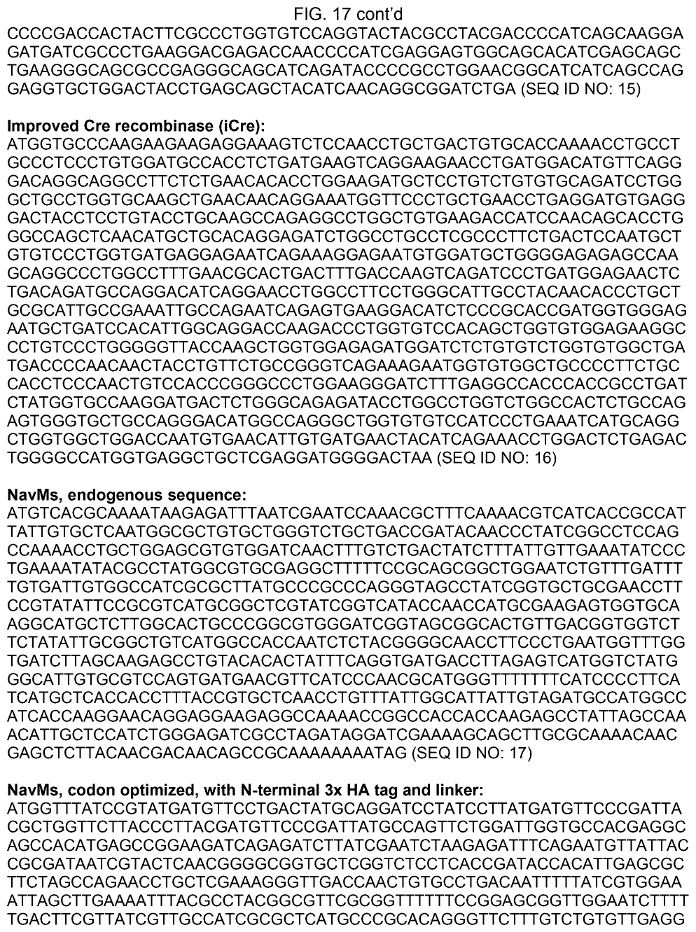

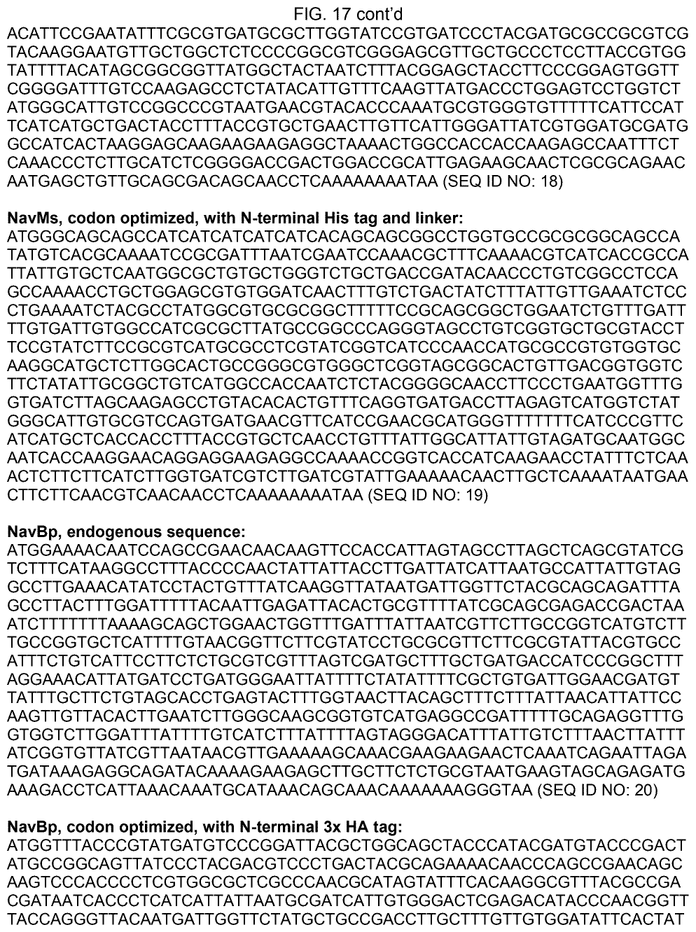

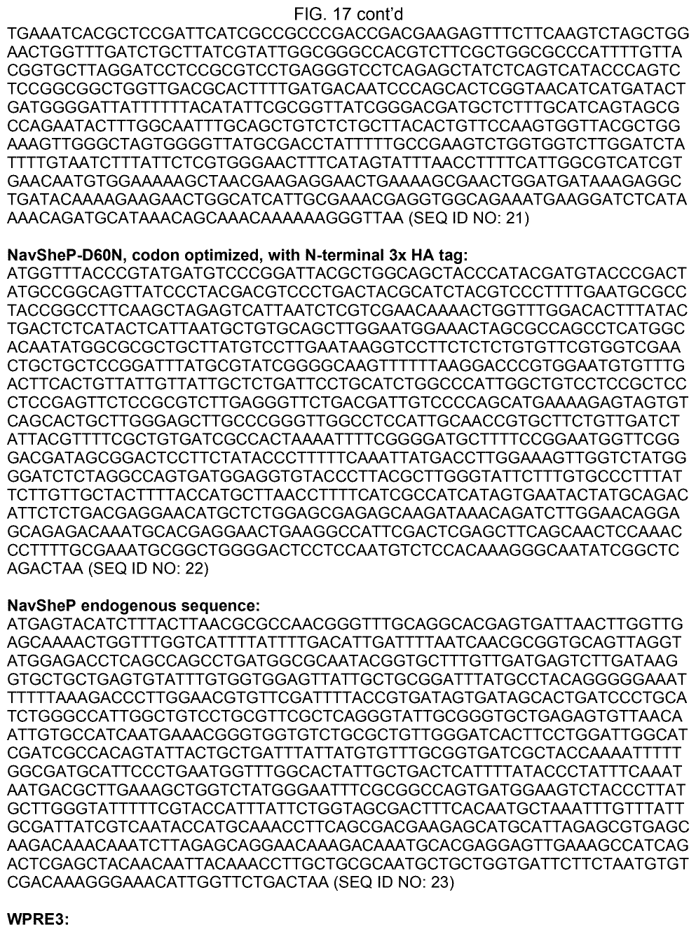

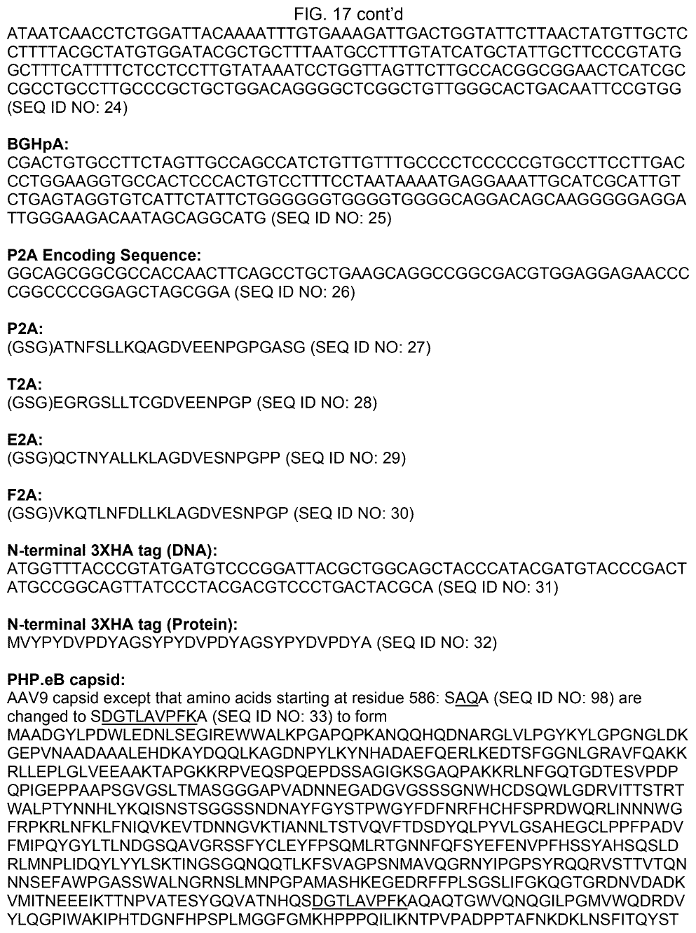

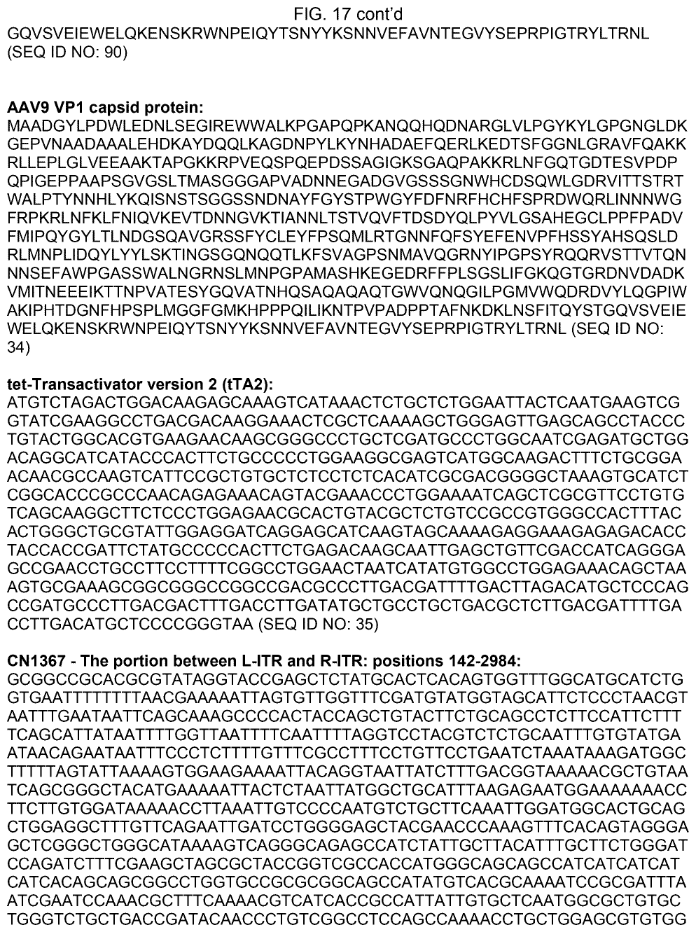

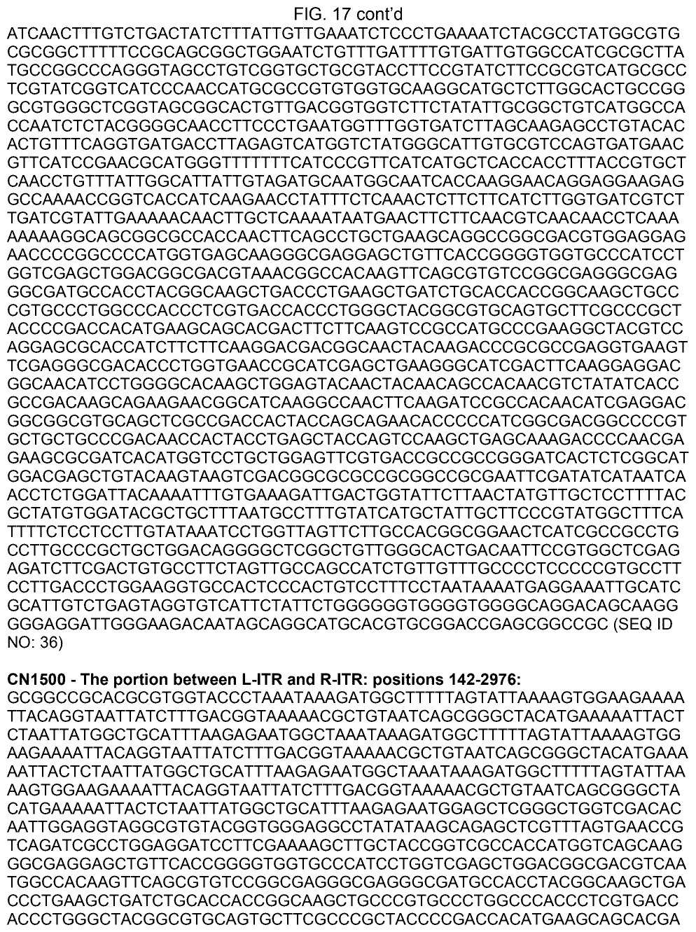

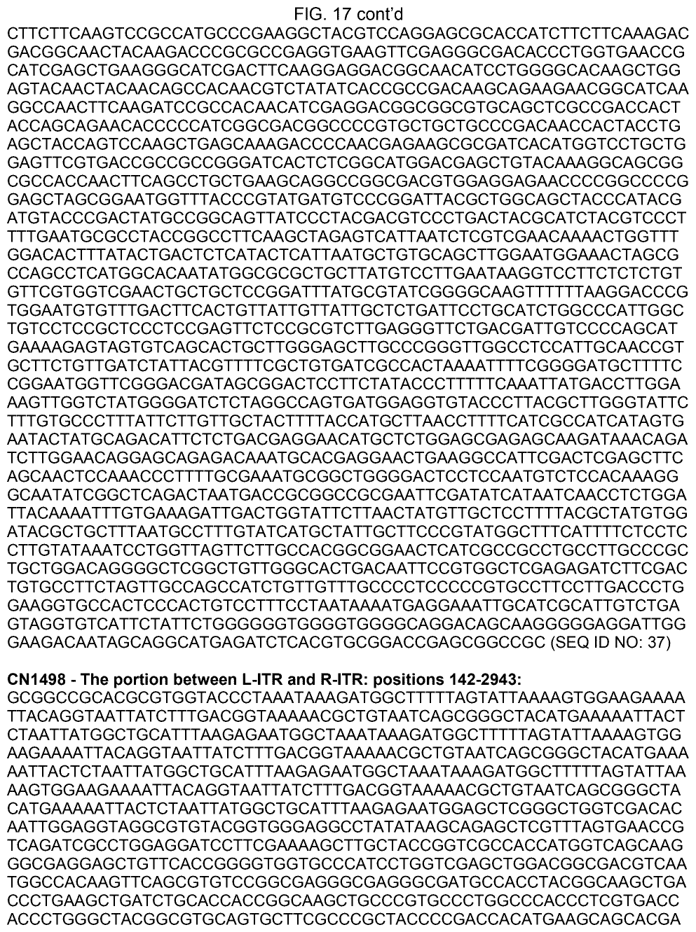

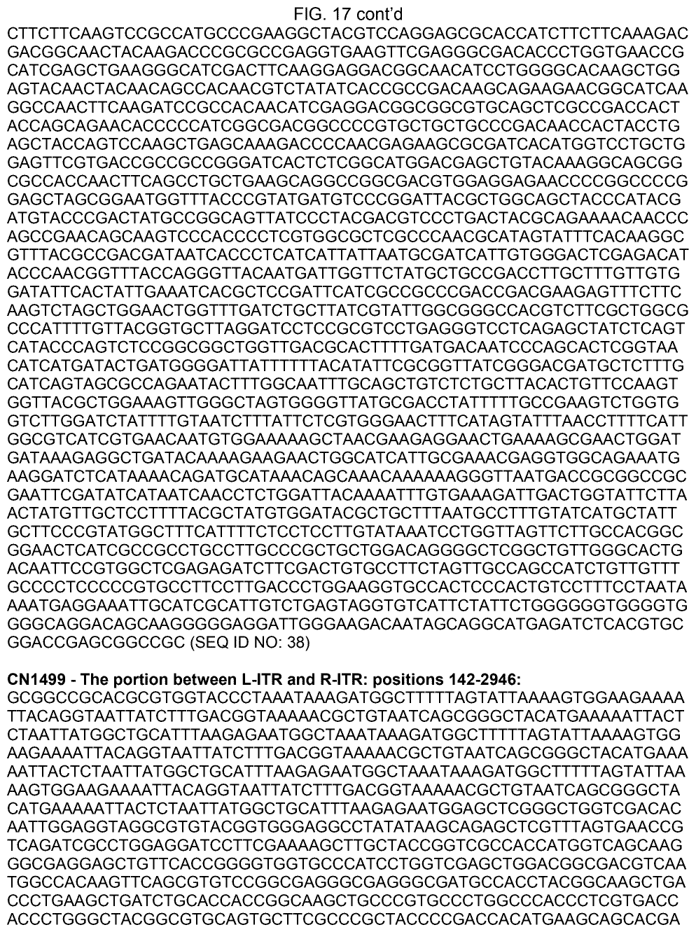

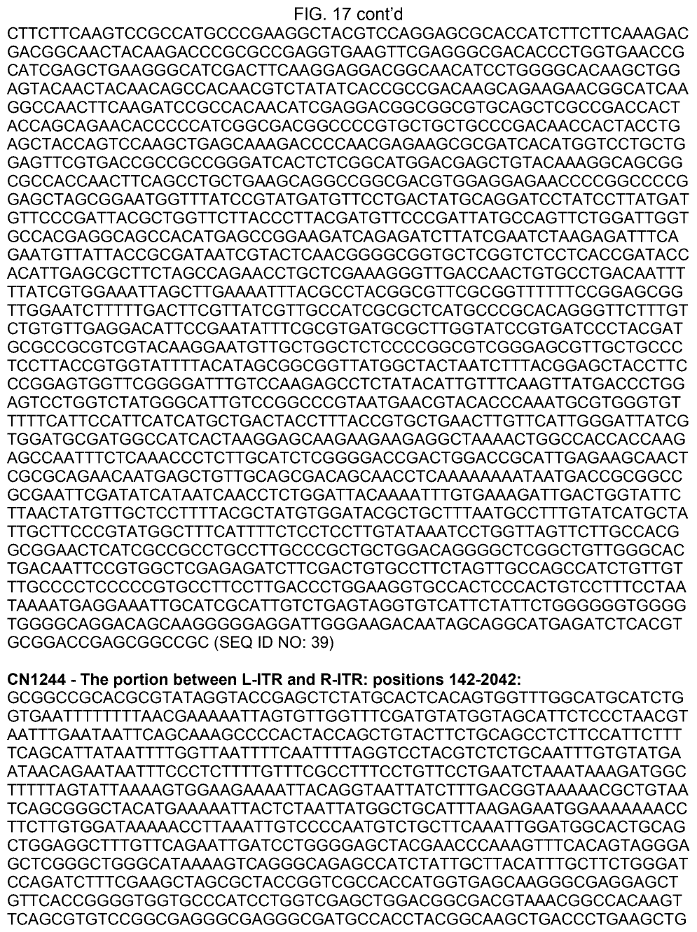

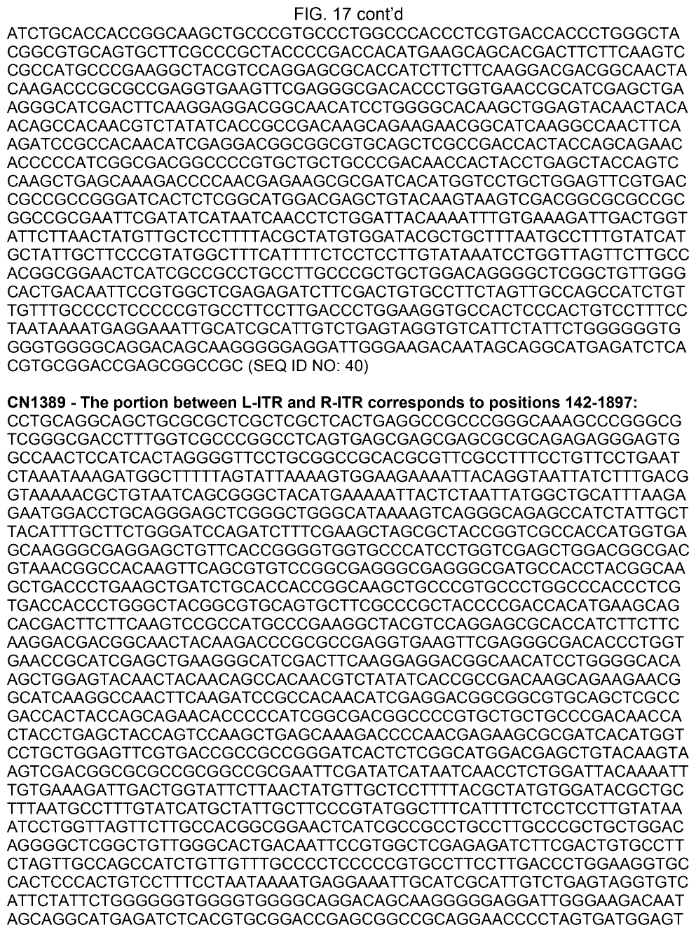

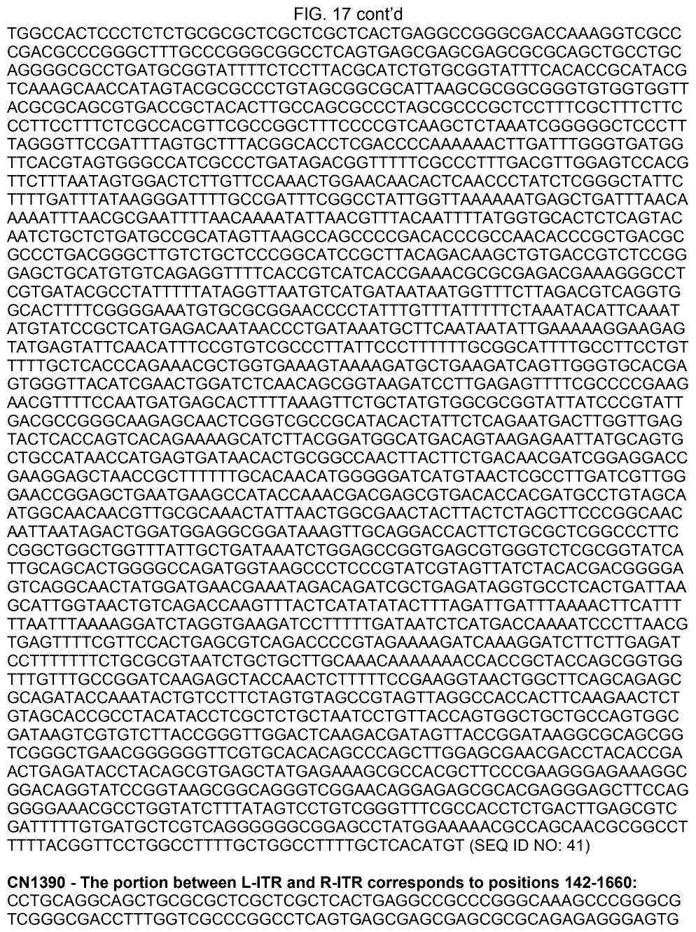

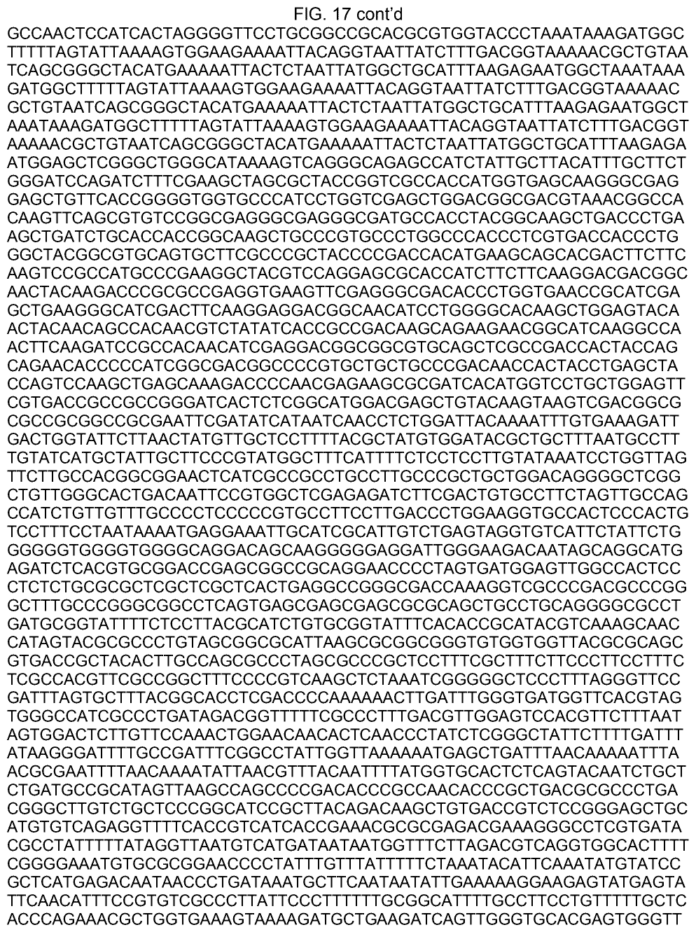

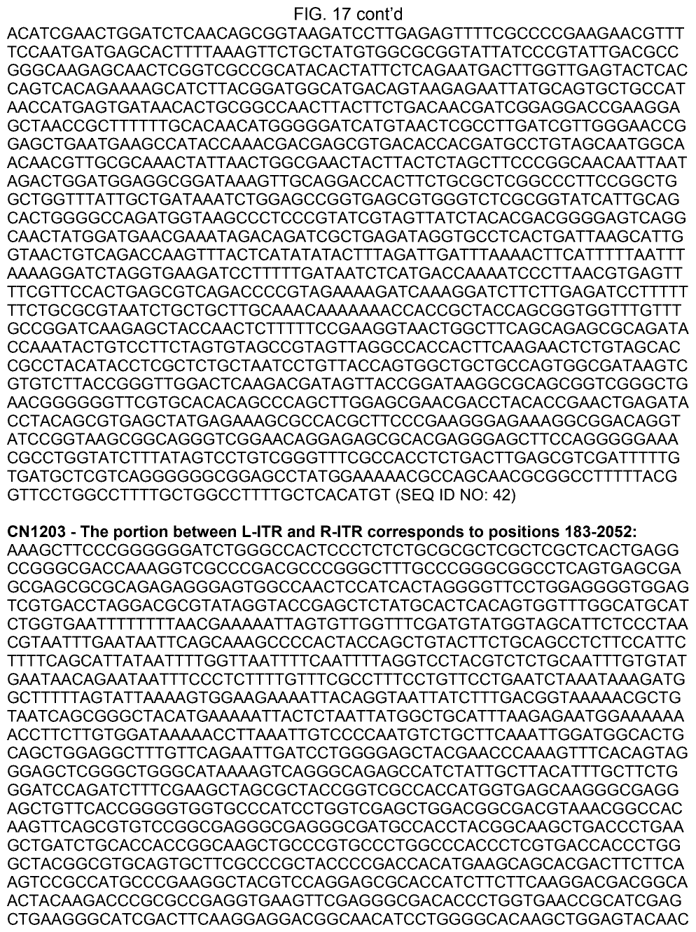

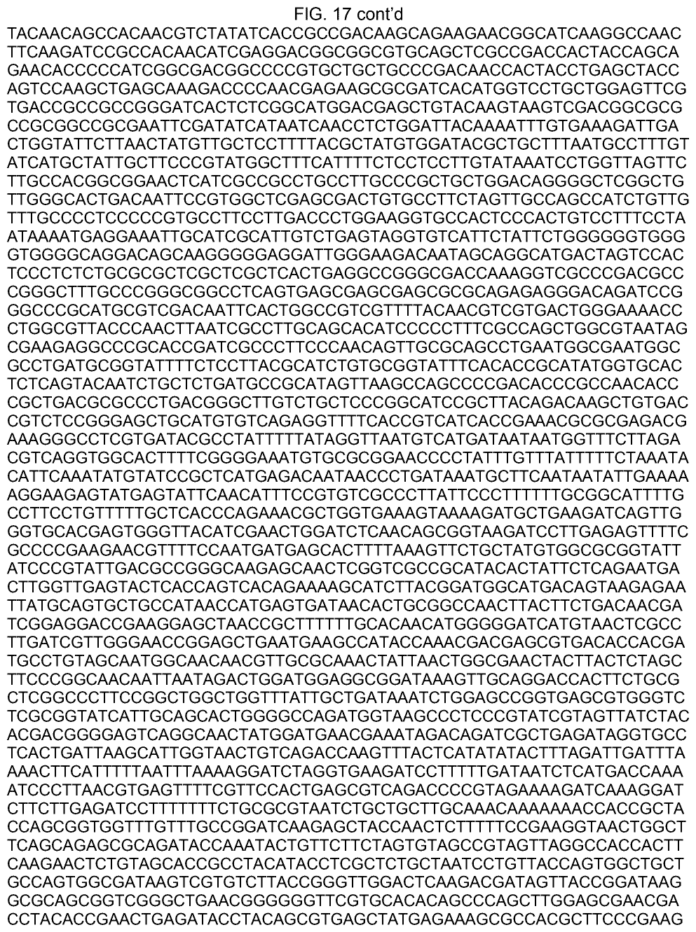

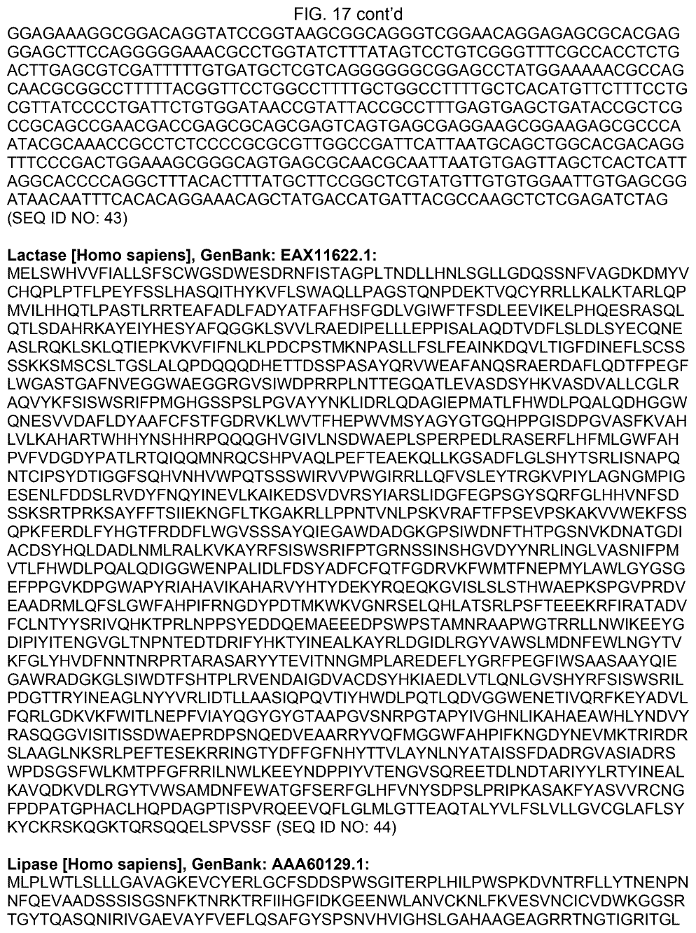

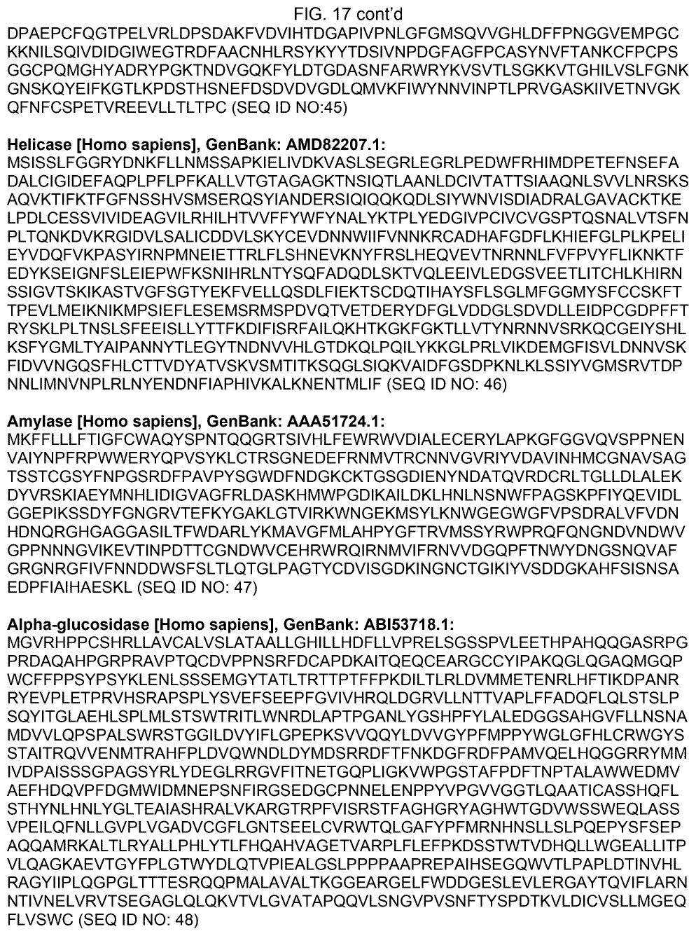

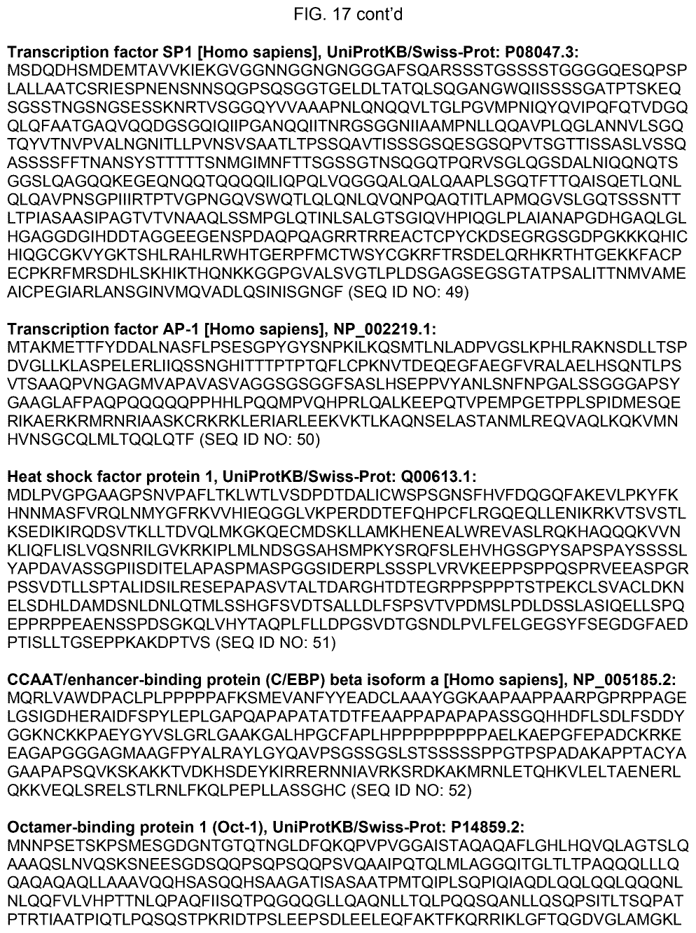

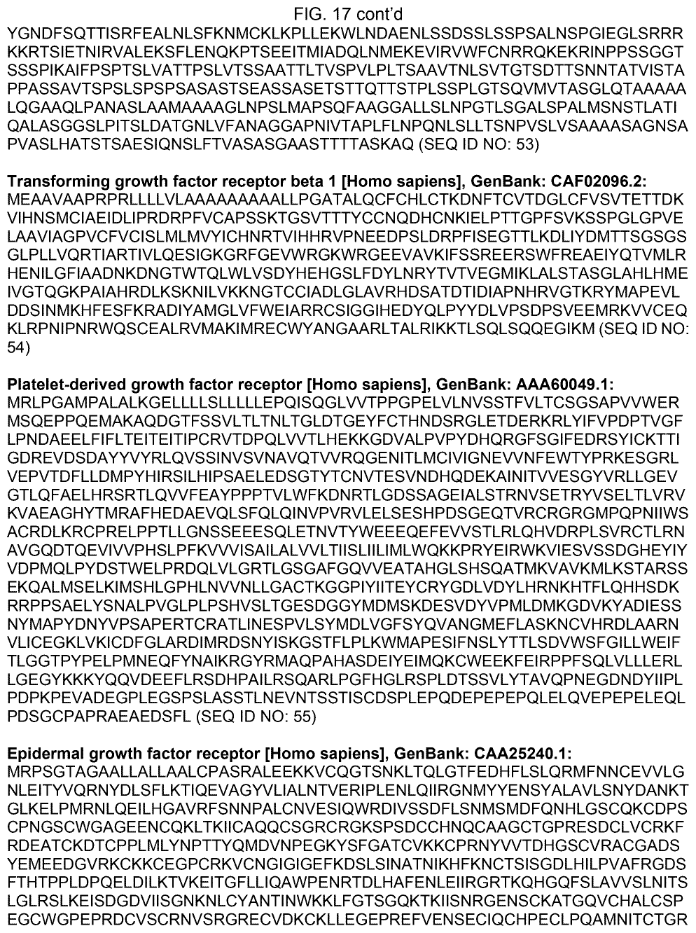

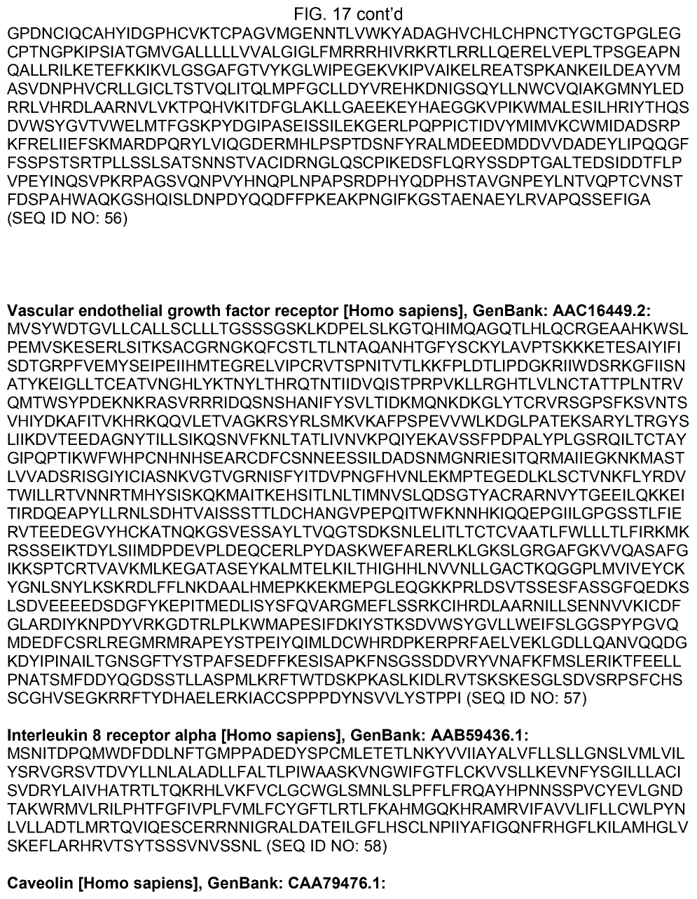

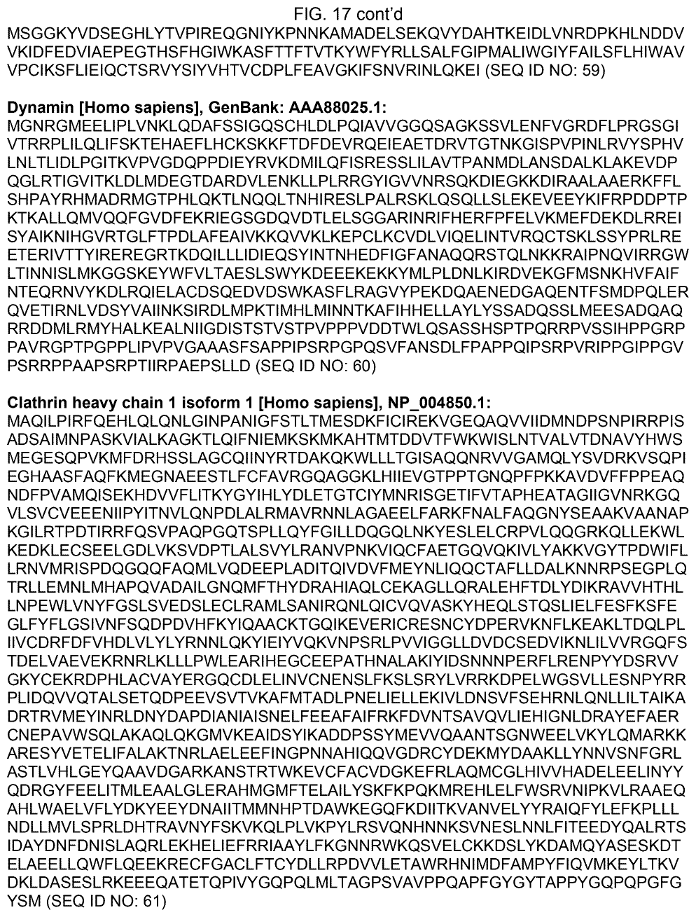

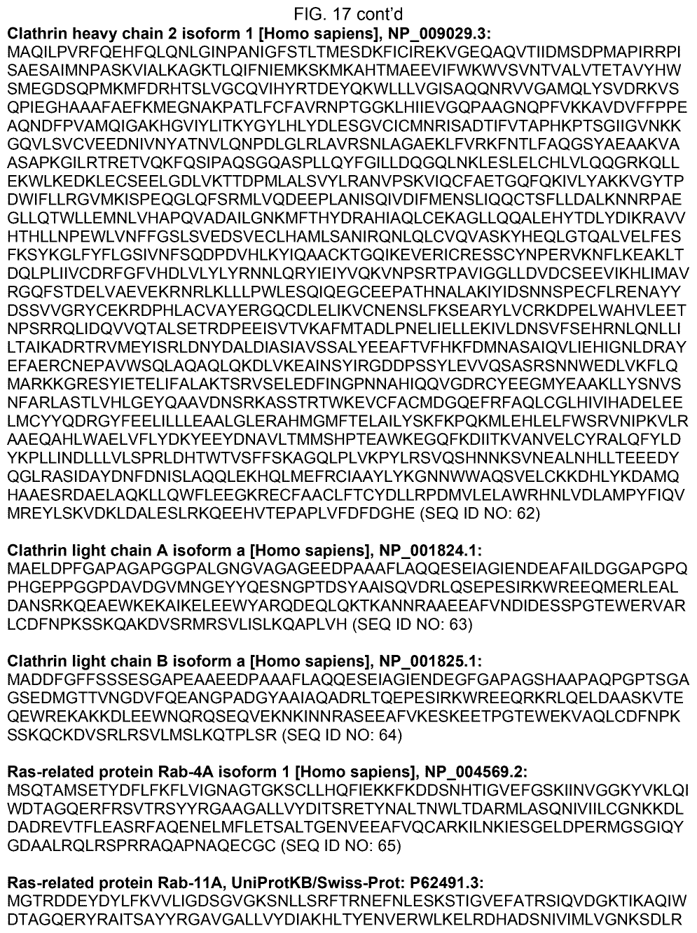

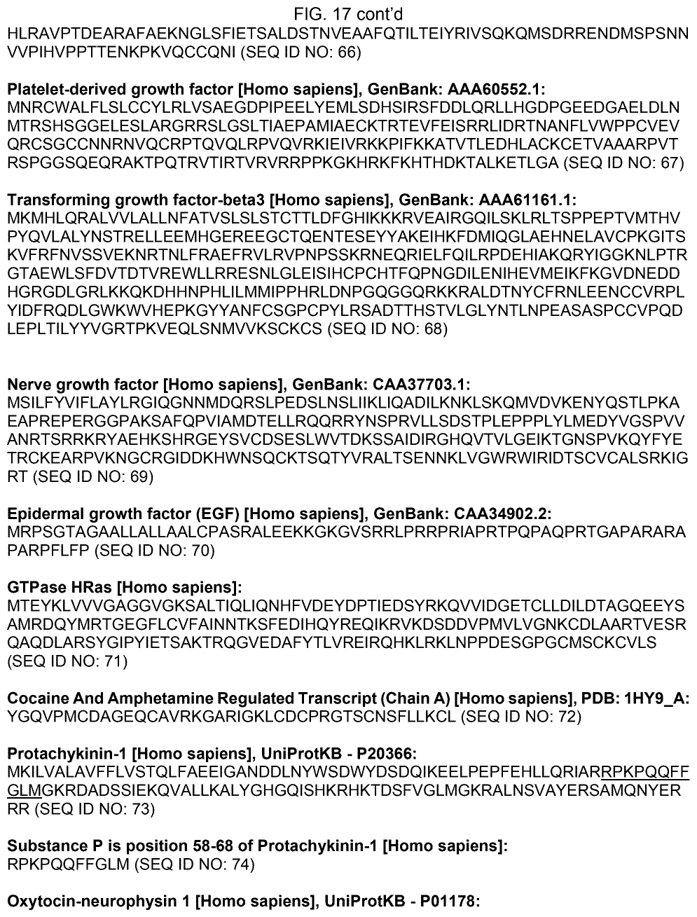

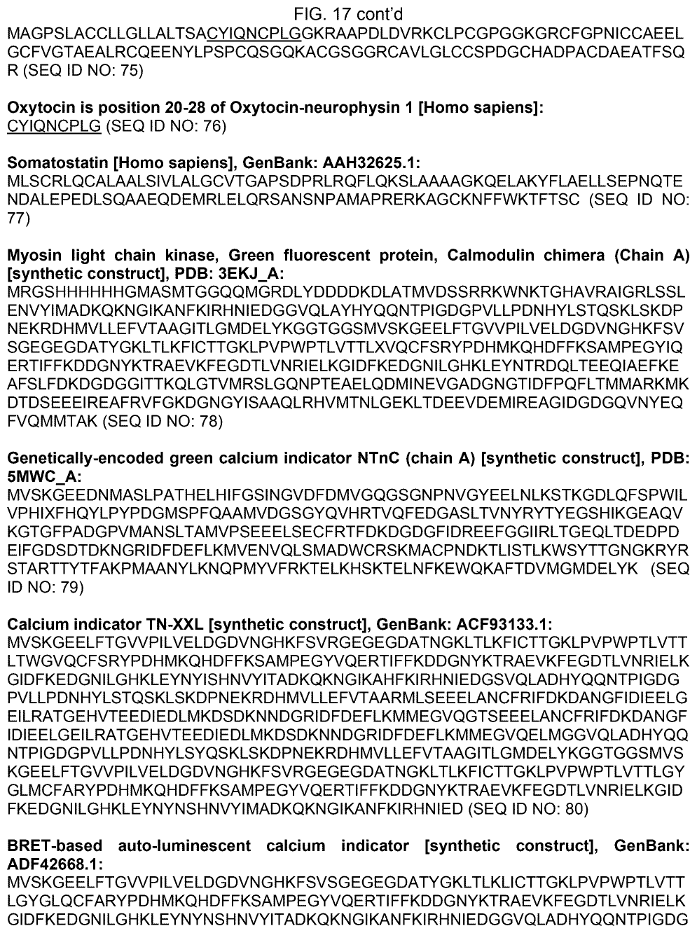

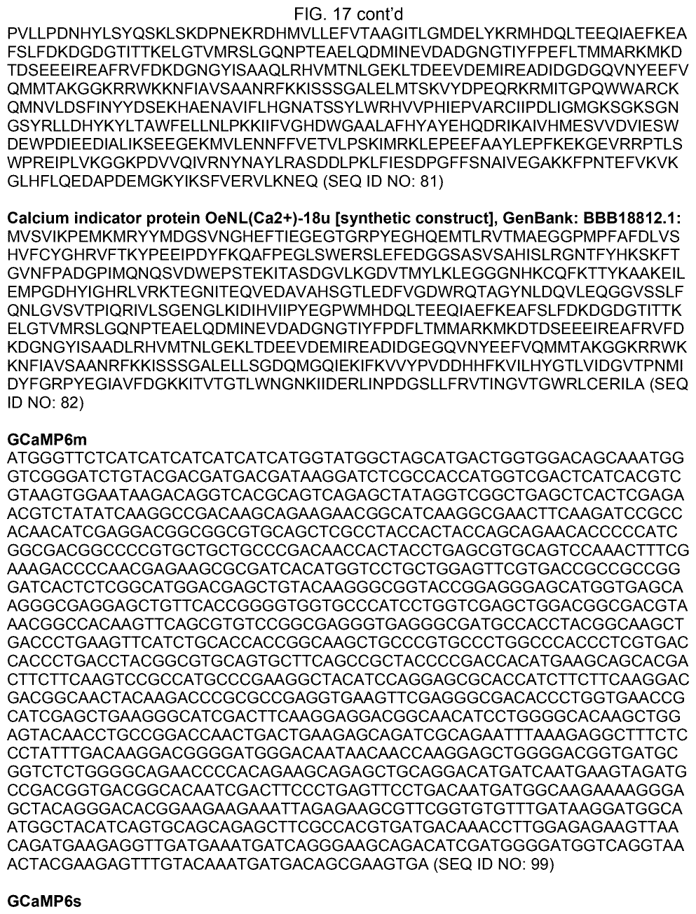

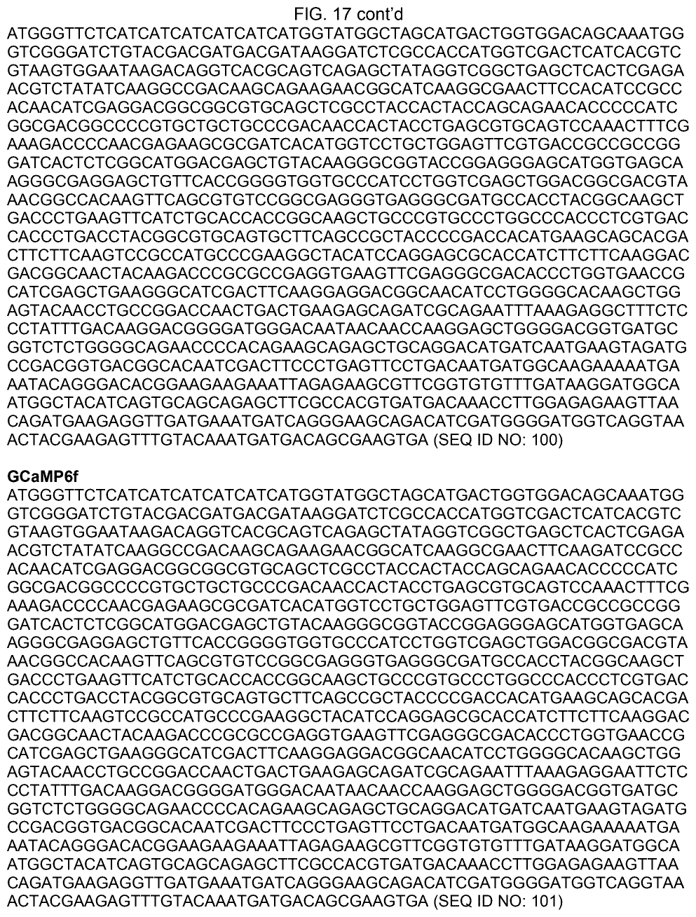

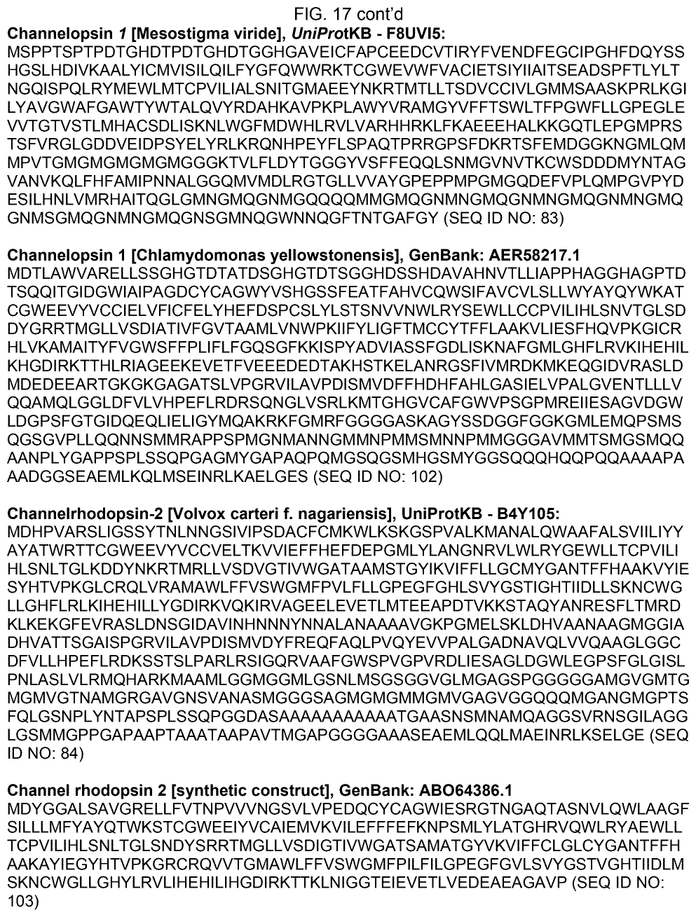

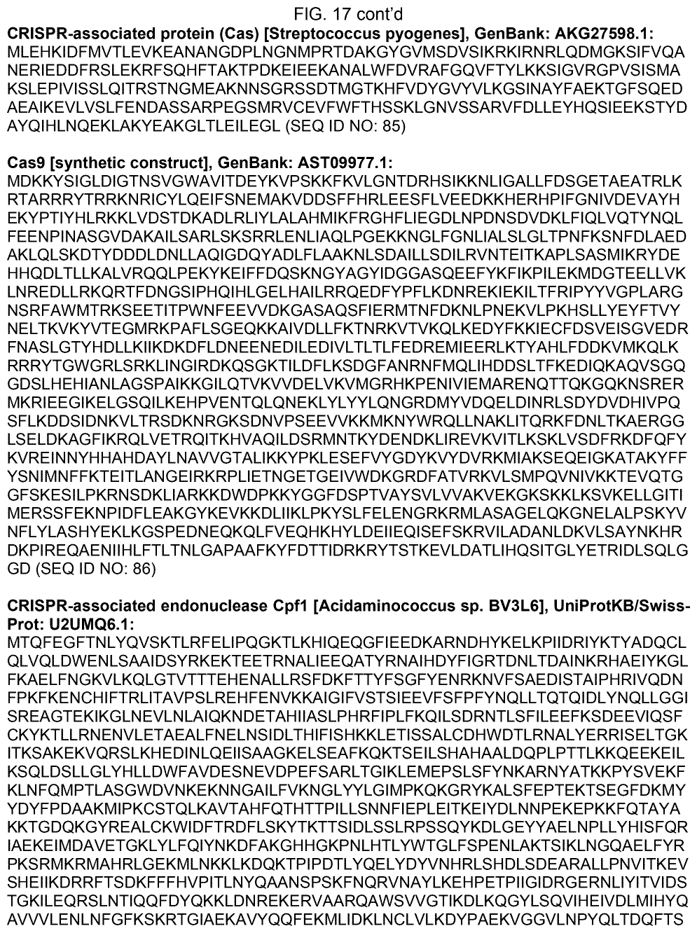

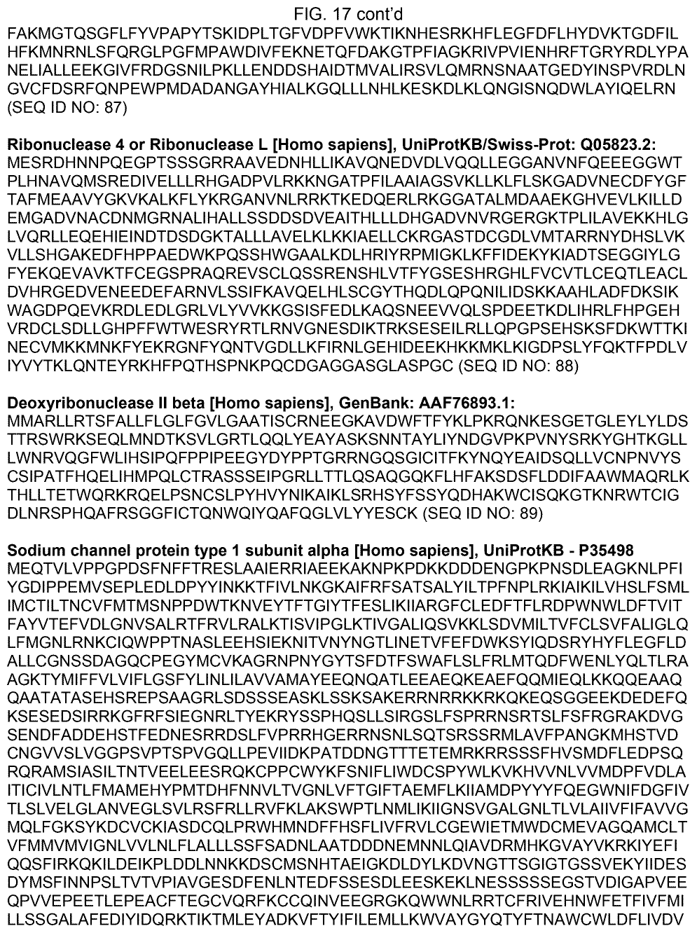

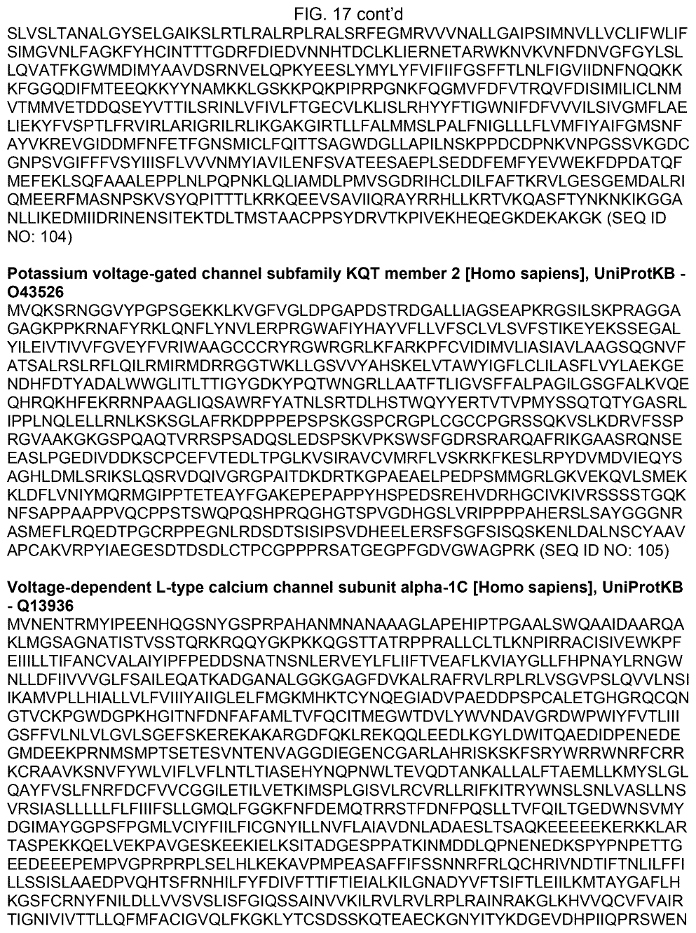

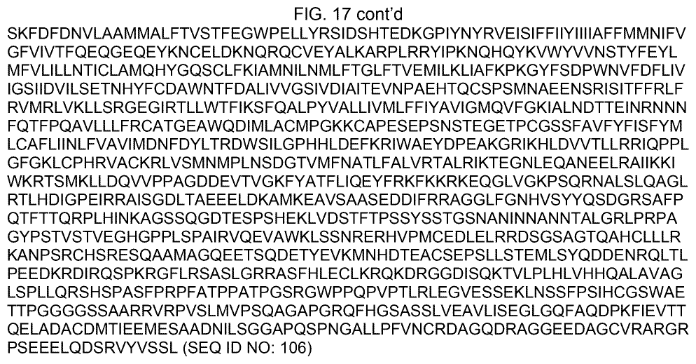

In particular embodiments, SYFP2 within CN1390, CN1244, CN1389, CN1203, CN1367, CN1498, CN1499, CN1500, and CN1838 can be replaced with a channel rhodopsin or calcium reporter, such as ChR2 or GCaMP. In particular embodiments, SYFP2 within CN1390 is replaced with ChR2 or GCaMP. 3×zI46i within CN1838 refers to the 3× concatemer of the zebrafish I46iCore. See also FIG. 16 which provides additional exemplary vector components and combinations of the disclosure.

In particular embodiments vectors (e.g., AAV) with capsids that cross the blood-brain barrier (BBB) are selected. In particular embodiments, vectors are modified to include capsids that cross the BBB. Examples of AAV with viral capsids that cross the blood brain barrier include AAV9 (Gombash et al., Front Mol Neurosci. 2014; 7:81), AAVrh. 10 (Yang, et al., Mol Ther. 2014; 22(7):1299-1309), AAV1R6, AAV1R7 (Albright et al., Mol Ther. 2018; 26(2) 510), rAAVrh.8 (Yang, et al., supra), AAV-BR1 (Marchio et al., EMBO Mol Med. 2016; 8(6):592), AAV-PHP.S (Chan et al., Nat Neurosci. 2017; 20(8):1172), AAV-PHP.B (Deverman et al., Nat Biotechnol. 2016; 34(2):204), and AAV-PPS (Chen et al., Nat Med. 2009; 15:1215). The PHP.eB capsid differs from AAV9 such that, using AAV9 as a reference, amino acids starting at residue 586: S-AQ-A (SEQ ID NO: 98) are changed to S-DGTLAVPFK-A (SEQ ID NO: 33).

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31(4):317), for example, as described in relation to clinical trials for the treatment of superior mesenteric artery (SMA) syndrome by AveXis (AVXS-101, NCT03505099) and the treatment of CLN3 gene-Related Neuronal Ceroid-Lipofuscinosis (NCT03770572).

AAVrh. 10, was originally isolated from rhesus macaques and shows low seropositivity in humans when compared with other common serotypes used for gene delivery applications (Selot et al., Front Pharmacol. 2017; 8:441) and has been evaluated in clinical trials LYS-SAF302, LYSOGENE, and NCT03612869.

AAV1R6 and AAV1R7, two variants isolated from a library of chimeric AAV vectors (AAV1 capsid domains swapped into AAVrh. 10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 91) epitope that was isolated during in vivo screening of a random AAV display peptide library. It shows high specificity accompanied by high transgene expression in the brain with minimal off-target affinity (including for the liver) (Körbelin et al., EMBO Mol Med. 2016; 8(6):609).

AAV-PHP.S (Addgene, Watertown, MA) is a variant of AAV9 generated with the CREATE method that encodes the 7-mer sequence QAVRTSL (SEQ ID NO: 92), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 93). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 94) epitope into the capsid of AAV2, shows a dramatically improved brain tropism relative to AAV2.

For additional information regarding capsids that cross the blood brain barrier, see Chan et al., Nat. Neurosci. 2017 Aug: 20(8):1172-1179.

### (ii) COMPOSITIONS FOR ADMINISTRATION

Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in particular embodiments, when administered intravenously (e.g. at the retro-orbital plexus).

In particular embodiments, compositions can be formulated for intravenous, intraocular, intravitreal, parenteral, subcutaneous, intracerebro-ventricular, intramuscular, intrathecal, intraspinal, oral, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24(12):1113-1128, 1998; Quintanar-Guerrero et al., Pharm Res. 15(7):1056-1062, 1998; Quintanar-Guerrero et al., J. Microencapsul. 15(1):107-119, 1998; Douglas et al., Crit Rev Ther Drug Carrier Syst 3(3):233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69(2):199-202, 1980; Couvreur et al., Crit Rev Ther Drug Carrier Syst. 5(1)1-20, 1988; zur Muhlen et al., Eur J Pharm Biopharm, 45(2):149-155, 1998; Zambaux et al., J Control Release 50(1-3):31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17(1):33-58, 1998), transdermal matrices (U.S. Pat. No. 5,770,219 and U.S. Pat. No. 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.

### (iii) CELL LINES INCLUDING ARTIFICIAL EXPRESSION CONSTRUCTS

The present disclosure includes cells including an artificial expression construct described herein. A cell that has been transformed with an artificial expression construct can be used for many purposes, including in neuroanatomical studies, assessments of functioning and/or non-functioning proteins, and drug screens that assess the regulatory properties of enhancers.

A variety of host cell lines can be used, but in particular embodiments, the cell is a mammalian neural cell. In particular embodiments, the enhancer sequence of the artificial expression construct is SEQ ID NO: 3 and/or 7 and/or a CN1390, CN1244, CN1389, CN1203, CN1367, CN1498, CN1499, CN1500, CN1838 or a combination of components depicted in FIG. 16, and the cell line is a human, primate, or murine neural cell. Cell lines which can be utilized for transgenesis in the present disclosure also include primary cell lines derived from living tissue such as rat or mouse brains and organotypic cell cultures, including brain slices from animals such as rats or mice. The PC12 cell line (available from the American Type Culture Collection, ATCC, Manassas, VA) has been shown to express a number of neuronal marker proteins in response to Neuronal Growth Factor (NGF). The PC12 cell line is considered to be a neuronal cell line and is applicable for use with this disclosure. JAR cells (available from ATCC) are a platelet derived cell-line that express some neuronal genes, such as the serotonin transporter gene, and may be used with embodiments described herein.

WO 91/13150 describes a variety of cell lines, including neuronal cell lines, and methods of producing them. Similarly, WO 97/39117 describes a neuronal cell line and methods of producing such cell lines. The neuronal cell lines disclosed in these patent applications are applicable for use in the present disclosure.

In particular embodiments, a “neural cell” refers to a cell or cells located within the central nervous system, and includes neurons and glia, and cells derived from neurons and glia, including neoplastic and tumor cells derived from neurons or glia. A “cell derived from a neural cell” refers to a cell which is derived from or originates or is differentiated from a neural cell.

In particular embodiments, “neuronal” describes something that is of, related to, or includes, neuronal cells. Neuronal cells are defined by the presence of an axon and dendrites. The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

In particular embodiments, non-neuronal cell lines may be used, including mouse embryonic stem cells. Cultured mouse embryonic stem cells can be used to analyze expression of genetic constructs using transient transfection with plasmid constructs. Mouse embryonic stem cells are pluripotent and undifferentiated. These cells can be maintained in this undifferentiated state by Leukemia Inhibitory Factor (LIF). Withdrawal of LIF induces differentiation of the embryonic stem cells. In culture, the stem cells form a variety of differentiated cell types. Differentiation is caused by the expression of tissue specific transcription factors, allowing the function of an enhancer sequence to be evaluated. (See for example Fiskerstrand et al., FEBS Lett 458:171-174, 1999.)

Methods to differentiate stem cells into neuronal cells include replacing a stem cell culture media with a media including basic fibroblast growth factor (bFGF) heparin, an N2 supplement (e.g., transferrin, insulin, progesterone, putrescine, and selenite), laminin and polyornithine. A process to produce myelinating oligodendrocytes from stem cells is described in Hu, et al., 2009, Nat. Protoc. 4:1614-22. Bibel, et al., 2007, Nat. Protoc. 2:1034-43 describes a protocol to produce glutamatergic neurons from stem cells while Chatzi, et al., 2009, Exp. Neurol. 217:407-16 describes a procedure to produce GABAergic neurons. This procedure includes exposing stem cells to all-trans-RA for three days. After subsequent culture in serum-free neuronal induction medium including Neurobasal medium supplemented with B27, bFGF and EGF, 95% GABA neurons develop

U.S. Publication No. 2012/0329714 describes use of prolactin to increase neural stem cell numbers while U.S. Publication No. 2012/0308530 describes a culture surface with amino groups that promotes neuronal differentiation into neurons, astrocyles and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No.5,766,948; FGF-1, FGF-2); Neurotrophin-3 (NT-3) and Neurotrophin-4 (NT-4); Caldwell, et al., 2001, Nat. Biotechnol. 1;19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-αand TGF-β (U.S. Pat. Nos. 5,851,832 and 5,753,506).

In particular embodiments, yeast one-hybrid systems may also be used to identify compounds that inhibit specific protein/DNA interactions, such as transcription factors for I56i enhancers, cores thereof and/or the SEQ ID NO: 3 and/or 7

Transgenic animals are described below. Cell lines may also be derived from such transgenic animals. For example, primary tissue culture from transgenic mice (e.g., also as described below) can provide cell lines with the expression construct already integrated into the genome. (for an example see Mackenzie & Quinn, Proc Natl Acad Sci USA 96:15251-15255, 1999).

### (iv) TRANSGENIC ANIMALS

Another aspect of the disclosure includes transgenic animals, the genome of which contains an artificial expression construct including concatamerizations of I56i enhancer cores such as SEQ ID NO: 2 and/or 6 (e.g., SEQ ID NO: 3 and/or 7) operatively linked to a heterologous coding sequence. In particular embodiments, the genome of a transgenic animal includes the CN1390, CN1244, CN1389, CN1203, CN1367, CN1498, CN1499, CN1500, CN1838 or a combination of components depicted in FIG. 16. In particular embodiments, when a non-integrating vector is utilized, a transgenic animal includes an artificial expression construct including concatemerizations of I56i enhancer cores such as SEQ ID NO: 2 and/or 6 (e.g., SEQ ID NO: 3 and/or 7) and/or CN1390, CN1244, CN1389, CN1203, CN1367, CN1498, CN1499, CN1500, CN1838 or a combination of components depicted in FIG. 16 within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In particular embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to heterologous expression of the construct. A disadvantage of this approach is that the expression of the introduced DNA may be affected by the specific genetic background of the host animal.

As indicated above in relation to cell lines, the artificial expression constructs of this disclosure can be used to genetically modify mouse embryonic stem cells using techniques known in the art. Typically, the artificial expression construct is introduced into cultured murine embryonic stem cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice be crossed with an appropriate strain to produce offspring that will carry the transgene.

In addition to the methods of delivery described above, the following techniques are also contemplated as alternative methods of delivering artificial expression constructs to target cells or selected tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17(1):33-58, 1998); transdermal matrices (U.S. Pat. Nos. 5,770,219 and 5,783,208); and feedback-controlled delivery (U.S. Pat. No. 5,697,899).

### (v) METHODS OF USE

In particular embodiments, a composition including a physiologically active component described herein is administered to a subject to result in a physiological effect.

In particular embodiments, the disclosure includes the use of the artificial expression constructs described herein to modulate expression of a heterologous gene which is either partially or wholly encoded in a location downstream to that enhancer in an engineered sequence. Thus, there are provided herein methods of use of the disclosed artificial expression constructs in the research, study, and potential development of medicaments for preventing, treating or ameliorating the symptoms of a disease, dysfunction, or disorder.

Particular embodiments include methods of administering to a subject an artificial expression construct that includes SEQ ID NO: 2, SEQ ID NO: 6, SEQ ID NO: 3 and/or SEQ ID NO: 7 as described herein to drive selective expression of a gene in a selected neural cell type.

Particular embodiments include methods of administering to a subject an artificial expression construct that includes CN1390, CN1244, CN1389, CN1203, CN1367, CN1498, CN1499, CN1500, CN1838 or a combination of components depicted in FIG. 16 as described herein to drive selective expression of a gene in a selected neural cell type wherein the subject can be an isolated cell, a network of cells, a tissue slice, an experimental animal, a veterinary animal, or a human.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in particular embodiments, a dose could be from 10⁵ to 10¹⁰⁰ copies of an artificial expression construct of the disclosure. In particular embodiments, a patient receiving intravenous, intraspinal, retro-orbital, or intrathecal administration can be infused with from 10⁶ to 10²² copies of the artificial expression construct.

An “effective amount” is the amount of a composition necessary to result in a desired physiological change in the subject. Effective amounts are often administered for research purposes. Effective amounts disclosed herein can cause a statistically-significant effect in an animal model or in vitro assay.

In particular embodiments, constructs disclosed herein can be utilized to treat Dravet Syndrome. In particular embodiments, the methods reduce or prevent seizures, or symptoms thereof in a patient in need thereof. In particular embodiments, the methods provided may reduce or prevent one or more different types of seizures. Ideally, the methods of the disclosure result in a total prevention of seizures. However, the disclosure also encompasses methods in which the instances of seizures are decreased by at least 10%, at least 20%, at least 30%, at least 40%, at least 50%, at least 60%, at least 70%, at least 80% or at least 90%.

Generally, a seizure can include convulsions, repetitive movements, unusual sensations, and combinations thereof. Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures.

In particular embodiments, methods described herein may reduce the frequency of seizures, reduce the severity of seizures, change the type of seizures (e.g., from a more severe type to a less severe type), or a combination thereof in a patient after treatment compared to the absence of treatment (e.g., before treatment), or compared to treatment with an alternative conventional treatment.

The amount of expression constructs and time of administration of such compositions will be within the purview of the skilled artisan having benefit of the present teachings. It is likely, however, that the administration of effective amounts of the disclosed compositions may be achieved by a single administration, such as for example, a single injection of sufficient numbers of infectious particles to provide an effect in the subject. Alternatively, in some circumstances, it may be desirable to provide multiple, or successive administrations of the artificial expression construct compositions or other genetic constructs, either over a relatively short, or a relatively prolonged period of time, as may be determined by the individual overseeing the administration of such compositions. For example, the number of infectious particles administered to a mammal may be 10⁷, 10⁸, 10⁹, 10¹⁰, 10¹¹, 10¹², 10¹³, or even higher, infectious particles/ml given either as a single dose or divided into two or more administrations as may be required to achieve an intended effect. In fact, in certain embodiments, it may be desirable to administer two or more different expression constructs in combination to achieve a desired effect.

In certain circumstances it will be desirable to deliver the artificial expression construct in suitably formulated compositions disclosed herein either by pipette, retro-orbital injection, subcutaneously, intraocularly, intravitreally, parenterally, subcutaneously, intravenously, intracerebro-ventricularly, intramuscularly, intrathecally, intraspinally, orally, intraperitoneally, by oral or nasal inhalation, or by direct application or injection to one or more cells, tissues, or organs. The methods of administration may also include those modalities as described in U.S. Pat. Nos. 5,543,158; 5,641,515 and 5,399,363.

(vi) Kits and Commercial Packages. Kits and commercial packages contain an artificial expression construct described herein. The expression construct can be isolated. In particular embodiments, the components of an expression product can be isolated from each other. In particular embodiments, the expression product can be within a vector, within a viral vector, within a cell, within a tissue slice or sample, and/or within a transgenic animal. Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition.

The Exemplary Embodiments and Experimental Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (vii) EXEMPLARY EMBODIMENTS

1. A core of a I56i enhancer, a concatemerized core of a I56i enhancer, or a concatemerized I56i enhancer.
2. The I56i enhancer core, concatemerized I56i enhancer core, or concatemerized I56i enhancer of embodiment 1, wherein the I56i enhancer is human, murine, or zebrafish (I46i).
3. The I56i enhancer core, concatemerized I56i enhancer core, or concatemerized I56i enhancer of embodiment 1 or 2, wherein the concatemerized core includes SEQ ID NO: 2 or 6.
4. The I56i enhancer core, concatemerized I56i enhancer core, or concatemerized I56i enhancer of any of embodiments 1-3, wherein the concatemerized core includes 2, 3, 4, 5, 6, 7, 8, 9, or 10 copies of the I56i core.
5. The I56i enhancer core, concatemerized I56i enhancer core, or concatemerized I56i enhancer of embodiment 4, including 2, 3, 4, 5, 6, 7, 8, 9, or 10 copies of SEQ ID NO: 2 and/or 6 (e.g., SEQ ID NO: 2 and SEQ ID NO: 6 within one sequence such as SEQ ID NO: 2-SEQ ID NO: 2-SEQ ID NO: 6; SEQ ID NO: 2-SEQ ID NO: 6-SEQ ID NO: 6; SEQ ID NO: 2-SEQ ID NO: 6-SEQ ID NO: 2; SEQ ID NO: 6-SEQ ID NO: 6-SEQ ID NO: 2; SEQ ID NO: 6-SEQ ID NO: 2-SEQ ID NO: 2; and SEQ ID NO: 6-SEQ ID NO: 2-SEQ ID NO: 6).
6. The I56i enhancer core, concatemerized I56i enhancer core, or concatemerized I56i enhancer of embodiment 4 or 5, including 2, 3, 4, 5, 6, 7, 8, 9, or 10 copies of SEQ ID NO: 2.
7. The I56i enhancer core, concatemerized I56i enhancer core, or concatemerized I56i enhancer of embodiment 4 or 5, including 2, 3, 4, 5, 6, 7, 8, 9, or 10 copies of SEQ ID NO: 6.
8. The I56i enhancer core, concatemerized I56i enhancer core, or concatemerized I56i enhancer of embodiment 4 or 5, including 3 copies of SEQ ID NO: 2.
9. The I56i enhancer core, concatemerized I56i enhancer core, or concatemerized I56i enhancer of embodiment 4 or 5, including 3 copies of SEQ ID NO: 6.
10. The I56i enhancer core, concatemerized I56i enhancer core, or concatemerized I56i enhancer of embodiment 8, wherein the concatemerized core includes SEQ ID NO: 3.
11. The I56i enhancer core, concatemerized I56i enhancer core, or concatemerized I56i enhancer of embodiment 9, wherein the concatemerized core includes SEQ ID NO: 7.
12. An artificial expression construct including (i) an I56i enhancer core, concatemerized I56i enhancer core, or concatemerized I56i enhancer of any of embodiments 1-11, (ii) a promoter; and (iii) a heterologous encoding sequence.
13. The artificial expression construct of embodiment 12, wherein the heterologous encoding sequence encodes an effector element or an expressible element.
14. The artificial expression construct of embodiment 12 or 13, wherein the effector element includes a reporter protein or a functional molecule.
15. The artificial expression construct of embodiment 14, wherein the reporter protein is a fluorescent protein.
16. The artificial expression construct of embodiment 14 or 15, wherein the effector element is Cre, iCre, dgCre, FIpE, FIpO, or tTA2 or a functional molecule selected from a functional ion transporter, enzyme, a transcription factor, a receptor, a membrane protein, a cellular trafficking protein, a signaling molecule, a neurotransmitter, a calcium reporter, a channel rhodopsin, a CRISPR/CAS molecule, an editase, a guide RNA molecule, a homologous recombination donor cassette, or a designer receptor exclusively activated by designer drug (DREADD).
17. The artificial expression construct of any of embodiments 13, wherein the expressible element is a non-functional molecule.
18. The artificial expression construct of embodiment 17, wherein the non-functional molecule is a non-functional ion transporter, enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or a DREADD.
19. The artificial expression construct of any of embodiments 12-18, wherein the expression construct is associated with a capsid that crosses the blood brain barrier.
20. The artificial expression construct of embodiment 19, wherein the capsid includes PHP.eB, AAV-BR1, AAV-PHP.S, AAV-PHP.B, or AAV-PPS.
21. The artificial expression construct of any of embodiments 12-20, wherein the expression construct includes or encodes a skipping element.
22. The artificial expression construct of embodiment 21, wherein the skipping element includes a 2A peptide and/or an internal ribosome entry site (IRES).
23. The artificial expression construct of embodiment 22, wherein the 2A peptide is selected from T2A, P2A, E2A, or F2A.
24. The artificial expression construct of any of embodiments 12-23, wherein the expression construct includes a set of features selected from: 3×hI56Core, minBglobin, minCMV, SYFP2, His, 3×HA, NavMs, NavBp, NavSheP-D60N, WPRE3, BGHpA or a combination of features selected from a construct depicted in FIG. 16.
25. A vector including an artificial expression construct of any of embodiments 12-24.
26. A vector including a combination of components depicted in FIG. 16.
27. The vector of embodiment 26, wherein the vector is a viral vector.
28. The vector of embodiment 26 or 27, wherein the viral vector is a recombinant adeno- associated viral (AAV) vector.
29. An adeno-associated viral (AAV) vector including at least one heterologous encoding sequence, wherein the heterologous encoding sequence is under control of a promoter and an enhancer selected from SEQ ID NO: 3 and/or 7.
30. The AAV vector of embodiment 29, wherein the AAV vector is replication-competent.
31. A transgenic cell including an expression construct or vector of any of the preceding embodiments.
32. The transgenic cell of embodiment 31, wherein the transgenic cell is a GABAergic interneuron.
33. A non-human transgenic animal including an expression construct, vector, or transgenic cell of any of the preceding embodiments.
34. The non-human transgenic animal of embodiment 33 wherein the non-human transgenic animal is a mouse or a non-human primate.
35. An administrable composition including an expression construct, vector, or transgenic cell of any of the preceding embodiments.
36. A kit including an expression construct, vector, transgenic cell, transgenic animal, and/or administrable compositions of any of the preceding embodiments.
37. A method for selectively expressing a heterologous gene within a population of neural cells in vivo or in vitro, the method including providing the administrable composition of embodiment 35 in a sufficient dosage and for a sufficient time to a sample or subject including the population of neural cells thereby selectively expressing the gene within the population of neural cells.
38. The method of embodiment 37, wherein the heterologous gene encodes an effector element or an expressible element.
39. The method of embodiment 38, wherein the effector element includes a reporter protein or a functional molecule.
40. The method of embodiment 39, wherein the reporter protein is a fluorescent protein.
41. The method of embodiment 39 or 40, wherein the effector element is Cre, iCre, dgCre, FlpE, FlpO, or tTA2 or a functional molecule selected from a functional ion transporter, enzyme, a transcription factor, a receptor, a membrane protein, a cellular trafficking protein, a signaling molecule, a neurotransmitter, a calcium reporter, a channel rhodopsin, a CRISPR/CAS molecule, an editase, a guide RNA molecule, a homologous recombination donor cassette, or a DREADD.
42. The method of embodiment 38, wherein the expressible element is a non-functional molecule.
43. The method of embodiment 42, wherein the non-functional molecule is a non-functional ion transporter, enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or DREADD.
44. The method of any of embodiments 37-43, wherein the providing includes pipetting.
45. The method of embodiment 44, wherein the pipetting is to a brain slice.
46. The method of embodiment 45, wherein the brain slice includes a GABAergic interneuron.
47. The method of any of embodiments 45 or 46, wherein the brain slice is murine, human, or non-human primate.
48. The method of any of embodiments 37-43, wherein the providing includes administering to a living subject.
49. The method of embodiment 48, wherein the living subject is a human, non-human primate, or a mouse.
50. The method of any of embodiments 48 or 49, wherein the administering to a living subject is through injection.

51. The method of embodiment 50, wherein the injection includes intravenous injection, intraparenchymal injection into brain tissue, intracerebroventricular (ICV) injection, intra-cisterna magna (ICM) injection, or intrathecal injection.

52. An artificial expression construct consisting of or consisting essentially of a combination of features depicted in FIG. 16.

53. Any of the embodiments above including an effector element or expressible element wherein the effector element or expressible element is an ion transporter selected from a voltage gated sodium channel (e.g., SCN1A), a potassium channel (e.g., KCNQ2), or a calcium channel (e.g., CACNA1C); a cellular trafficking protein selected from clathrin, dynamin, caveolin, Rab-4A, or Rab-11A; an enzyme selected from lactase, lipase, helicase, alpha-glucosidase, and amylase); a transcription factor selected from SP1, AP-1, Heat shock factor protein 1, C/EBP (CCAA-T/enhancer binding protein), and Oct-1; a receptor selected from transforming growth factor receptor beta 1, platelet-derived growth factor receptor, epidermal growth factor receptor, vascular endothelial growth factor receptor, and interleukin 8 receptor alpha; a signaling molecule selected from nerve growth factor (NGF), platelet-derived growth factor (PDGF), transforming growth factor B (TGFB), epidermal growth factor (EGF), and GTPase HRas; a neurotransmitter selected from cocaine and amphetamine regulated transcript, substance P, oxytocin, and somatostatin; a calcium reporter selected from genetically encoded calcium indicators (GECI, NTnC, GCaMP6s, GCaMP6f, GCaMP6m, jGCaMP7s, jGCaMP7f, jGCaMP7b, jGCaMP7c, jRGECO1a, jRGECO1b), Myosin light chain kinase, Green fluorescent protein, Calmodulin chimera, Calcium indicator TN-XXL, BRET-based auto-luminescent calcium indicator, and Calcium protein OeNL (Ca2+)-18u); a channel rhodopsin selected from channelrhodopsin-1 and channelrhodopsin-2 or a variant thereof; guide RNA; a nuclease selected from Cas, Cas9, Cpf1, ribonuclease 4, and deoxyribonuclease Il beta; and/or a DREADD (e.g., hM3DREADD, hM4DREADD).

Within this disclosure I56i should be interpreted to be I46i when the context demonstrates reference to or use of the zebrafish form of the enhancers described herein

### (viii) EXPERIMENTAL EXAMPLE

Dravet syndrome (DS) is a drug-resistant and life-threatening form of epilepsy. It typically begins in the first year of life, with fever- or temperature-induced seizures that evolve into generalized clonic, tonic-clonic, and unilateral seizures. These seizures are often resistant to current anti-epileptic drugs, the first-line therapies for this syndrome; complete seizure control is typically not achieved. As the disease progresses, most affected children also suffer from comorbid conditions including developmental delays, intellectual disabilities, impaired motor control and coordination, autistic behaviors, sleep disturbances, and many die prematurely.

Heterozygous loss-of-function mutations in SCN1A, the gene that encodes the pore-forming subunit of the voltage-gated sodium channel Nav1.1 are the most common cause of DS and occur in nearly 1/16,000 newborns.

A mouse model, generated by knock-out of Scn1a, replicates the several key phenotypic features of this epilepsy including infantile (P21)-epilepsy onset, high susceptibility to thermal seizures, ataxia, spontaneous seizures, sleep impairments, autistic behaviors, and premature death. Seizures and several comorbidities arise from impaired interneuron function in these mice.

This mouse model was used to investigate the efficacy of a new viral vector for DS. The virus was delivered by retro-orbital injection using an insulin syringe and its ability to suppress seizure was evaluated using the thermal seizure test. In this test, the mouse body core temperature is elevated slowly, using a temperature controller and a heat lamp, until a seizure occurs, or 42.5° C. is attained. The temperature of seizure onset in treated and control mice are compared to determine the efficacy of the intervention. In additional tests, the efficacy of treatment on spontaneous seizure and premature mortality are assessed using video and electroencephalographic monitoring.

The viral vector is a new AAV viral vector named CN1500. This viral vector is a recombinant AAV that expresses the transgene SYFP2-P2A-NavSheP-D60N to rescue the loss of the voltage-gated sodium channel Nav1.1. NavSheP-D60N is a modified voltage-gated sodium channel of bacterial origin that has been modified to improve the kinetics and expression in mammalian cells. The transgene expression level is elevated by the addition of a WPRE3 element, and transcription is terminated with the bovine growth hormone poly adenylation sequence. Expression of the transgene is high and limited to inhibitory cells in forebrain structures including the cortex and the hippocampus, via the 3×hI56iCore synthetic enhancer (SEQ ID NO: 3) directly 5′ of a CMV minimal promoter. Furthermore, the therapeutic transgene NavSheP-D60N is labeled by an HA epitope tag to verify correct protein localization.

To test the efficacy of the therapeutic AAV viral vector, CN1500 package using the PHP.eB serotype was used. A cohort of postnatal day 35 Scn1a^{+/-} mice were either injected with 2×10¹¹ vg per animal or were left un-injected. The AAV was introduced intravenously using the retro-orbital delivery route. Two weeks after viral administration, animals from the treatment and control groups were assessed for their susceptibility to febrile seizures. As indicated previously, febrile seizures were measured by steadily raising the mouse's temperature under a heat lamp 0.5 Celsius every two minutes and measuring the internal temperature of the mouse with a rectal probe. The temperature where the mouse experienced a seizure is recorded.

The new therapeutic vector CN1500 was both highly expressed in mouse cortical and hippocampal GABAergic cells, but also raised the average temperature where Scn1a^{+/-} mice experienced febrile seizures from 38.7° C. to 41° C. These data show that CN1500 can substantially rescue the loss of Scn1a.

Example 1 references include: Catterall et al. (2010) The Journal of physiology 588:1849-1859; Cheah et al. (2012) Proceedings of the National Academy of Sciences of the United States of America 109:14646-14651; Kalume (2013) Respir Physiol Neurobiol. 189(2):324-8; Kalume et al., (2007) J Neurosci 27:11065-11074; Kalume et al., (2013) The Journal of clinical investigation 123:1798-1808; Oakley et al., (2009) Proceedings of the National Academy of Sciences of the United States of America 106:3994-3999.

### (ix) CLOSING PARAGRAPHS

Nucleic acid sequences described herein are shown using standard letter abbreviations for nucleotide bases, as defined in 37 C.F.R. § 1.822. Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and lle; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W. H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (-−.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0+1); Ser (+0.3); Asn (+0.2); Gln (+0.2); Gly (0); Thr (−0.4); Pro (−0.5+1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in selective expression in the targeted cell population as determined by scRNA-Seq and the following enhancer/targeted cell population pairing: concatemerized core of the I56i enhancer (e.g., SEQ ID NO: 3)/GABAergic interneurons.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1.-51. (canceled)

52. A method for expressing a coding sequence within a targeted population of cells in vivo, the method comprising administering to a living subject an administrable composition comprising an artificial expression construct comprising:
i) a concatemer comprising 2, 3, 4, 5, 6, 7, 8, 9, or 10 copies of a sequence consisting of SEQ ID NO: 2 or SEQ ID NO: 6;
ii) a promoter; and
iii) a coding sequence,
in a sufficient dosage and for a sufficient time to the living subject comprising the population of cells thereby expressing the coding sequence within the population of cells.

53. The method of claim 52, wherein the coding sequence encodes a fluorescent protein or an ion transporter selected from a potassium channel or a calcium channel.

54. The method of claim 52, wherein the coding sequence encodes an enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or DREADD.

55. The method of claim 52, wherein the living subject is a human, non-human primate, or a mouse.

56. The method of claim 52, wherein the administering is through injection.

57. The method of claim 56, wherein the injection comprises intravenous injection, intraparenchymal injection into brain tissue, intracerebroventricular (ICV) injection, intra-cisterna magna (ICM) injection, or intrathecal injection.

58. A method for expressing a coding sequence within a targeted population of cells in vitro, the method comprising providing an administrable composition comprising an artificial expression construct comprising:
i) a concatemer comprising 2, 3, 4, 5, 6, 7, 8, 9, or 10 copies of a sequence consisting of SEQ ID NO: 2 or SEQ ID NO: 6;
ii) a promoter; and
iv) a coding sequence,
in a sufficient dosage and for a sufficient time to a sample comprising the population of cells thereby expressing the coding sequence within the population of cells.

59. The method of claim 58, wherein the coding sequence encodes a fluorescent protein or an ion transporter selected from a potassium channel or a calcium channel.

60. The method of claim 58, wherein the coding sequence encodes an enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or DREADD.

61. The method of claim 58, wherein the providing comprises pipetting to a brain slice.

62. The method of claim 61, wherein the brain slice includes a GABAergic interneuron.

63. The method of claim 61, wherein the brain slice includes a lysosomal associated membrane protein 5 (LAMP5) neuron, a vasoactive intestinal peptide (Vip) neuron, a somatostatin (Sst) neuron, or a parvalbumin (Pvalb) neuron.

64. A transgenic cell comprising an artificial expression construct comprising:
i) a concatemer comprising 2, 3, 4, 5, 6, 7, 8, 9, or 10 copies of a sequence consisting of SEQ ID NO: 2 or SEQ ID NO: 6;
ii) a promoter; and
iii) a coding sequence.

65. The transgenic cell of claim 64, wherein the transgenic cell is a GABAergic interneuron.

66. The transgenic cell of claim 64, wherein the transgenic cell is a lysosomal associated membrane protein 5 (LAMP5) neuron, a vasoactive intestinal peptide (Vip) neuron, a somatostatin (Sst) neuron, or a parvalbumin (Pvalb) neuron.

67. The transgenic cell of claim 64, wherein the transgenic cell is murine, human, or non-human primate.

68. The transgenic cell of claim 64, wherein the coding sequence encodes a fluorescent protein or an ion transporter selected from a potassium channel or a calcium channel.

69. The transgenic cell of claim 64, wherein the concatemer has 3 copies of the sequence consisting of SEQ ID NO: 2 or 3 copies of the sequence consisting of SEQ ID NO: 6.

70. The transgenic cell of claim 64, wherein the artificial expression construct is associated with a capsid that crosses a blood brain barrier.

71. The transgenic cell of claim 64, wherein the artificial expression construct is within a viral vector.


SEQUENCE LISTING (106 sequences)

SEQ ID NO: 1 - DNA, 528 bp, Homo sapiens
  source 1..528: mol_type = genomic DNA
tatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagg

SEQ ID NO: 2 - DNA, 131 bp, Homo sapiens
  source 1..131: mol_type = genomic DNA
ctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 3 - DNA, 393 bp, synthetic construct
  misc_feature 1..393: note = Triply concatamerized core of Homo spaiens I56i Core
  source 1..393: mol_type = other DNA
ctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 4 - DNA, 527 bp, Mus musculus
  source 1..527: mol_type = genomic DNA
tatacactcacagtggtttggcatatatttggtgaaattttttaaggaaaaattagtgttggtttcgatatatggtagctttttctctaacataatttgaataattcagcaaagccctactaccagctgtacttctgcagcctcttccattctttccagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagg

SEQ ID NO: 5 - DNA, 281 bp, Danio rerio
  source 1..281: mol_type = genomic DNA
acattgtaattttagataatatcccaagcgttcactctcctcggcaatttgtacatgaataaccgaataatttcatcttttgtttcgtctttgccacttcaaatccaaataaagatgccttttagtattaaaagtggtagaaaattacaggtaattatctttgacggtaaaaacgctgtaatcagcgggctacatcaaaaattaccctaattatgtctgcatttatgagaatggaaaaaaaccctctcttggataaaacccataaattgtcccaaatatct

SEQ ID NO: 6 - DNA, 130 bp, Danio rerio
  source 1..130: mol_type = genomic DNA
ccaaataaagatgccttttagtattaaaagtggtagaaaattacaggtaattatctttgacggtaaaaacgctgtaatcagcgggctacatcaaaaattaccctaattatgtctgcatttatgagaatgg

SEQ ID NO: 7 - DNA, 390 bp, synthetic construct
  misc_feature 1..390: note = Triply concatamerized core of I56i enhancer from Danio rerio
  source 1..390: mol_type = other DNA
ccaaataaagatgccttttagtattaaaagtggtagaaaattacaggtaattatctttgacggtaaaaacgctgtaatcagcgggctacatcaaaaattaccctaattatgtctgcatttatgagaatggccaaataaagatgccttttagtattaaaagtggtagaaaattacaggtaattatctttgacggtaaaaacgctgtaatcagcgggctacatcaaaaattaccctaattatgtctgcatttatgagaatggccaaataaagatgccttttagtattaaaagtggtagaaaattacaggtaattatctttgacggtaaaaacgctgtaatcagcgggctacatcaaaaattaccctaattatgtctgcatttatgagaatgg

SEQ ID NO: 8 - DNA, 53 bp, synthetic construct
  misc_feature 1..53: note = Beta-Globin Minimal Promoter
  source 1..53: mol_type = other DNA
gggctgggcataaaagtcagggcagagccatctattgcttacatttgcttctg

SEQ ID NO: 9 - DNA, 68 bp, synthetic construct
  misc_feature 1..68: note = minCMV Promoter
  source 1..68: mol_type = other DNA
gaggtaggcgtgtacggtgggaggcctatataagcagagctcgtttagtgaaccgtcagatcgcctgg

SEQ ID NO: 10 - DNA, 68 bp, synthetic construct
  misc_feature 1..68: note = Mutated minCMV Promoter
  source 1..68: mol_type = other DNA
gaggtaggcgtgtacggtgggaggcctatataagcagagctggtttagtgaaccgtcagatcgcctgg

SEQ ID NO: 11 - DNA, 120 bp, synthetic construct
  misc_feature 1..120: note = minRho Promoter
  source 1..120: mol_type = other DNA
gattcagccgggagcttagggaggggaggtcacttcataagggcctggggggggagttggagccacgagtcgtccagccggagccccgtgtggctgagctccggcctcagaagcatcccc

SEQ ID NO: 12 - DNA, 867 bp, synthetic construct
  misc_feature 1..867: note = Hsp68 minimal Promoter
  source 1..867: mol_type = other DNA
caggaacatccaaactgagcagccggggtcccccccaccccccaccccgccccacgcggcaactttgagcctgtgctgggacagagcctctagttcctaaattagtccatgaggtcagaggcagcactgccattgtaacgcgattggagaggatcacgtcaccggacacgcccccaggcatctccctgggtctcctaaacttggcggggagaagttttagcccttaagttttagcctttaacccccatattcagaactgtgcgagttggcgaaaccccacaaatcacaacaaactgtacacaacaccgagctagaggtgatctttcttgtccattccacacaggccttagtaatgcgtcgccatagcaacagtgtcactagtagcaccagcacttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcaggtcaccagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgcc

SEQ ID NO: 13 - DNA, 720 bp, synthetic construct
  misc_feature 1..720: note = SYFP2
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 14 - DNA, 720 bp, synthetic construct
  misc_feature 1..720: note = enhanced green fluorescent protein (EGFP)
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 15 - DNA, 1299 bp, synthetic construct
  misc_feature 1..1299: note = Optimized Flp recombinase
  source 1..1299: mol_type = other DNA
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 16 - DNA, 1056 bp, synthetic construct
  misc_feature 1..1056: note = Improved Cre recombinase (iCre)
  source 1..1056: mol_type = other DNA
atggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaa

SEQ ID NO: 17 - DNA, 825 bp, Magnetococcus marinus
  source 1..825: mol_type = genomic DNA
atgtcacgcaaaataagagatttaatcgaatccaaacgctttcaaaacgtcatcaccgccattattgtgctcaatggcgctgtgctgggtctgctgaccgatacaaccctatcggcctccagccaaaacctgctggagcgtgtggatcaactttgtctgactatctttattgttgaaatatccctgaaaatatacgcctatggcgtgcgaggctttttccgcagcggctggaatctgtttgattttgtgattgtggccatcgcgcttatgcccgcccagggtagcctatcggtgctgcgaaccttccgtatattccgcgtcatgcggctcgtatcggtcataccaaccatgcgaagagtggtgcaaggcatgctcttggcactgcccggcgtgggatcggtagcggcactgttgacggtggtcttctatattgcggctgtcatggccaccaatctctacggggcaaccttccctgaatggtttggtgatcttagcaagagcctgtacacactatttcaggtgatgaccttagagtcatggtctatgggcattgtgcgtccagtgatgaacgttcatcccaacgcatgggtttttttcatccccttcatcatgctcaccacctttaccgtgctcaacctgtttattggcattattgtagatgccatggccatcaccaaggaacaggaggaagaggccaaaaccggccaccaccaagagcctattagccaaacattgctccatctgggagatcgcctagataggatcgaaaagcagcttgcgcaaaacaacgagctcttacaacgacaacagccgcaaaaaaaatag

SEQ ID NO: 18 - DNA, 954 bp, synthetic construct
  misc_feature 1..954: note = Magnetococcus marinus, codon optimized, with N-terminal 3x HA tag and linker:
  source 1..954: mol_type = other DNA
atggtttatccgtatgatgttcctgactatgcaggatcctatccttatgatgttcccgattacgctggttcttacccttacgatgttcccgattatgccagttctggattggtgccacgaggcagccacatgagccggaagatcagagatcttatcgaatctaagagatttcagaatgttattaccgcgataatcgtactcaacggggcggtgctcggtctcctcaccgataccacattgagcgcttctagccagaacctgctcgaaagggttgaccaactgtgcctgacaatttttatcgtggaaattagcttgaaaatttacgcctacggcgttcgcggttttttccggagcggttggaatctttttgacttcgttatcgttgccatcgcgctcatgcccgcacagggttctttgtctgtgttgaggacattccgaatatttcgcgtgatgcgcttggtatccgtgatccctacgatgcgccgcgtcgtacaaggaatgttgctggctctccccggcgtcgggagcgttgctgccctccttaccgtggtattttacatagcggcggttatggctactaatctttacggagctaccttcccggagtggttcggggatttgtccaagagcctctatacattgtttcaagttatgaccctggagtcctggtctatgggcattgtccggcccgtaatgaacgtacacccaaatgcgtgggtgtttttcattccattcatcatgctgactacctttaccgtgctgaacttgttcattgggattatcgtggatgcgatggccatcactaaggagcaagaagaagaggctaaaactggccaccaccaagagccaatttctcaaaccctcttgcatctcggggaccgactggaccgcattgagaagcaactcgcgcagaacaatgagctgttgcagcgacagcaacctcaaaaaaaataa

SEQ ID NO: 19 - DNA, 885 bp, synthetic construct
  misc_feature 1..885: note = Magnetococcus marinus
  source 1..885: mol_type = other DNA
atgggcagcagccatcatcatcatcatcacagcagcggcctggtgccgcgcggcagccatatgtcacgcaaaatccgcgatttaatcgaatccaaacgctttcaaaacgtcatcaccgccattattgtgctcaatggcgctgtgctgggtctgctgaccgatacaaccctgtcggcctccagccaaaacctgctggagcgtgtggatcaactttgtctgactatctttattgttgaaatctccctgaaaatctacgcctatggcgtgcgcggctttttccgcagcggctggaatctgtttgattttgtgattgtggccatcgcgcttatgccggcccagggtagcctgtcggtgctgcgtaccttccgtatcttccgcgtcatgcgcctcgtatcggtcatcccaaccatgcgccgtgtggtgcaaggcatgctcttggcactgccgggcgtgggctcggtagcggcactgttgacggtggtcttctatattgcggctgtcatggccaccaatctctacggggcaaccttccctgaatggtttggtgatcttagcaagagcctgtacacactgtttcaggtgatgaccttagagtcatggtctatgggcattgtgcgtccagtgatgaacgttcatccgaacgcatgggtttttttcatcccgttcatcatgctcaccacctttaccgtgctcaacctgtttattggcattattgtagatgcaatggcaatcaccaaggaacaggaggaagaggccaaaaccggtcaccatcaagaacctatttctcaaactcttcttcatcttggtgatcgtcttgatcgtattgaaaaacaacttgctcaaaataatgaacttcttcaacgtcaacaacctcaaaaaaaataa

SEQ ID NO: 20 - DNA, 855 bp, Bacillus pseudofirmus
  source 1..855: mol_type = genomic DNA
atggaaaacaatccagccgaacaacaagttccaccattagtagccttagctcagcgtatcgtctttcataaggcctttaccccaactattattaccttgattatcattaatgccattattgtaggccttgaaacatatcctactgtttatcaaggttataatgattggttctacgcagcagatttagccttactttggatttttacaattgagattacactgcgttttatcgcagcgagaccgactaaatctttttttaaaagcagctggaactggtttgatttattaatcgttcttgccggtcatgtctttgccggtgctcattttgtaacggttcttcgtatcctgcgcgttcttcgcgtattacgtgccatttctgtcattccttctctgcgtcgtttagtcgatgctttgctgatgaccatcccggctttaggaaacattatgatcctgatgggaattattttctatattttcgctgtgattggaacgatgttatttgcttctgtagcacctgagtactttggtaacttacagctttctttattaacattattccaagttgttacacttgaatcttgggcaagcggtgtcatgaggccgatttttgcagaggtttggtggtcttggatttattttgtcatctttattttagtagggacatttattgtctttaacttatttatcggtgttatcgttaataacgttgaaaaagcaaacgaagaagaactcaaatcagaattagatgataaagaggcagatacaaaagaagagcttgcttctctgcgtaatgaagtagcagagatgaaagacctcattaaacaaatgcataaacagcaaacaaaaaaagggtaa

SEQ ID NO: 21 - DNA, 951 bp, synthetic construct
  misc_feature 1..951: note = Bacillus pseudofirmus, codon optimized, with N-terminal 3x HA tag
  source 1..951: mol_type = other DNA
atggtttacccgtatgatgtcccggattacgctggcagctacccatacgatgtacccgactatgccggcagttatccctacgacgtccctgactacgcagaaaacaacccagccgaacagcaagtcccacccctcgtggcgctcgcccaacgcatagtatttcacaaggcgtttacgccgacgataatcaccctcatcattattaatgcgatcattgtgggactcgagacatacccaacggtttaccagggttacaatgattggttctatgctgccgaccttgctttgttgtggatattcactattgaaatcacgctccgattcatcgccgcccgaccgacgaagagtttcttcaagtctagctggaactggtttgatctgcttatcgtattggcgggccacgtcttcgctggcgcccattttgttacggtgcttaggatcctccgcgtcctgagggtcctcagagctatctcagtcatacccagtctccggcggctggttgacgcacttttgatgacaatcccagcactcggtaacatcatgatactgatggggattattttttacatattcgcggttatcgggacgatgctctttgcatcagtagcgccagaatactttggcaatttgcagctgtctctgcttacactgttccaagtggttacgctggaaagttgggctagtggggttatgcgacctatttttgccgaagtctggtggtcttggatctattttgtaatctttattctcgtgggaactttcatagtatttaaccttttcattggcgtcatcgtgaacaatgtggaaaaagctaacgaagaggaactgaaaagcgaactggatgataaagaggctgatacaaaagaagaactggcatcattgcgaaacgaggtggcagaaatgaaggatctcataaaacagatgcataaacagcaaacaaaaaagggttaa

SEQ ID NO: 22 - DNA, 984 bp, synthetic construct
  misc_feature 1..984: note = Shewanella putrifaciens-D60N, codon optimized, with N-terminal 3x HA tag
  source 1..984: mol_type = other DNA
atggtttacccgtatgatgtcccggattacgctggcagctacccatacgatgtacccgactatgccggcagttatccctacgacgtccctgactacgcatctacgtcccttttgaatgcgcctaccggccttcaagctagagtcattaatctcgtcgaacaaaactggtttggacactttatactgactctcatactcattaatgctgtgcagcttggaatggaaactagcgccagcctcatggcacaatatggcgcgctgcttatgtccttgaataaggtccttctctctgtgttcgtggtcgaactgctgctccggatttatgcgtatcggggcaagttttttaaggacccgtggaatgtgtttgacttcactgttattgttattgctctgattcctgcatctggcccattggctgtcctccgctccctccgagttctccgcgtcttgagggttctgacgattgtccccagcatgaaaagagtagtgtcagcactgcttgggagcttgcccgggttggcctccattgcaaccgtgcttctgttgatctattacgttttcgctgtgatcgccactaaaattttcggggatgcttttccggaatggttcgggacgatagcggactccttctataccctttttcaaattatgaccttggaaagttggtctatggggatctctaggccagtgatggaggtgtacccttacgcttgggtattctttgtgccctttattcttgttgctacttttaccatgcttaaccttttcatcgccatcatagtgaatactatgcagacattctctgacgaggaacatgctctggagcgagagcaagataaacagatcttggaacaggagcagagacaaatgcacgaggaactgaaggccattcgactcgagcttcagcaactccaaacccttttgcgaaatgcggctggggactcctccaatgtctccacaaagggcaatatcggctcagactaa

SEQ ID NO: 23 - DNA, 888 bp, Shewanella putrifaciens
  source 1..888: mol_type = genomic DNA
atgagtacatctttacttaacgcgccaacgggtttgcaggcacgagtgattaacttggttgagcaaaactggtttggtcattttattttgacattgattttaatcaacgcggtgcagttaggtatggagacctcagccagcctgatggcgcaatacggtgctttgttgatgagtcttgataaggtgctgctgagtgtatttgtggtggagttattgctgcggatttatgcctacagggggaaattttttaaagacccttggaacgtgttcgattttaccgtgatagtgatagcactgatccctgcatctgggccattggctgtcctgcgttcgctcagggtattgcgggtgctgagagtgttaacaattgtgccatcaatgaaacgggtggtgtctgcgctgttgggatcacttcctggattggcatcgatcgccacagtattactgctgatttattatgtgtttgcggtgatcgctaccaaaatttttggcgatgcattccctgaatggtttggcactattgctgactcattttataccctatttcaaataatgacgcttgaaagctggtctatgggaatttcgcggccagtgatggaagtctacccttatgcttgggtatttttcgtaccatttattctggtagcgactttcacaatgctaaatttgtttattgcgattatcgtcaataccatgcaaaccttcagcgacgaagagcatgcattagagcgtgagcaagacaaacaaatcttagagcaggaacaaagacaaatgcacgaggagttgaaagccatcagactcgagctacaacaattacaaaccttgctgcgcaatgctgctggtgattcttctaatgtgtcgacaaagggaaacattggttctgactaa

SEQ ID NO: 24 - DNA, 246 bp, synthetic construct
  misc_feature 1..246: note = WPRE3
  source 1..246: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgg

SEQ ID NO: 25 - DNA, 204 bp, synthetic construct
  misc_feature 1..204: note = BGHpA
  source 1..204: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 26 - DNA, 78 bp, synthetic construct
  misc_feature 1..78: note = P2A
  source 1..78: mol_type = other DNA
ggcagcggcgccaccaacttcagcctgctgaagcaggccggcgacgtggaggagaaccccggccccggagctagcgga

SEQ ID NO: 27 - protein, 26 aa, synthetic construct
  REGION 1..26: note = P2A
  REGION 1..3: note = MISC_FEATURE - (GSG) residues can be added to the 5' end of the peptide to improve cleavage efficiency
GSGATNFSLLKQAGDVEENPGPGASG

SEQ ID NO: 28 - protein, 21 aa, synthetic construct
  REGION 1..21: note = T2A
  REGION 1..3: note = MISC_FEATURE - (GSG) residues can be added to the 5' end of the peptide to improve cleavage efficiency
GSGEGRGSLLTCGDVEENPGP

SEQ ID NO: 29 - protein, 24 aa, synthetic construct
  REGION 1..24: note = E2A
  REGION 1..3: note = MISC_FEATURE - (GSG) residues can be added to the 5' end of the peptide to improve cleavage efficiency
GSGQCTNYALLKLAGDVESNPGPP

SEQ ID NO: 30 - protein, 25 aa, synthetic construct
  REGION 1..25: note = F2A
  REGION 1..3: note = MISC_FEATURE - (GSG) residues can be added to the 5' end of the peptide to improve cleavage efficiency
GSGVKQTLNFDLLKLAGDVESNPGP

SEQ ID NO: 31 - DNA, 99 bp, synthetic construct
  misc_feature 1..99: note = N-terminal 3XHA tag
  source 1..99: mol_type = other DNA
atggtttacccgtatgatgtcccggattacgctggcagctacccatacgatgtacccgactatgccggcagttatccctacgacgtccctgactacgca

SEQ ID NO: 32 - protein, 33 aa, synthetic construct
  REGION 1..33: note = N-terminal 3XHA tag
MVYPYDVPDYAGSYPYDVPDYAGSYPYDVPDYA

SEQ ID NO: 33 - protein, 11 aa, synthetic construct
  REGION 1..11: note = change from AAV9 to PHP.eB capsid
SDGTLAVPFKA

SEQ ID NO: 34 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - DNA, 747 bp, synthetic construct
  misc_feature 1..747: note = tet-Transactivator version 2 (tTA2)
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 36 - DNA, 2843 bp, synthetic construct
  misc_feature 1..2843: note = rAAV: hI56i-minBglobin-His-NavMs-P2A-SYFP2-WPRE3-BGHpA
  source 1..2843: mol_type = other DNA
gcggccgcacgcgtataggtaccgagctctatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagggagctcgggctgggcataaaagtcagggcagagccatctattgcttacatttgcttctgggatccagatctttcgaagctagcgctaccggtcgccaccatgggcagcagccatcatcatcatcatcacagcagcggcctggtgccgcgcggcagccatatgtcacgcaaaatccgcgatttaatcgaatccaaacgctttcaaaacgtcatcaccgccattattgtgctcaatggcgctgtgctgggtctgctgaccgatacaaccctgtcggcctccagccaaaacctgctggagcgtgtggatcaactttgtctgactatctttattgttgaaatctccctgaaaatctacgcctatggcgtgcgcggctttttccgcagcggctggaatctgtttgattttgtgattgtggccatcgcgcttatgccggcccagggtagcctgtcggtgctgcgtaccttccgtatcttccgcgtcatgcgcctcgtatcggtcatcccaaccatgcgccgtgtggtgcaaggcatgctcttggcactgccgggcgtgggctcggtagcggcactgttgacggtggtcttctatattgcggctgtcatggccaccaatctctacggggcaaccttccctgaatggtttggtgatcttagcaagagcctgtacacactgtttcaggtgatgaccttagagtcatggtctatgggcattgtgcgtccagtgatgaacgttcatccgaacgcatgggtttttttcatcccgttcatcatgctcaccacctttaccgtgctcaacctgtttattggcattattgtagatgcaatggcaatcaccaaggaacaggaggaagaggccaaaaccggtcaccatcaagaacctatttctcaaactcttcttcatcttggtgatcgtcttgatcgtattgaaaaacaacttgctcaaaataatgaacttcttcaacgtcaacaacctcaaaaaaaaggcagcggcgccaccaacttcagcctgctgaagcaggccggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 37 - DNA, 2835 bp, synthetic construct
  misc_feature 1..2835: note = rAAV:3xhI56iCore-minCMV-SYFP2-P2A-3xHA-NavSheP-D60N -WPRE3-BGHpA
  source 1..2835: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtcgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggagctagcggaatggtttacccgtatgatgtcccggattacgctggcagctacccatacgatgtacccgactatgccggcagttatccctacgacgtccctgactacgcatctacgtcccttttgaatgcgcctaccggccttcaagctagagtcattaatctcgtcgaacaaaactggtttggacactttatactgactctcatactcattaatgctgtgcagcttggaatggaaactagcgccagcctcatggcacaatatggcgcgctgcttatgtccttgaataaggtccttctctctgtgttcgtggtcgaactgctgctccggatttatgcgtatcggggcaagttttttaaggacccgtggaatgtgtttgacttcactgttattgttattgctctgattcctgcatctggcccattggctgtcctccgctccctccgagttctccgcgtcttgagggttctgacgattgtccccagcatgaaaagagtagtgtcagcactgcttgggagcttgcccgggttggcctccattgcaaccgtgcttctgttgatctattacgttttcgctgtgatcgccactaaaattttcggggatgcttttccggaatggttcgggacgatagcggactccttctataccctttttcaaattatgaccttggaaagttggtctatggggatctctaggccagtgatggaggtgtacccttacgcttgggtattctttgtgccctttattcttgttgctacttttaccatgcttaaccttttcatcgccatcatagtgaatactatgcagacattctctgacgaggaacatgctctggagcgagagcaagataaacagatcttggaacaggagcagagacaaatgcacgaggaactgaaggccattcgactcgagcttcagcaactccaaacccttttgcgaaatgcggctggggactcctccaatgtctccacaaagggcaatatcggctcagactaatgaccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 38 - DNA, 2802 bp, synthetic construct
  misc_feature 1..2802: note = rAAV: 3xhI56iCore-minCMV-SYFP2-P2A-3xHA-NavBp-WPRE3-BGHpA
  source 1..2802: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtcgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggagctagcggaatggtttacccgtatgatgtcccggattacgctggcagctacccatacgatgtacccgactatgccggcagttatccctacgacgtccctgactacgcagaaaacaacccagccgaacagcaagtcccacccctcgtggcgctcgcccaacgcatagtatttcacaaggcgtttacgccgacgataatcaccctcatcattattaatgcgatcattgtgggactcgagacatacccaacggtttaccagggttacaatgattggttctatgctgccgaccttgctttgttgtggatattcactattgaaatcacgctccgattcatcgccgcccgaccgacgaagagtttcttcaagtctagctggaactggtttgatctgcttatcgtattggcgggccacgtcttcgctggcgcccattttgttacggtgcttaggatcctccgcgtcctgagggtcctcagagctatctcagtcatacccagtctccggcggctggttgacgcacttttgatgacaatcccagcactcggtaacatcatgatactgatggggattattttttacatattcgcggttatcgggacgatgctctttgcatcagtagcgccagaatactttggcaatttgcagctgtctctgcttacactgttccaagtggttacgctggaaagttgggctagtggggttatgcgacctatttttgccgaagtctggtggtcttggatctattttgtaatctttattctcgtgggaactttcatagtatttaaccttttcattggcgtcatcgtgaacaatgtggaaaaagctaacgaagaggaactgaaaagcgaactggatgataaagaggctgatacaaaagaagaactggcatcattgcgaaacgaggtggcagaaatgaaggatctcataaaacagatgcataaacagcaaacaaaaaagggttaatgaccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 39 - DNA, 2805 bp, synthetic construct
  misc_feature 1..2805: note = rAAV: 3xhI56iCore-minCMV-SYFP2-P2A-3xHA-NavMs-WPRE3-BGHpA
  source 1..2805: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtcgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggagctagcggaatggtttatccgtatgatgttcctgactatgcaggatcctatccttatgatgttcccgattacgctggttcttacccttacgatgttcccgattatgccagttctggattggtgccacgaggcagccacatgagccggaagatcagagatcttatcgaatctaagagatttcagaatgttattaccgcgataatcgtactcaacggggcggtgctcggtctcctcaccgataccacattgagcgcttctagccagaacctgctcgaaagggttgaccaactgtgcctgacaatttttatcgtggaaattagcttgaaaatttacgcctacggcgttcgcggttttttccggagcggttggaatctttttgacttcgttatcgttgccatcgcgctcatgcccgcacagggttctttgtctgtgttgaggacattccgaatatttcgcgtgatgcgcttggtatccgtgatccctacgatgcgccgcgtcgtacaaggaatgttgctggctctccccggcgtcgggagcgttgctgccctccttaccgtggtattttacatagcggcggttatggctactaatctttacggagctaccttcccggagtggttcggggatttgtccaagagcctctatacattgtttcaagttatgaccctggagtcctggtctatgggcattgtccggcccgtaatgaacgtacacccaaatgcgtgggtgtttttcattccattcatcatgctgactacctttaccgtgctgaacttgttcattgggattatcgtggatgcgatggccatcactaaggagcaagaagaagaggctaaaactggccaccaccaagagccaatttctcaaaccctcttgcatctcggggaccgactggaccgcattgagaagcaactcgcgcagaacaatgagctgttgcagcgacagcaacctcaaaaaaaataatgaccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 40 - DNA, 1901 bp, synthetic construct
  misc_feature 1..1901: note = rAAV: hI56i-minBglobin-SYFP2-WPRE3-BGHpA
  source 1..1901: mol_type = other DNA
gcggccgcacgcgtataggtaccgagctctatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 41 - DNA, 4398 bp, synthetic construct
  misc_feature 1..4398: note = rAAV: hI56core-minBglobin-SYFP2-WPRE3-BGHpA
  source 1..4398: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggacctgcagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 42 - DNA, 4635 bp, synthetic construct
  misc_feature 1..4635: note = rAAV: 3XhI56core-minBglobin-SYFP2-WPRE3-BGHpA
  source 1..4635: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - DNA, 4841 bp, synthetic construct
  misc_feature 1..4841: note = rAAV: hI56i-minBglobin-SYFP2-WPRE3-BGHpA
  source 1..4841: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtataggtaccgagctctatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 44 - protein, 1927 aa, Homo sapiens [sequence omitted]

SEQ ID NO: 45 - protein, 465 aa, Homo sapiens
MLPLWTLSLLLGAVAGKEVCYERLGCFSDDSPWSGITERPLHILPWSPKDVNTRFLLYTNENPNNFQEVAADSSSISGSNFKTNRKTRFIIHGFIDKGEENWLANVCKNLFKVESVNCICVDWKGGSRTGYTQASQNIRIVGAEVAYFVEFLQSAFGYSPSNVHVIGHSLGAHAAGEAGRRTNGTIGRITGLDPAEPCFQGTPELVRLDPSDAKFVDVIHTDGAPIVPNLGFGMSQVVGHLDFFPNGGVEMPGCKKNILSQIVDIDGIWEGTRDFAACNHLRSYKYYTDSIVNPDGFAGFPCASYNVFTANKCFPCPSGGCPQMGHYADRYPGKTNDVGQKFYLDTGDASNFARWRYKVSVTLSGKKVTGHILVSLFGNKGNSKQYEIFKGTLKPDSTHSNEFDSDVDVGDLQMVKFIWYNNVINPTLPRVGASKIIVETNVGKQFNFCSPETVREEVLLTLTPC

SEQ ID NO: 46 - protein, 824 aa, Homo sapiens
MSISSLFGGRYDNKFLLNMSSAPKIELIVDKVASLSEGRLEGRLPEDWFRHIMDPETEFNSEFADALCIGIDEFAQPLPFLPFKALLVTGTAGAGKTNSIQTLAANLDCIVTATTSIAAQNLSVVLNRSKSAQVKTIFKTFGFNSSHVSMSERQSYIANDERSIQIQQKQDLSIYWNVISDIADRALGAVACKTKELPDLCESSVIVIDEAGVILRHILHTVVFFYWFYNALYKTPLYEDGIVPCIVCVGSPTQSNALVTSFNPLTQNKDVKRGIDVLSALICDDVLSKYCEVDNNWIIFVNNKRCADHAFGDFLKHIEFGLPLKPELIEYVDQFVKPASYIRNPMNEIETTRLFLSHNEVKNYFRSLHEQVEVTNRNNLFVFPVYFLIKNKTFEDYKSEIGNFSLEIEPWFKSNIHRLNTYSQFADQDLSKTVQLEEIVLEDGSVEETLITCHLKHIRNSSIGVTSKIKASTVGFSGTYEKFVELLQSDLFIEKTSCDQTIHAYSFLSGLMFGGMYSFCCSKFTTPEVLMEIKNIKMPSIEFLESEMSRMSPDVQTVETDERYDFGLVDDGLSDVDLLEIDPCGDPFFTRYSKLPLTNSLSFEEISLLYTTFKDIFISRFAILQKHTKGKFGKTLLVTYNRNNVSRKQCGEIYSHLKSFYGMLTYAIPANNYTLEGYTNDNVVHLGTDKQLPQILYKKGLPRLVIKDEMGFISVLDNNVSKFIDVVNGQSFHLCTTVDYATVSKVSMTITKSQGLSIQKVAIDFGSDPKNLKLSSIYVGMSRVTDPNNLIMNVNPLRLNYENDNFIAPHIVKALKNENTMLIF

SEQ ID NO: 47 - protein, 511 aa, Homo sapiens
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 48 - protein, 952 aa, Homo sapiens [sequence omitted]

SEQ ID NO: 49 - protein, 785 aa, Homo sapiens
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 50 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 51 - protein, 529 aa, Homo sapiens
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 52 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 53 - protein, 743 aa, Homo sapiens
MNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSASQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 54 - protein, 507 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 55 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 56 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 57 - protein, 1338 aa, Homo sapiens [sequence omitted]

SEQ ID NO: 58 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 59 - protein, 178 aa, Homo sapiens [sequence omitted]

SEQ ID NO: 60 - protein, 866 aa, Homo sapiens
MGNRGMEELIPLVNKLQDAFSSIGQSCHLDLPQIAVVGGQSAGKSSVLENFVGRDFLPRGSGIVTRRPLILQLIFSKTEHAEFLHCKSKKFTDFDEVRQEIEAETDRVTGTNKGISPVPINLRVYSPHVLNLTLIDLPGITKVPVGDQPPDIEYRVKDMILQFISRESSLILAVTPANMDLANSDALKLAKEVDPQGLRTIGVITKLDLMDEGTDARDVLENKLLPLRRGYIGVVNRSQKDIEGKKDIRAALAAERKFFLSHPAYRHMADRMGTPHLQKTLNQQLTNHIRESLPALRSKLQSQLLSLEKEVEEYKIFRPDDPTPKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELINTVRQCTSKLSSYPRLREETERIVTTYIREREGRTKDQILLLIDIEQSYINTNHEDFIGFANAQQRSTQLNKKRAIPNQVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESADQAQRRDDMLRMYHALKEALNIIGDISTSTVSTPVPPPVDDTWLQSASSHSPTPQRRPVSSIHPPGRPPAVRGPTPGPPLIPVPVGAAASFSAPPIPSRPGPQSVFANSDLFPAPPQIPSRPVRIPPGIPPGVPSRRPPAAPSRPTIIRPAEPSLLD

SEQ ID NO: 61 - protein, 1675 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 62 - protein, 1640 aa, Homo sapiens
MAQILPVRFQEHFQLQNLGINPANIGFSTLTMESDKFICIREKVGEQAQVTIIDMSDPMAPIRRPISAESAIMNPASKVIALKAGKTLQIFNIEMKSKMKAHTMAEEVIFWKWVSVNTVALVTETAVYHWSMEGDSQPMKMFDRHTSLVGCQVIHYRTDEYQKWLLLVGISAQQNRVVGAMQLYSVDRKVSQPIEGHAAAFAEFKMEGNAKPATLFCFAVRNPTGGKLHIIEVGQPAAGNQPFVKKAVDVFFPPEAQNDFPVAMQIGAKHGVIYLITKYGYLHLYDLESGVCICMNRISADTIFVTAPHKPTSGIIGVNKKGQVLSVCVEEDNIVNYATNVLQNPDLGLRLAVRSNLAGAEKLFVRKFNTLFAQGSYAEAAKVAASAPKGILRTRETVQKFQSIPAQSGQASPLLQYFGILLDQGQLNKLESLELCHLVLQQGRKQLLEKWLKEDKLECSEELGDLVKTTDPMLALSVYLRANVPSKVIQCFAETGQFQKIVLYAKKVGYTPDWIFLLRGVMKISPEQGLQFSRMLVQDEEPLANISQIVDIFMENSLIQQCTSFLLDALKNNRPAEGLLQTWLLEMNLVHAPQVADAILGNKMFTHYDRAHIAQLCEKAGLLQQALEHYTDLYDIKRAVVHTHLLNPEWLVNFFGSLSVEDSVECLHAMLSANIRQNLQLCVQVASKYHEQLGTQALVELFESFKSYKGLFYFLGSIVNFSQDPDVHLKYIQAACKTGQIKEVERICRESSCYNPERVKNFLKEAKLTDQLPLIIVCDRFGFVHDLVLYLYRNNLQRYIEIYVQKVNPSRTPAVIGGLLDVDCSEEVIKHLIMAVRGQFSTDELVAEVEKRNRLKLLLPWLESQIQEGCEEPATHNALAKIYIDSNNSPECFLRENAYYDSSVVGRYCEKRDPHLACVAYERGQCDLELIKVCNENSLFKSEARYLVCRKDPELWAHVLEETNPSRRQLIDQVVQTALSETRDPEEISVTVKAFMTADLPNELIELLEKIVLDNSVFSEHRNLQNLLILTAIKADRTRVMEYISRLDNYDALDIASIAVSSALYEEAFTVFHKFDMNASAIQVLIEHIGNLDRAYEFAERCNEPAVWSQLAQAQLQKDLVKEAINSYIRGDDPSSYLEVVQSASRSNNWEDLVKFLQMARKKGRESYIETELIFALAKTSRVSELEDFINGPNNAHIQQVGDRCYEEGMYEAAKLLYSNVSNFARLASTLVHLGEYQAAVDNSRKASSTRTWKEVCFACMDGQEFRFAQLCGLHIVIHADELEELMCYYQDRGYFEELILLLEAALGLERAHMGMFTELAILYSKFKPQKMLEHLELFWSRVNIPKVLRAAEQAHLWAELVFLYDKYEEYDNAVLTMMSHPTEAWKEGQFKDIITKVANVELCYRALQFYLDYKPLLINDLLLVLSPRLDHTWTVSFFSKAGQLPLVKPYLRSVQSHNNKSVNEALNHLLTEEEDYQGLRASIDAYDNFDNISLAQQLEKHQLMEFRCIAAYLYKGNNWWAQSVELCKKDHLYKDAMQHAAESRDAELAQKLLQWFLEEGKRECFAACLFTCYDLLRPDMVLELAWRHNLVDLAMPYFIQVMREYLSKVDKLDALESLRKQEEHVTEPAPLVFDFDGHE

SEQ ID NO: 63 - protein, 218 aa, Homo sapiens
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRAAEEAFVNDIDESSPGTEWERVARLCDFNPKSSKQAKDVSRMRSVLISLKQAPLVH

SEQ ID NO: 64 - protein, 211 aa, Homo sapiens
MADDFGFFSSSESGAPEAAEEDPAAAFLAQQESEIAGIENDEGFGAPAGSHAAPAQPGPTSGAGSEDMGTTVNGDVFQEANGPADGYAAIAQADRLTQEPESIRKWREEQRKRLQELDAASKVTEQEWREKAKKDLEEWNQRQSEQVEKNKINNRASEEAFVKESKEETPGTEWEKVAQLCDFNPKSSKQCKDVSRLRSVLMSLKQTPLSR

SEQ ID NO: 65 - protein, 218 aa, Homo sapiens
MSQTAMSETYDFLFKFLVIGNAGTGKSCLLHQFIEKKFKDDSNHTIGVEFGSKIINVGGKYVKLQIWDTAGQERFRSVTRSYYRGAAGALLVYDITSRETYNALTNWLTDARMLASQNIVIILCGNKKDLDADREVTFLEASRFAQENELMFLETSALTGENVEEAFVQCARKILNKIESGELDPERMGSGIQYGDAALRQLRSPRRAQAPNAQECGC

SEQ ID NO: 66 - protein, 216 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 67 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 68 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 69 - protein, 257 aa, Homo sapiens
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 70 - protein, 71 aa, Homo sapiens
MRPSGTAGAALLALLAALCPASRALEEKKGKGVSRRLPRRPRIAPRTPQPAQPRTGAPARARAPARPFLFP

SEQ ID NO: 71 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 72 - protein, 41 aa, Homo sapiens
YGQVPMCDAGEQCAVRKGARIGKLCDCPRGTSCNSFLLKCL

SEQ ID NO: 73 - protein, 129 aa, Homo sapiens
MKILVALAVFFLVSTQLFAEEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 74 - protein, 11 aa, Homo sapiens
RPKPQQFFGLM

SEQ ID NO: 75 - protein, 125 aa, Homo sapiens
MAGPSLACCLLGLLALTSACYIQNCPLGGKRAAPDLDVRKCLPCGPGGKGRCFGPNICCAEELGCFVGTAEALRCQEENYLPSPCQSGQKACGSGGRCAVLGLCCSPDGCHADPACDAEATFSQR

SEQ ID NO: 76 - protein, 9 aa, Homo sapiens
CYIQNCPLG

SEQ ID NO: 77 - protein, 116 aa, Homo sapiens
MLSCRLQCALAALSIVLALGCVTGAPSDPRLRQFLQKSLAAAAGKQELAKYFLAELLSEPNQTENDALEPEDLSQAAEQDEMRLELQRSANSNPAMAPRERKAGCKNFFWKTFTSC

SEQ ID NO: 78 - protein, 449 aa, synthetic construct
  REGION 1..449: note = synthetic construct of Myosin light chain kinase, Green fluorescent protein, Calmodulin chimera
  SITE 223: note = misc_feature - Xaa can be any naturally occurring amino acid
MRGSHHHHHHGMASMTGGQQMGRDLYDDDDKDLATMVDSSRRKWNKTGHAVRAIGRLSSLENVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLXVQCFSRYPDHMKQHDFFKSAMPEGYIQERTIFFKDDGNYKTRAEVKFEGDTLVNRIELKGIDFKEDGNILGHKLEYNTRDQLTEEQIAEFKEAFSLFDKDGDGGITTKQLGTVMRSLGQNPTEAELQDMINEVGADGNGTIDFPQFLTMMARKMKDTDSEEEIREAFRVFGKDGNGYISAAQLRHVMTNLGEKLTDEEVDEMIREAGIDGDGQVNYEQFVQMMTAK

SEQ ID NO: 79 - protein, 309 aa, synthetic construct
  REGION 1..309: note = synthetic construct of Genetically-encoded green calcium indicator NTnC
  SITE 68: note = misc_feature - Xaa can be any naturally occurring amino acid
MVSKGEEDNMASLPATHELHIFGSINGVDFDMVGQGSGNPNVGYEELNLKSTKGDLQFSPWILVPHIXFHQYLPYPDGMSPFQAAMVDGSGYQVHRTVQFEDGASLTVNYRYTYEGSHIKGEAQVKGTGFPADGPVMANSLTAMVPSEEELSECFRTFDKDGDGFIDREEFGGIIRLTGEQLTDEDPDEIFGDSDTDKNGRIDFDEFLKMVENVQLSMADWCRSKMACPNDKTLISTLKWSYTTGNGKRYRSTARTTYTFAKPMAANYLKNQPMYVFRKTELKHSKTELNFKEWQKAFTDVMGMDELYK

SEQ ID NO: 80 - protein, 619 aa, synthetic construct
  REGION 1..619: note = synthetic construct of Calcium indicator TN-XXL
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELANCFRIFDKDANGFIDIEELGEILRATGEHVTEEDIEDLMKDSDKNNDGRIDFDEFLKMMEGVQGTSEEELANCFRIFDKDANGFIDIEELGEILRATGEHVTEEDIEDLMKDSDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 81 - protein, 730 aa, synthetic construct
  REGION 1..730: note = synthetic construct of BRET-based auto-luminescent calcium indicator
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKRMHDQLTEEQIAEFKEAFSLFDKDGDGTITTKELGTVMRSLGQNPTEAELQDMINEVDADGNGTIYFPEFLTMMARKMKDTDSEEEIREAFRVFDKDGNGYISAAQLRHVMTNLGEKLTDEEVDEMIREADIDGDGQVNYEEFVQMMTAKGGKRRWKKNFIAVSAANRFKKISSSGALELMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 82 - protein, 568 aa, synthetic construct
  REGION 1..568: note = synthetic construct of Calcium indicator protein OeNL(Ca2+)-18u
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITEQVEDAVAHSGTLEDFVGDWRQTAGYNLDQVLEQGGVSSLFQNLGVSVTPIQRIVLSGENGLKIDIHVIIPYEGPWMHDQLTEEQIAEFKEAFSLFDKDGDGTITTKELGTVMRSLGQNPTEAELQDMINEVDADGNGTIYFPDFLTMMARKMKDTDSEEEIREAFRVFDKDGNGYISAADLRHVMTNLGEKLTDEEVDEMIREADIDGEGQVNYEEFVQMMTAKGGKRRWKKNFIAVSAANRFKKISSSGALELLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 83 - protein, 593 aa, Mesostigma viride
MSPPTSPTPDTGHDTPDTGHDTGGHGAVEICFAPCEEDCVTIRYFVENDFEGCIPGHFDQYSSHGSLHDIVKAALYICMVISILQILFYGFQWWRKTCGWEVWFVACIETSIYIIAITSEADSPFTLYLTNGQISPQLRYMEWLMTCPVILIALSNITGMAEEYNKRTMTLLTSDVCCIVLGMMSAASKPRLKGILYAVGWAFGAWTYWTALQVYRDAHKAVPKPLAWYVRAMGYVFFTSWLTFPGWFLLGPEGLEVVTGTVSTLMHACSDLISKNLWGFMDWHLRVLVARHHRKLFKAEEEHALKKGQTLEPGMPRSTSFVRGLGDDVEIDPSYELYRLKRQNHPEYFLSPAQTPRRGPSFDKRTSFEMDGGKNGMLQMMPVTGMGMGMGMGMGGGKTVLFLDYTGGGYVSFFEQQLSNMGVNVTKCWSDDDMYNTAGVANVKQLFHFAMIPNNALGGQMVMDLRGTGLLVVAYGPEPPMPGMGQDEFVPLQMPGVPYDESILHNLVMRHAITQGLGMNGMQGNMGQQQQMMGMQGNMNGMQGNMNGMQGNMNGMQGNMSGMQGNMNGMQGNSGMNQGWNNQGFTNTGAFGY

SEQ ID NO: 84 - protein, 747 aa, Volvox carteri
MDHPVARSLIGSSYTNLNNGSIVIPSDACFCMKWLKSKGSPVALKMANALQWAAFALSVIILIYYAYATWRTTCGWEEVYVCCVELTKVVIEFFHEFDEPGMLYLANGNRVLWLRYGEWLLTCPVILIHLSNLTGLKDDYNKRTMRLLVSDVGTIVWGATAAMSTGYIKVIFFLLGCMYGANTFFHAAKVYIESYHTVPKGLCRQLVRAMAWLFFVSWGMFPVLFLLGPEGFGHLSVYGSTIGHTIIDLLSKNCWGLLGHFLRLKIHEHILLYGDIRKVQKIRVAGEELEVETLMTEEAPDTVKKSTAQYANRESFLTMRDKLKEKGFEVRASLDNSGIDAVINHNNNYNNALANAAAAVGKPGMELSKLDHVAANAAGMGGIADHVATTSGAISPGRVILAVPDISMVDYFREQFAQLPVQYEVVPALGADNAVQLVVQAAGLGGCDFVLLHPEFLRDKSSTSLPARLRSIGQRVAAFGWSPVGPVRDLIESAGLDGWLEGPSFGLGISLPNLASLVLRMQHARKMAAMLGGMGGMLGSNLMSGSGGVGLMGAGSPGGGGGAMGVGMTGMGMVGTNAMGRGAVGNSVANASMGGGSAGMGMGMMGMVGAGVGGQQQMGANGMGPTSFQLGSNPLYNTAPSPLSSQPGGDASAAAAAAAAAAATGAASNSMNAMQAGGSVRNSGILAGGLGSMMGPPGAPAAPTAAATAAPAVTMGAPGGGGAAASEAEMLQQLMAEINRLKSELGE

SEQ ID NO: 85 - protein, 282 aa, Streptococcus pyogenes
MLEHKIDFMVTLEVKEANANGDPLNGNMPRTDAKGYGVMSDVSIKRKIRNRLQDMGKSIFVQANERIEDDFRSLEKRFSQHFTAKTPDKEIEEKANALWFDVRAFGQVFTYLKKSIGVRGPVSISMAKSLEPIVISSLQITRSTNGMEAKNNSGRSSDTMGTKHFVDYGVYVLKGSINAYFAEKTGFSQEDAEAIKEVLVSLFENDASSARPEGSMRVCEVFWFTHSSKLGNVSSARVFDLLEYHQSIEEKSTYDAYQIHLNQEKLAKYEAKGLTLEILEGL

SEQ ID NO: 86 - protein, 1368 aa, synthetic construct [sequence omitted]

SEQ ID NO: 87 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 88 - protein, 741 aa, Homo sapiens
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 89 - protein, 357 aa, Homo sapiens
MMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 90 - protein, 743 aa, synthetic construct
  REGION 1..743: note = PHP.eB capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 91 - protein, 7 aa, synthetic construct
  REGION 1..7: note = AAV-BR1
NRGTEWD

SEQ ID NO: 92 - protein, 7 aa, synthetic construct
  REGION 1..7: note = AAV-PHP.S
QAVRTSL

SEQ ID NO: 93 - protein, 7 aa, synthetic construct
  REGION 1..7: note = AAV-PHP.B
TLAVPFK

SEQ ID NO: 94 - protein, 7 aa, synthetic construct
  REGION 1..7: note = AAV-PPS
DSPAHPS

SEQ ID NO: 95 - DNA, 4398 bp, synthetic construct
  misc_feature 1..4398: note = rAAV: hI56core-minBglobin-SYFP2-WPRE3-BGHpA bottom strand
  source 1..4398: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgtgagatctcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaagatctctcgagccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatgatatcgaattcgcggccgcggcgcgccgtcgacttacttgtacagctcgtccatgccgagagtgatcccggcggcggtcacgaactccagcaggaccatgtgatcgcgcttctcgttggggtctttgctcagcttggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcttcgaaagatctggatcccagaagcaaatgtaagcaatagatggctctgccctgacttttatgcccagcccgagctccctgcaggtccattctcttaaatgcagccataattagagtaatttttcatgtagcccgctgattacagcgtttttaccgtcaaagataattacctgtaattttcttccacttttaatactaaaaagccatctttatttagattcaggaacaggaaaggcgaacgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 96 - DNA, 4635 bp, synthetic construct
  misc_feature 1..4635: note = rAAV: 3XhI56core-minBglobin-SYFP2-WPRE3-BGHpA bottom strand
  source 1..4635: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgtgagatctcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaagatctctcgagccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatgatatcgaattcgcggccgcggcgcgccgtcgacttacttgtacagctcgtccatgccgagagtgatcccggcggcggtcacgaactccagcaggaccatgtgatcgcgcttctcgttggggtctttgctcagcttggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcttcgaaagatctggatcccagaagcaaatgtaagcaatagatggctctgccctgacttttatgcccagcccgagctccattctcttaaatgcagccataattagagtaatttttcatgtagcccgctgattacagcgtttttaccgtcaaagataattacctgtaattttcttccacttttaatactaaaaagccatctttatttagccattctcttaaatgcagccataattagagtaatttttcatgtagcccgctgattacagcgtttttaccgtcaaagataattacctgtaattttcttccacttttaatactaaaaagccatctttatttagccattctcttaaatgcagccataattagagtaatttttcatgtagcccgctgattacagcgtttttaccgtcaaagataattacctgtaattttcttccacttttaatactaaaaagccatctttatttagggtaccacgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 97 - DNA, 4841 bp, synthetic construct
  misc_feature 1..4841: note = rAAV: hI56i-minBglobin-SYFP2-WPRE3-BGHpA bottom strand
  source 1..4841: mol_type = other DNA
ctagatctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtcgacgcatgcgggcccggatctgtccctctctgcgcgctcgctcgctcactgaggccgcccgggcaaagcccgggcgtcgggcgacctttggtcgcccggcctcagtgagcgagcgagcgcgcagagagggagtggactagtcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatgatatcgaattcgcggccgcggcgcgccgtcgacttacttgtacagctcgtccatgccgagagtgatcccggcggcggtcacgaactccagcaggaccatgtgatcgcgcttctcgttggggtctttgctcagcttggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcttcgaaagatctggatcccagaagcaaatgtaagcaatagatggctctgccctgacttttatgcccagcccgagctccctactgtgaaactttgggttcgtagctccccaggatcaattctgaacaaagcctccagctgcagtgccatccaatttgaagcagacattggggacaatttaaggtttttatccacaagaaggtttttttccattctcttaaatgcagccataattagagtaatttttcatgtagcccgctgattacagcgtttttaccgtcaaagataattacctgtaattttcttccacttttaatactaaaaagccatctttatttagattcaggaacaggaaaggcgaaacaaaagagggaaattattctgttattcatacacaaattgcagagacgtaggacctaaaattgaaaattaaccaaaattataatgctgaaaagaatggaagaggctgcagaagtacagctggtagtggggctttgctgaattattcaaattacgttagggagaatgctaccatacatcgaaaccaacactaatttttcgttaaaaaaaattcaccagatgcatgccaaaccactgtgagtgcatagagctcggtacctatacgcgtcctaggtcacgactccacccctccaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggcccagatccccccgggaagcttt

SEQ ID NO: 98 - protein, 4 aa, synthetic construct
  REGION 1..4: note = Sequence changed in AAV9
SAQA

SEQ ID NO: 99 - DNA, 1353 bp, synthetic construct
  misc_feature 1..1353: note = GCaMP6m
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaagggagctacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 100 - DNA, 1353 bp, synthetic construct
  misc_feature 1..1353: note = GCaMP6s
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 101 - DNA, 1353 bp, synthetic construct
  misc_feature 1..1353: note = GCaMP6f
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 102 - protein, 717 aa, Chlamydomonas yellowstonensis
MDTLAWVARELLSSGHGTDTATDSGHGTDTSGGHDSSHDAVAHNVTLLIAPPHAGGHAGPTDTSQQITGIDGWIAIPAGDCYCAGWYVSHGSSFEATFAHVCQWSIFAVCVLSLLWYAYQYWKATCGWEEVYVCCIELVFICFELYHEFDSPCSLYLSTSNVVNWLRYSEWLLCCPVILIHLSNVTGLSDDYGRRTMGLLVSDIATIVFGVTAAMLVNWPKIIFYLIGFTMCCYTFFLAAKVLIESFHQVPKGICRHLVKAMAITYFVGWSFFPLIFLFGQSGFKKISPYADVIASSFGDLISKNAFGMLGHFLRVKIHEHILKHGDIRKTTHLRIAGEEKEVETFVEEEDEDTAKHSTKELANRGSFIVMRDKMKEQGIDVRASLDMDEDEEARTGKGKGAGATSLVPGRVILAVPDISMVDFFHDHFAHLGASIELVPALGVENTLLLVQQAMQLGGLDFVLVHPEFLRDRSQNGLVSRLKMTGHGVCAFGWVPSGPMREIIESAGVDGWLDGPSFGTGIDQEQLIELIGYMQAKRKFGMRFGGGGASKAGYSSDGGFGGKGMLEMQPSMSQGSGVPLLQQNNSMMRAPPSPMGNMANNGMMNPMMSMNNPMMGGGAVMMTSMGSMQQAANPLYGAPPSPLSSQPGAGMYGAPAQPQMGSQGSMHGSMYGGSQQQHQQPQQAAAAPAAADGGSEAEMLKQLMSEINRLKAELGES

SEQ ID NO: 103 - protein, 310 aa, synthetic construct
  REGION 1..310: note = synthetic construct of Channel rhodopsin 2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 104 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 105 - protein, 872 aa, Homo sapiens [sequence omitted]

SEQ ID NO: 106 - protein, 2221 aa, Homo sapiens
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIALKILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKGYFSDPWNVFDFLIVIGSIIDVILSETNHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRERHVPMCEDLELRRDSGSAGTQAHCLLLRKANPSRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGRPSEEELQDSRVYVSSL